(19) United States
(12) Patent Application Publication
(10) Pub. No.: US 2024/0132589 A1
(43) Pub. Date: Apr. 25, 2024

(54) LILRB2 ANTIBODY PRODUCTS AND METHODS

(71) Applicant: Oncoresponse, Inc., Seattle, WA (US)

(72) Inventors: Meghan Zuck, Seattle, WA (US); Peter Probst, Seattle, WA (US); Thomas J. Graddis, Seattle, WA (US); Myriam N. Bouchlaka Bay, Seattle, WA (US); Kamal D. Puri, Seattle, WA (US)

(21) Appl. No.: 18/488,833

(22) Filed: Oct. 17, 2023

Related U.S. Application Data
(60) Provisional application No. 63/514,057, filed on Jul. 17, 2023.
(60) Provisional application No. 63/490,705, filed on Mar. 16, 2023.
(60) Provisional application No. 63/380,034, filed on Oct. 18, 2022.

(51) Int. Cl. C07K 16/28; A61P 35/00; C12N 15/63

(52) U.S. Cl. CPC C07K 16/2803; A61P 35/00; C12N 15/63; C07K 2317/14; C07K 2317/51; C07K 2317/565; C07K 2317/92

(57) ABSTRACT

The disclosure relates to LILRB2 antibody products and methods of use thereof. The antibody products specifically bind to LILRB2 on cells such as myeloid cells or cancer cells. The antibody products can be used in methods of treatment of disease, such as methods cancer immunotherapy.

### CROSS-REFERENCE TO RELATED APPLICATIONS

This application claims priority to Provisional Application No. 63/380,034, filed Oct. 18, 2022, U.S. Provisional Application No. 63/490,705, file Mar. 16, 2023 and U.S. Provisional Application No. 63/514,057, filed Jul. 17, 2023, all of which are incorporated herein by reference in their entirety.

### INCORPORATION BY REFERENCE OF THE SEQUENCE LISTING

This application contains, as a separate part of disclosure, a Sequence Listing in computer-readable form (Filename: 58365_Seqlisting.XML; 100,193 bytes dated Oct. 13, 2023) which is incorporated by reference herein in its entirety.

### FIELD

The disclosure relates to LILRB2 antibody products and related methods. The products disclosed herein bind to LILRB2 on cells such as macrophages or cancer cells. The products can be used in methods of treatment of disease, such as in methods of treating cancer.

### BACKGROUND

Cancer is characterized by the accumulation of growth-modifying genetic alterations, along with the ability to evade detection or elimination by the immune system. Cancer immunotherapy allows one to overcome this immune tolerance, leading to immune recognition and anti-tumor responses. However, tumors typically use multiple mechanisms to mediate immune evasion, such as reduction or loss of tumor antigenicity, immune evasion, or the presence of suppressive cellular or molecular factors in the tumor microenvironment (TME) that inhibit the killing of cancer cells by CD8+ T cells.

The recent development of antibodies that target immune checkpoints has transformed the way malignant solid tumors are treated and has provided hope that even subjects with advanced disease may achieve a cure or long-term remission. Checkpoint inhibitor (CPI) antibodies targeting cytotoxic T-lymphocyte-associated protein 4 (CTLA-4), programmed death-1 (PD-1) and programmed death ligand-1 (PD-L1) have now established clear efficacy for first-line treatment of metastatic disease in numerous tumor types, including lung, melanoma, triple-negative breast cancer, head and neck and others.

Despite this success, most subjects treated with these CPIs will either not respond or will ultimately progress or relapse. In addition, CPIs have failed to improve the outcomes of those with tumors that are not infiltrated by immune cells, so-called ‘cold’ tumors. This lack of responsiveness and treatment failure in cold tumors is thought to be, in part, due to an immune suppressive TME. Immunosuppressive myeloid cells such as myeloid derived suppressor cells (MDSC) and tumor associated macrophages (TAM) are important components of the TME and contribute to immune evasion by many solid tumors. MDSCs and TAMs suppress anti-tumor immune responses and promote a tumorigenic milieu. High levels of tumor infiltration by MDSCs and TAMs generally predict an unfavorable prognosis in solid tumor subjects. Relieving the immune suppression of myeloid cells in the TME to improve T-cell-mediated responses is a rational adjunct to CPI therapy.

Leukocyte immunoglobulin-like receptor B2 (LILRB2, also known as ILT4, LIR2, MIR-10, CD85d) is an ITIM containing immunosuppressive member of the leukocyte immunoglobulin-like receptor family that is expressed on cells of the myeloid lineage (monocytes, macrophages, dendritic cells, and granulocytes) but not on lymphocytes. In the TME, LILRB2 is found on myeloid-derived suppressor cells (MDSCs) and tumor-supporting tumor-associated macrophages (TAMs). LILRB2 appears to have a dual role in cancer biology, as an immune checkpoint on myeloid cells and as a tumor-supporting factor when expressed on tumor cells. Trans or cis interactions of LILRB2 with its ligands mediate immune suppression by myeloid cells and promote tumor immune evasion in the TME. Such ligands include human leukocyte antigen G (HLA-G), a nonclassical MHC class I molecule, as well as the classical HLA class I molecules HLA-A and HLA-B. Targeting this pathway in the TME may enhance efficacy of T cell checkpoint inhibitors. In addition, ANGPTL2 and ANGPTL5 promote lung cancer development and survival via tumor-expressed LILRB2 by SHP1 signaling LILRB2 expression on myeloid cells in the TME or HLA-G expression by tumors correlates with poor survival in multiple cancers.

Recent data indicate that LILRB2 blockade in combination with a cytokine (e.g., IL-4, IL-10) or an innate signal of differentiation/polarization promotes the development of anti-tumoral myeloid cells in vitro. In addition, combination of an anti-LILRB2 antibody with anti PD-1 treatment enhanced the tumor growth inhibition (TGI) in a humanized tumor model.

Antibodies that target and antagonize LILRB2 are currently being evaluated in clinical trials for the treatment of cancer, for example, MK-4830 (IgG4) (Agenus and Merck) and JTX-8064 (IgG4) (Jounce). Initial clinical data with the first in class anti-LILRB2 MK-4830 suggest that LILRB2 blockade abrogates a PD-1 resistance mechanism in patients with advanced solid tumors. MK-4830 dosed as monotherapy or in combination with pembrolizumab (anti-PD-1) was well tolerated and demonstrated dose-related evidence of target engagement and anti-tumor activity in patients that lacked predictive biomarkers associated with response to anti-PD-1 monotherapy. The overall response rate of MK-4830/pembrolizumab combination treatment was 24%. The MK-4830 clinical trial data supports the development of anti-LILRB2 antibodies in combination with CPI therapy.

PCT Publication No. WO 2021/138079 A1 discloses use of a combination of a PD-1 antagonist, an ILT4 (LILRB2) antagonist, and lenvatinib (a kinase inhibitor) for the treatment of cancers.

There remains a need in the art for LILRB2 antibody products and methods for their therapeutic use.

### SUMMARY

The disclosure provides LILRB2 antibody products (also sometimes referred to herein as “anti-LILRB2 antibody products” herein), compositions comprising such antibody products, and methods of their manufacture and use in the treatment of disease, including cancer.

An LILRB2 antibody product provided herein can be an intact antibody or can comprise an immunologically functional antibody fragment and thus LILRB2 antibody products include antibodies with a naturally-occurring structure or recombinant structure, as well as other polypeptides, such as antibody fragments, that have an antigen-binding domain. Nucleic acids molecules, vectors, and host cells useful in the production of the antibody products are also provided. The antibody products and compositions thereof can be used to prevent or treat a variety of different disease conditions including, but not limited to, preventing or treating disease conditions such as cancer. LILRB2 antibodies provided herein have a useful half-life and display anti-tumor activity in humanized-mouse models. Humanized LILRB2 antibodies provided herein restore effector function of activated and exhausted T cells from M2c-mediated immune suppression and enhance secretion of proinflammatory cytokines by M0 macrophages or LPS stimulated PBMCs.

It is believed that the LILRB2 antibody products provided herein bind LILRB2 at an epitope that is distinct from those bound by antibodies currently in the clinic. In preclinical studies, the antibody products enhanced LPS-induced IFN-γ production and reduced IL-10 release by peripheral blood mononuclear cells (PBMCs) and relieve immune suppression of pro-tumoral macrophages to induce proliferation of, and IFN-γ and perforin secretion by, T cells. In addition, the antibody products inhibit the development of immunosuppressive macrophages. In contrast to a comparator LILRB2 antibody, a representative antibody product has demonstrated the ability to restore the ability of exhausted T cells to secrete IFN-γ in the presence of pro-tumoral macrophages and has also significantly enhanced the activity of pembrolizumab in combination studies. Importantly, a chimeric antibody product demonstrated superior anti-tumor activity over the comparator with significant tumor growth inhibition and tumor regression in humanized mouse tumor model.

Without wishing to be bound by any particular theory, it contemplated herein that the anti-immunosuppressive activity of the antibodies provided appears to arise from two distinct but coordinated mechanisms: variable region engagement of LILRB2 and heavy chain engagement of Fc receptors.

On the one hand, it is believed that LILRB2 expressed on myeloid cells negatively regulates anti-tumor immunity via binding to HLA-G on tumor cells and MHC Class 1 on the myeloid cells. Binding of LILRB2 to these ligands promotes immunosuppressive myeloid cells in the tumor microenvironment via inhibition of Ca²⁺ signaling across the cell membranes of the myeloid cells, recruitment of Src-homology domain 2 containing protein tyrosine phosphatase-1 (SHP1) and SHP2 phosphatases within the myeloid cell membranes, and production by the myeloid cells of cytokines that can prevent checkpoint inhibitor (CPI) therapies from potentiating anti-tumor T cell responses. Individually or collectively, these phenomena can result in reduced killing of cancer cells. It is also believed that LILRB2 further impedes tumor cell killing by competing with cytotoxic T lymphocytes for binding to MHC class I.

On the other hand, activating Fc receptors (FcγRIA, FcγRIIA, and FcγRIIIA) on myeloid cells is known to play an essential role in promoting cell activation, differentiation, and induction of adaptive immune responses through the modulation of antigen presentation. These receptors signal through an immunoreceptor tyrosine-based activation motif via SRC family kinases and spleen tyrosine kinase leading to transcriptional activation of several pro-inflammatory cytokines and chemokines driving cell mobilization, migration, differentiation, and survival.

The antibodies disclosed herein appear to enable coupling of LILRB2 antagonism with Fc receptor-mediated activation. The antibodies bind a unique epitope on LILRB2 to not only block myeloid cell interactions with tumor cell HLA-G, but also cis-interactions with MHC class I, thereby deactivating both inhibitory signals in the myeloid cells. This action can promote the polarization of tumor-infiltrating myeloid cells toward an inflammatory phenotype, while simultaneously freeing MHC Class I on these cells to engage receptors required for optimal activation of cytotoxic T lymphocytes. Moreover, the antibodies can enhance anti-tumor immunity by providing an immunostimulatory signal through engagement of activating FcγRIIIA. This dual, or co-engagement, mechanism provides these antibodies with novel attributes for targeting myeloid cells to reverse CPI resistance, enhance tumor cell killing, and improve patient outcomes.

Non-clinical data for a representative antibody product demonstrates a typical human IgG PK profile in humanized FcRn mice. The antibody products do not cross-react with non-human primate LILRB2 and show no cross-reactivity to other inhibitory or activating LILR family members. Treatment with representative antibody products did not trigger inflammatory cytokine release or activation of neutrophils in human whole blood and representative antibody products did not deplete human monocytes or neutrophils suggesting a tolerable safety profile.

The disclosure provides an antibody product that binds human LILRB2, the antibody product comprising a CDR-H1 set forth in SEQ ID NO: 16, a CDR-H2 set forth in SEQ ID NO: 17, a CDR-H3 set forth in SEQ ID NO: 24, a CDR-L1 set forth in SEQ ID NO: 19, a CDR-L2 set forth in SEQ ID NO: 20, and a CDR-L3 set forth in SEQ ID NO: 21. The antibody product can comprise a CDR-H1 set forth in SEQ ID NO: 16, a CDR-H2 set forth in SEQ NO: 17, a CDR-H3 set forth in SEQ ID NO: 18, a CDR-L1 set forth in SEQ ID NO: 19, a CDR-L2 set forth in SEQ ID NO: 20, and a CDR-L3 set forth in SEQ ID NO: 21. The antibody product can comprise a CDR-H1 set forth in SEQ ID NO: 22, a CDR-H2 set forth in SEQ NO: 23, a CDR-H3 set forth in SEQ ID NO: 24, a CDR-L1 set forth in SEQ ID NO: 25, a CDR-L2 set forth in SEQ ID NO: 26, and a CDR-L3 set forth in SEQ ID NO: 21. The antibody product can comprise a heavy chain variable region comprising: (a) an amino acid sequence at least 80% identical to SEQ ID NO: 1, 6, 7, 8, 9 or 10; or (b) an amino acid sequence set forth in SEQ ID NO: 1, 6, 7, 8, 9 or 10. The antibody product can comprise a light chain variable region comprising: (a) an amino acid sequence at least 80% identical to SEQ ID NO: 2, 11, 12, 13, 14 or 15; or (b) an amino acid sequence set forth in SEQ ID NO: 2, 11, 12, 13, 14 or 15. The antibody product can comprise: (a) a heavy chain variable region comprising SEQ ID NO: 1; and a light chain variable region SEQ ID NO: 2; (b) a heavy chain variable region SEQ ID NO: 6; and a light chain variable region comprising SEQ ID NO: 11; (c) a heavy chain variable region comprising SEQ ID NO: 6; and a light chain variable region comprising SEQ ID NO: 12; (d) a heavy chain variable region comprising SEQ ID NO: 6; and a light chain variable region comprising SEQ ID NO: 13; (e) a heavy chain variable region comprising SEQ ID NO: 6; and a light chain variable region comprising SEQ ID NO: 14; (f) a heavy chain variable region comprising SEQ ID NO: 6; and a light chain variable region comprising SEQ ID NO: 15; (g) a heavy chain variable region comprising SEQ ID NO: 7; and a light chain variable comprising SEQ ID NO: 11; (h) a heavy chain variable region comprising SEQ ID NO: 7; and a light chain variable region comprising SEQ ID NO: 12; (i) a heavy chain variable region comprising SEQ ID NO: 7; and a light chain variable region comprising SEQ ID NO: 13; (j) a heavy chain variable region comprising SEQ ID NO: 7; a light chain variable region comprising SEQ ID NO: 14; (k) a heavy chain variable region comprising SEQ ID NO: 7; and a light chain variable region comprising SEQ ID NO: 15; (l) a heavy chain variable region comprising SEQ ID NO: 8; and a light chain variable region comprising SEQ ID NO: 11; (m) a heavy chain variable region comprising SEQ ID NO: 8; and a light chain variable region comprising SEQ ID NO: 12; (n) a heavy chain variable region comprising SEQ ID NO: 8; and a light chain variable region comprising SEQ ID NO: 13; (o) a heavy chain variable region comprising SEQ ID NO: 8; and a light chain variable region comprising SEQ ID NO: 14; (p) a heavy chain variable region comprising SEQ ID NO: 8; and a light chain variable region comprising SEQ ID NO: 15; (q) a heavy chain variable region comprising SEQ ID NO: 9; a light chain variable region comprising SEQ ID NO: 11; (r) a heavy chain variable region comprising SEQ ID NO: 9; and a light chain variable region comprising SEQ ID NO: 12; (s) a heavy chain variable region comprising SEQ ID NO: 9; and a light chain variable region comprising SEQ ID NO: 13; (t) a heavy chain variable region comprising SEQ ID NO: 9; and a light chain variable region comprising SEQ ID NO: 14; (u) a heavy chain variable region comprising SEQ ID NO: 9; and a light chain variable region comprising SEQ ID NO: 15; (v) a heavy chain variable region comprising SEQ ID NO: 10; and a light chain variable region comprising SEQ ID NO: 11; (w) a heavy chain variable region comprising SEQ ID NO: 10; and a light chain variable region comprising SEQ ID NO: 12; (x) a heavy chain variable region comprising SEQ ID NO: 10; and a light chain variable region comprising SEQ ID NO: 13; (y) a heavy chain variable region comprising SEQ ID NO: 10; and a light chain variable region comprising SEQ ID NO: 14; or (z) a heavy chain variable region comprising SEQ ID NO: 10; and a light chain variable region comprising SEQ ID NO: 15.

The antibody product can comprise a heavy chain comprising the heavy chain variable domain (VH) and a human heavy chain constant domain (CH). An antibody product can comprise a light chain comprising the light chain variable domain (VL) and a human light chain constant domain (CL). An antibody product can comprise a heavy chain comprising the heavy chain variable domain (VH) and a human heavy chain constant domain (CH) and a light chain comprising the light chain variable domain (VL) and a human light chain constant domain (CL). The antibody product can comprise a heavy chain region comprising: (a) an amino acid sequence at least 80% identical to SEQ ID NO: 32; or (b) an amino acid sequence set forth in SEQ ID NO: 32. The antibody product can comprise a light chain region comprising an amino acid sequence at least 80% identical to SEQ ID NO: 42; or (b) an amino acid sequence set forth in SEQ ID NO: 42. An illustrative antibody product comprises a heavy chain comprising an amino acid sequence set forth in SEQ ID NO: 32 and a light chain region comprising an amino acid sequence set forth in SEQ ID NO: 42.

The antibody product can comprise an IgA, IgD, IgE, IgG, or IgM heavy chain constant domain. The antibody product can comprise an IgG1 constant domain, an IgG2 constant domain, or an IgG4 constant domain. The antibody product can comprise an IgG1 constant domain. The antibody product can comprise an IgG1 heavy chain amino acid sequence comprising: (a) an amino acid sequence at least 80% identical to SEQ ID NO: 3, SEQ ID NO: 28, SEQ ID NO: 29, SEQ ID NO: 30, SEQ ID NO: 31, or SEQ ID NO: 32; or (b) an amino acid sequence set forth in to SEQ ID NO: 3, SEQ ID NO: 28, SEQ ID NO: 29, SEQ ID NO: 30, SEQ ID NO: 31, or SEQ ID NO: 32. The antibody product can comprise an IgG4 constant domain. The antibody product can comprise an IgG4 heavy chain amino acid sequence comprising: (a) an amino acid sequence at least 80% identical to SEQ ID NO: 4, SEQ ID NO: 33, SEQ ID NO: 34, SEQ ID NO: 35, SEQ ID NO: 36, or SEQ ID NO: 37; or (b) an amino acid sequence set forth in to SEQ ID NO: 4, SEQ ID NO: 33, SEQ ID NO: 34, SEQ ID NO: 35, SEQ ID NO: 36, or 37.

The antibody product can comprise: (a) an IgG1 heavy chain having an amino acid sequence comprising SEQ ID NO: 1; and a kappa light chain having an amino acid sequence comprising SEQ ID NO: 2; (b) an IgG1 heavy chain having an amino acid sequence comprising SEQ ID NO: 6; and a kappa light chain having an amino acid sequence comprising SEQ ID NO: 11; (c) an IgG1 heavy chain having an amino acid sequence comprising SEQ ID NO: 6; and a kappa light chain having an amino acid sequence comprising SEQ ID NO: 12; (d) an IgG1 heavy chain having an amino acid sequence comprising SEQ ID NO: 6; and a kappa light chain having an amino acid sequence comprising SEQ ID NO: 13; (e) an IgG1 heavy chain having an amino acid sequence comprising SEQ ID NO: 6; and a kappa light chain having an amino acid sequence comprising SEQ ID NO: 14; (f) an IgG1 heavy chain having an amino acid sequence comprising SEQ ID NO: 6; and a kappa light chain having an amino acid sequence comprising SEQ ID NO: 15; (g) an IgG1 heavy chain having an amino acid sequence comprising SEQ ID NO: 7; and a kappa light chain having an amino acid sequence comprising SEQ ID NO: 11; (h) an IgG1 heavy chain having an amino acid sequence comprising SEQ ID NO: 7; and a kappa light chain having an amino acid sequence comprising SEQ ID NO: 12; (i) an IgG1 heavy chain having an amino acid sequence comprising SEQ ID NO: 7; and a kappa light chain having an amino acid sequence comprising SEQ ID NO: 13; (j) an IgG1 heavy chain having an amino acid sequence comprising SEQ ID NO: 7; and a kappa light chain having an amino acid sequence comprising SEQ ID NO: 14; (k) an IgG1 heavy chain having an amino acid sequence comprising SEQ ID NO: 7; and a kappa light chain having an amino acid sequence comprising SEQ ID NO: 15; (l) an IgG1 heavy chain having an amino acid sequence comprising SEQ ID NO: 8; and a kappa light chain having an amino acid sequence comprising SEQ ID NO: 11; (m) an IgG1 heavy chain having an amino acid sequence comprising SEQ ID NO: 8; and a kappa light chain having an amino acid sequence comprising SEQ ID NO: 12; (n) an IgG1 heavy chain having an amino acid sequence comprising SEQ ID NO: 8; and a kappa light chain having an amino acid sequence comprising SEQ ID NO: 13; (o) an IgG1 heavy chain having an amino acid sequence comprising SEQ ID NO: 8; and a kappa light chain having an amino acid sequence comprising SEQ ID NO: 14; (p) an IgG1 heavy chain having an amino acid sequence comprising SEQ ID NO: 8; and a kappa light chain having an amino acid sequence comprising SEQ ID NO: 15; (q) an IgG1 heavy chain having an amino acid sequence comprising SEQ ID NO: 9; and a kappa light chain having an amino acid sequence comprising SEQ ID NO: 11; (r) an IgG1 heavy chain having an amino acid sequence comprising SEQ ID NO: 9; and a kappa light chain having an amino acid sequence comprising SEQ ID NO: 12; (s) an IgG1 heavy chain having an amino acid sequence comprising SEQ ID NO: 9; and a kappa light chain having an amino acid sequence comprising SEQ ID NO: 13; (t) an IgG1 heavy chain having an amino acid sequence comprising SEQ ID NO: 9; and a kappa light chain having an amino acid sequence comprising SEQ ID NO: 14; (u) an IgG1 heavy chain having an amino acid sequence comprising SEQ ID NO: 9; and a kappa light chain having an amino acid sequence comprising SEQ ID NO: 15; (v) an IgG1 heavy chain having an amino acid sequence comprising SEQ ID NO: 10; and a kappa light chain having an amino acid sequence comprising SEQ ID NO: 11; (w) an IgG1 heavy chain having an amino acid sequence comprising SEQ ID NO: 10; and a kappa light chain having an amino acid sequence comprising SEQ ID NO: 12; (x) an IgG1 heavy chain having an amino acid sequence comprising SEQ ID NO: 10; and a kappa light chain having an amino acid sequence comprising SEQ ID NO: 13; (y) an IgG1 heavy chain having an amino acid sequence comprising SEQ ID NO: 10; and a kappa light chain having an amino acid sequence comprising SEQ ID NO: 14; or (z) an IgG1 heavy chain having an amino acid sequence comprising SEQ ID NO: 10; and a kappa light chain having an amino acid sequence comprising SEQ ID NO: 15.

The antibody product can specifically bind to human LILRB2, for example, with a KD from 0.5 nM to 500 nM.

The antibody product can specifically bind human LILRB2 expressed by myeloid cells or cells, such as LILRB2-expressing cells, of a cancer. The antibody product can specifically bind to a human immunosuppressive myeloid cell. The immunosuppressive myeloid cell can be in a tumor microenvironment. The immunosuppressive myeloid cell can be a macrophage, a myeloid dendritic cell, or a myeloid-derived suppressor cell. The immunosuppressive myeloid cell can be a M2a, M2b, M2c, or M2d macrophage. The antibody product can specifically bind to human M2c macrophages with a KD, for example, from 0.5 nM to 500 nM.

The antibody product can be a monoclonal antibody, a human antibody, a chimeric antibody, a humanized antibody, or a single chain antibody. The antibody product can be a monospecific, bispecific, trispecific, or multispecific antibody.

The antibody product can be bound by an Fc receptor expressed on an immunosuppressive macrophage or other myeloid cell. The antibody product can be bound by CD16 (FcγRIII) (e.g., FcγRIIIa, FcγRIIIb), CD32 (FcγRII), or CD64 (FcγRI) expressed on an immunosuppressive macrophage or other myeloid cell.

The disclosure provides methods of cancer immunotherapy to a subject in need thereof, in which the cancer is associated with a presence of immunosuppressive macrophages, the method comprising administering to the subject a therapeutically effective amount of an antibody product provided herein. In the methods, the antibody product binds to a macrophage and binding of the antibody product to a macrophage can results in at least one of the following effects: (a) promotes activation of a CD4+ T cell, CD8+ T cell, NK cell, or any combination thereof; (b) promotes proliferation of a CD4+ T cell, CD8+ T cell, NK cell, or any combination thereof; (c) prevents the polarization of macrophages to immunosuppressive phenotype; and (d) enhances the innate anti-tumor response. The activation of a CD4+ T cell, CD8+ T cell, NK cell, or any combination thereof can be measured as an increased level of IFN-γ, TNF-α, or perforin, or any combination thereof. In the methods, binding of the antibody product to a macrophage may or may not be cytotoxic to the macrophage. In the methods, binding of the antibody product to a macrophage can result in at least one of the following effects: (a) internalization of the antibody product by the macrophage; (b) secretion of TNFα, IL-6, perforin, or any combination thereof; (c) reduced release of IL-10; (d) activation of a CD4+ T cell, CD8+ T cell, NK cell, or any combination thereof; (e) proliferation of a CD4+ T cell, CD8+ T cell, NK cell, or any combination thereof; and (f) promotion of tumor cell killing in a tumor microenvironment. The methods can result in: two or more of (a) through (f); three or more of (a) through (f); four or more of (a) through (f); five of more of (a) through (f) or all of (a) through (f). In the methods, binding of the antibody product to a macrophage can increase an immunostimulatory activity in a tumor microenvironment. Binding of the antibody product to a macrophage can reduce an immunosuppressive activity of the macrophage. Binding of the antibody product to a macrophage can reduce a tumor-promoting activity of the macrophage. Binding of the antibody product can promote CD4+ T cell activation, CD4+ T cell proliferation, or both CD4+ T cell activation and proliferation. Binding to the antibody product can promote CD8+ T cell activation, CD8+ T cell proliferation, or both CD8+ T cell activation and proliferation. Binding of the antibody product can promote cytotoxic lymphocyte-mediated killing of cancer cells. Binding of the antibody product can promote NK cell-mediated tumor cell killing. Binding of the antibody product to a macrophage can reduce suppression of cytotoxic T cell-mediated killing of tumor cells in the tumor microenvironment. The cancer can be, for example, a sarcoma, carcinoma, or blood-borne cancer. The cancer can be, for example, a glioblastoma multiforme, head and neck cancer, kidney renal clear cell cancer, acute myeloid leukemia, pancreatic adenocarcinoma, skin cutaneous melanoma, stomach adenocarcinoma, testicular germ cell cancer, gastric cancer, Merkel cell carcinoma, dendritic sarcoma, non-small cell lung cancer, papillary thyroid cancer, cutaneous squamous cell carcinoma, or ovarian cancer. The cells of the cancer can express or overexpress LILRB2.

The methods provided herein can further comprise administering to the subject an effective amount of an anti-cancer therapeutic. The anti-cancer therapeutic can be an immune checkpoint inhibitor including, but not limited to, a PD-1 antagonist. The effective amount of the PD-1 antagonist can be an amount effective to relieve immunosuppression of T cells. The immunosuppression of T cells can be mediated through interaction of the T cells with myeloid cells expressing PD-L1.

The disclosure provides compositions comprising (a) the antibody product provided herein and (b) an excipient. The disclosure provides an article of manufacture, comprising a composition provided herein and a container.

The disclosure contemplates the use of an antibody product or composition provided herein, for manufacture of a medicament for treatment of cancer in a subject in need thereof, wherein the cancer expresses LILRB2.

The disclosure provides an isolated nucleic acid, comprising a nucleotide sequence encoding part or all of an antibody product provided herein. The disclosure provides expression vectors comprising nucleic acids provided herein. The disclosure provides a host cell comprising an expression vector provided herein.

The disclosure provides a method of producing an immunoglobulin heavy chain variable domain or an immunoglobulin light chain variable domain, the method comprising: (a) growing the host cell provided herein under conditions such that the host cell expresses the protein comprising the immunoglobulin heavy chain variable region or the immunoglobulin light chain variable region; and (b) purifying the protein comprising the immunoglobulin heavy chain variable domain or the immunoglobulin light chain variable domain.

The disclosure provides a pharmaceutical composition comprising an antibody product provided herein, and a pharmaceutically acceptable excipient. The pharmaceutical composition can be used for treating a subject having a cancer expressing LILRB2.

The disclosure provides a method of detecting LILRB2 in a sample, tissue, or cell using the antibody product provided herein, comprising contacting the sample, tissue or cell with the antibody product and detecting the antibody product.

The disclosure provides a method of reducing a biological activity of LILRB2 in a subject in need thereof, said method comprising administering a therapeutically effective amount of an antibody product or a pharmaceutical composition provided herein to the subject. In the method, the antibody product can mediate depletion of at least one cancer cell expressing LILRB2.

The disclosure provides a method of promoting an immune response in a subject in need thereof, the method comprising administering a therapeutically effective amount of the antibody product or the pharmaceutical composition provided herein.

The disclosure provides a method of providing a cancer immunotherapy to a subject in need thereof, in which cancer cells of the subject express LILRB2, the method comprising administering to the subject a therapeutically effective amount of an antibody product provided herein. The method can comprise administering an amount the antibody product effective to mediate killing of cells of the cancer through antibody-dependent cellular toxicity. The method can comprise administering an amount of the antibody product effective to relieve LILRB2-mediated suppression of T cells in the subject. The method can further comprise administering to the subject a PD-1 antagonist in an amount sufficient to relieve PD-1/PD-L1 axis-mediated immunosuppression of T cells in the subject. The PD-1 antagonist can be a PD-1 antibody product.

An antibody product provided herein can be detectably labeled or can comprise a conjugated toxin, drug, receptor, enzyme, receptor ligand. The antibody product can comprise a therapeutic or cytotoxic agent.

A pharmaceutical composition comprising an antibody product provided herein, can reduce or prevent binding of LILRB2 to a ligand thereof and/or reduces or prevents LILRB2-mediated signal transduction and a physiologically acceptable carrier or excipient. The ligand can be human leukocyte antigen A, human leukocyte antigen B, human leukocyte antigen C, human leukocyte antigen G, angiopoietin-like protein 2, angiopoietin-like protein 5, or a combination thereof. The ligand is expressed on the surface of a myeloid cell or a tumor cell.

The disclosure provides methods of treating cancer in a subject in need thereof, comprising administering to the subject an effective amount of a pharmaceutical composition provided herein, wherein the subject has cancer comprising cells expressing or over-expressing a ligand of LILRB2. In the methods, the antibody product or antigen binding fragment thereof increases an immune response, retards or prevents tumor growth, inhibits tumor-mediated immune suppression, eliminate tumors, depletes or blocks the activity of tumor-associated macrophages so as to alter their activity, decreases tumor-associated macrophage-mediated immune suppression, reduces or reverses T cell suppression, or any combination thereof. The cancer or tumor can comprise macrophages expressing LILRB2. The methods can further comprise administering to the subject a second therapeutic agent. The second therapeutic agent can be an immune checkpoint inhibitor.

The following Drawings and Detailed Description (including the Examples) illustrate various non-limiting aspects of the subject matter contemplated herein.

### BRIEF DESCRIPTION OF THE DRAWINGS

Aspects of the disclosure are illustrated by the following figures.

FIG. 1A-B: Binding of B2A-IgG1 and B2A-IgG4 chimera to human and cynomolgus LILRB2 Fc by ELISA and on LILRB2 expressing HEK293 cells.

FIG. 2: B2A-IgG4 blocking of LILRB2 binding to human ANGPTL-2, -5 by ELISA.

FIG. 3A-C: Human anti-LILRB2 chimeric antibodies binding curves to human monocytes and M0 and M2c macrophages.

FIG. 4: Enhanced IFN-γ secretion by PBMC stimulated with LPS following treatment with anti-LILRB2 chimeric B2A-IgG1 and B2A-IgG4.

FIG. 5: Enhanced TNF-α secretion by human M0 macrophages following treatment with chimeric B2A-IgG1 and B2A-IgG4 antibodies in CD40 ligand assay.

FIG. 6A-B: Rescue of CD8+ T cell proliferation with chimeric LILRB2 antibodies in M2c and CD8+ T cell coculture assay, and rescue of IFN-γ and Perforin responses with chimeric LILRB2 antibodies in M2c and CD8+ T cell coculture assay.

FIG. 7: Chimeric B2A-IgG1 LILRB2 antibody rescues IFN-γ release by exhausted T cells from M2c-mediated immune suppression.

FIG. 8: Pharmacokinetic profiling of chimera B2A-IgG4 and B2A-IgG1 in humanized FcRn mice.

FIG. 9A-B: Anti-tumor efficacy of chimeric B2A-IgG4 in humanized NSG-SGM3 mice and subcutaneous SK-MEL-5 human melanoma tumor model.

FIG. 10: Humanized LILRB2 variants binding curves to monocytes, M0 and M2c macrophages.

FIG. 11: Humanized LILRB2 antibodies bind a variety of human myeloid cells, including classical, intermediate, and non-classical monocytes, and myeloid dendritic cells.

FIG. 12: Humanized LILRB2 variants bind to human neutrophils.

FIG. 13: Humanized anti-LILRB2 variant B2H1-55 does not elicit neutrophil activation in whole blood of healthy subjects.

FIG. 14: Humanized LILRB2 antibodies elicit IFN-γ secretion by LPS-stimulated PBMC.

FIG. 15: Rescue of CD8+ T cells proliferation and cytokine responses with humanized LILRB2 antibodies in M2c and CD8+ T cell proliferation assay.

FIG. 16: Humanized LILRB2 variant prevent development of immunosuppressive macrophages.

FIG. 17A-B: Relief of M2c-mediated immune suppression in M2c/CD4+ T Cell coculture with select humanized variants B2H1-55.

FIG. 18: Humanized LILRB2 antibodies rescue IFN-γ release by exhausted T cells from M2c-mediated immune suppression.

FIG. 19: Humanized anti-LILRB2 variant B2H1-55 enhances IFN-γ release by exhausted T cells from M2c-mediated immune suppression in combination with PD-1 antibody.

FIG. 20: Humanized anti-LILRB2 variants elicit minimal cytokine response in whole blood of healthy subjects.

FIG. 21A-B: Humanized anti-LILRB2 variants do not mediate ADCC of human monocytes and induce ADCC on HEK293 expressing human LILRB2.

FIG. 22: Pharmacokinetic profile of IgG1 humanized anti-LILRB2 variants in humanized FcRn mice.

FIG. 23: Humanized anti-LILRB2 variants inhibit LPS-mediated IL-10 secretion by human PBMC.

### DETAILED DESCRIPTION

Unless otherwise defined herein, scientific and technical terms used herein have the meanings that are commonly understood by those of ordinary skill in the art to which the claimed subject matter belongs.

### Antibody Products

The disclosure provides antibody products that specifically bind to human LILRB2.

“PD-1 antagonist” means any chemical compound or biological molecule that blocks binding of PD-1 expressed on an immune cell (T cell, B cell or NKT cell) to PD-L1 expressed on a cancer cell, and preferably also blocks binding of the immune-cell expressed PD-1 to PD-L2 expressed on a cancer cell. Alternative names or synonyms are given for PD-1 and its ligands. For PD-1: PDCD1, PD1, CD279 and SLEB2; for PD-L1: PDCD1L1, PDL1, B7H1, B7-4, CD274 and B7-H; and for PD-L2: PDCD1L2, PDL2, B7-DC, Btdc, and CD273. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP 054862 and NP 079515, respectively.

“LILRB2 antagonist” means any chemical compound or biological molecule that blocks binding of LILRB2 to HLA-G, HLA-A, HLA-B, HLA-F, or an angiopoietin-like protein (ANGPTL, such as ANGPTL2, or ANGPTL5,). Alternative names or synonyms for LILRB2 and its ligands include but are not limited to: ILT4, ILT-4, MIR10, MIR-10, LIR2, LIR-2, CD85D for ILT4; MHC-G or major histocompatibility complex, class I, G for HLA-G; major histocompatibility complex, class I, A for HLA-A; AS, B-4901, major histocompatibility complex, class I, B for HLA-B; CDA12, HLA-CDA12, or major histocompatibility complex, class I, F for HLA-F; angiopoietin-3, ANG3, ANGPT3, ARP1, UNQ162, angiopoietin like 2 for ANGPTL2; ARP4, HF ARP, PGAR, UNQ171, angiopoietin like 54 for ANGPTL5; and CDT6. In any of the treatment methods, medicaments and disclosed uses in which a human individual is being treated, the LILRB2 antagonist blocks binding of human LILRB2 to human HLA-G, HLA-A, HLA-B, HLA-C, HLA-F, ANGPTL2, or ANGPTL5. Human LILRB2 precursor amino acid sequence can be found in NCBI Locus No.: AAB88119.1. Human HLA-G, HLA-A, HLA-B, HLA-C and HLA-F precursor amino acid sequences can be found in NCBI Locus No.: P17693.1, P04439.2, P01889.3, P10321-1, and P30511.3, respectively. Human ANGPTL2, and ANGPTL5 precursor amino acid sequences can be found in NCBI Locus No. Q9UKU9-1 and Q86XS5, respectively.

The protein sequence of human LILRB2 (NM_005874.5) is set out in SEQ ID NO: 47. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. Multiple transcript variants encoding different isoforms have been found for this gene. The nucleotide sequence of a human cDNA encoding LILRB2 (NM_005874.5) is set out in SEQ ID NO: 53.

The terms “polypeptide” and “protein” are used interchangeably herein in the conventional way to refer to a molecule formed of amino acids. The polypeptides are not limited to a specific length. Peptides are included within polypeptides, unless specifically indicated otherwise. The terms neither specify nor exclude post-expression modifications of the polypeptide, for example, glycosylation, acetylation, phosphorylation, and the like, as well as other modifications known in the art, both naturally occurring and non-naturally occurring. Polypeptides of interest in the context of the antibodies of this disclosure include, but are not limited to, polypeptide fragments comprising CDRs which are capable of binding LILRB2 protein expressed by myeloid cells or cancer cells.

The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Fragments are about 5 to 500 amino acids long. For example, fragments can be at least 5, 6, 8, 10, 14, 20, 50, 70, 100, 110, 150, 200, 250, 300, 350, 400, or 450 amino acids long. Polypeptide fragments include immunologically functional fragments of antibodies, including binding domains. In the case of LILRB2 antibody, useful fragments include, but are not limited, to a CDR region, a variable domain of a heavy or light chain, a portion of an antibody chain or just its variable region including two CDRs, and the like.

The term “isolated protein” referred to herein means that a subject protein (1) is free of at least some other proteins with which it would normally be found, (2) is essentially free of other proteins from the same source, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Genomic DNA, cDNA, mRNA, or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antibody) comprises an amino acid sequence in which one or more amino acid residues are inserted into, deleted from and/or substituted into the amino acid sequence relative to another polypeptide sequence. Variants include fusion proteins.

A “derivative” of a polypeptide is a polypeptide (e.g., an antibody) that has been chemically modified in some manner distinct from insertion, deletion, or substitution variants, e.g., via conjugation to another chemical moiety.

The term “antibody” generally includes an immunoglobulin protein that comprises one or more polypeptide chains and that is immunologically functional, including specifically binding to an antigen. In humans, antibodies typically comprise four linked polypeptide chains, a “tetramer” including two identical “heavy” chains and two identical smaller “light” chains. The two heavy chains are each linked to one light chain, are also linked to one another in parallel. The linkages impart a roughly Y-shaped structure to the antibody, such that the linked portions of the heavy chains form the leg of the “Y”, and each light chain (with the portion of the heavy chain to which it is linked) forms an arm of the “Y”. Each arm of the antibody contains an antigen binding site, so the typical antibody can bind two of antigens. In humans there exist five basic types or classes of antibodies, differentiated by the structure of the heavy regions and by their functional purpose: IgG, IgA, IgE, IgD, IgM. Intact antibodies in some classes in humans can differ from the typical tetrameric “Y” structural unit, such as circulating IgM antibodies that contain five such units linked at their bases in a roughly circular array. More detail on antibody structure and function is provided elsewhere herein.

In a typical antibody, each pair or couplet in the tetrameric unit includes one full-length “light” chain (about 25 kDa) and one full-length “heavy” chain (about 50-70 kDa). Each individual immunoglobulin chain is composed of several “immunoglobulin domains,” each consisting of roughly 90 to 110 amino acids and expressing a characteristic folding pattern. These domains are the basic units of which antibody polypeptide chains are composed. The amino-terminal portion of each chain typically includes a variable domain that is responsible for antigen recognition. The carboxy-terminal portion is more conserved evolutionarily than the amino-terminal end of the chain and is referred to as the “constant region” or “C region.”

The term “heavy chain” includes a full-length immunoglobulin heavy chain and fragments thereof having sufficient variable domain sequence to confer binding specificity, either alone or together with a light chain variable domain. Heavy chains are typically classified as mu (p), delta (5), gamma (γ), alpha (a), or epsilon (E) chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. Full-length IgG heavy chains, for example, each contain three C region domains known as CH1, CH2 and CH3, with the CH3 being closest to the carboxy terminus. The antibody products that are provided can have any of these isotypes and subtypes. For example, an LILRB2 antibody product can be an intact antibody of the IgG1 or IgG4 subtype.

The term “light chain” includes a full-length immunoglobulin light chain and fragments thereof having sufficient variable domain sequence to confer binding specificity, either alone or together with a heavy chain variable domain. Human light chains generally are classified as kappa (κ) or lambda (A) light chains. A full-length light chain includes an amino-terminal variable domain (VL) and a carboxy terminal constant domain (CL).

In the light and heavy chains, the variable and constant regions are naturally joined by a “J” region of about twelve or more amino acids, with the heavy chain further including a “D” region of about ten more amino acids. See, e.g., Fundamental Immunology, 2nd ed., Ch. 7 (Paul, ed.) 1989, New York: Raven Press.

Variable domains of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a particular epitope on the target protein (e.g., LILRB2). From N-terminal to C-terminal, naturally occurring light and heavy chain variable regions both typically conform with the following order of these elements: FR1, CDR1, FR2, CDR2, FR3, CDR3 and FR4. Numbering systems have been devised for assigning numbers to amino acids that occupy positions in each of these domains.

The current art utilizes various numbering schemes with different definitions of CDR lengths and positions. For example, the Kabat numbering scheme is based on sequence alignment and uses “variability parameter” of a given amino acid position (the number of different amino acids at a given position divided by the frequency of the most occurring amino acid at that position) to predict CDRs [Kabat et al. in Sequences of Proteins of Immunological Interest, 5th Ed., US Dept. of Health and Human Services, PHS, NIH, NIH Publication no. 91-3242 (1991)]. The Chothia numbering scheme, on the other hand, is a structure-based numbering scheme where antibody crystal structures are aligned as define the loop structures as CDRs [Chothia and Lesk, J Mol Biol. 1987 196:901-17; Chothia et al., Nature. 1989 342:878-83]. The Martin numbering scheme focuses on the structure alignment of different framework regions of unconventional lengths [Martin, “Protein Sequence and Structure Analysis of Antibody Variable Domains.” In: Kontermann and Dübel, eds. Antibody Engineering. Springer; Berlin, Germany: 2014. pp. 33-51]. The ImMunoGeneTics (IMGT) numbering scheme is a standardized numbering system based on alignments of sequences from a complete reference gene database including the whole immunoglobulin superfamily [Lefranc et al., Dev Comp Immunol. 2003 27(1):55-77; (www.imgt.org/IMGTScientificChart/Nomenclature/IMGT-FRCDRdefinition.html)]. The Honneger numbering scheme (AHo) is based on structural alignments of the 3D structure of the variable regions and uses structurally conserved Ca positions to deduce framework and CDR lengths [Honegger et al., J Mol Biol. 2001 309(3):657-70]. One of ordinary skill in the art understands that the definition of a CDR will vary based on the method used.

Some of the antibody products that are provided have the structure typically associated with naturally occurring antibodies. Thus, the term “antibody product” includes an intact antibody of any class or subclass, or a fragment thereof, that can compete with the intact antibody for specific binding to the target antigen, and includes chimeric, humanized, fully human, and bispecific antibodies, as well as other forms. As noted, an intact antibody generally will comprise at least two full-length heavy chains and two full-length light chains, but in some instances can include fewer chains such as antibodies naturally occurring in camelids, which can comprise only heavy chains, and V NAR domains from sharks. Antibody products can be derived solely from a single source, or can be “chimeric,” that is, different portions of the antibody can be derived from two different antibodies. For example, the complementarity determining regions that impart the binding specificity of an antibody, can be derived from a rat or murine source, while the framework region of the variable regions is derived from a different species source, such as a human. In other chimeric forms, the light and heavy variable domains (optionally with a constant domain) can be derived from one species and one or more constant domains from another species. See, e.g., U.S. patent Ser. No. 11/352,444. The antibody products provided can be produced in hybridomas, by recombinant DNA techniques, or by enzymatic or chemical cleavage of intact antibodies. Unless otherwise indicated, the term “antibody product” includes, in addition to antibodies comprising two full-length heavy chains and two full-length light chains (such as IgG antibodies), antibodies of other isotypes, derivatives, variants, and fragments thereof. The antibody products provided include, but are not limited to, monoclonal antibodies, human antibodies, chimeric antibodies, and humanized antibodies. Immunologically functional antibody fragments provided include, but are not limited to, scFv, Fab, Fab′, F(ab′)₂, and domain antibody products.

An “immunologically functional fragment” (or simply “fragment”) of an immunoglobulin, as used herein, refers to a portion of an antibody, comprising a light chain or a heavy chain (or both) and is capable of binding specifically to an antigen, but the light chain or heavy chain (or both) lacks at least some of the amino acids present in a full-length chain. Such fragments are biologically active in that they bind specifically to the target antigen and can compete with intact antibodies for specific binding to a given epitope. Such a fragment will retain at least one CDR present in the full-length light or heavy chain and can comprise a single heavy chain and/or light chain or portion thereof. These biologically active fragments can be produced by recombinant DNA techniques or can be produced by enzymatic or chemical cleavage of intact antibodies. Immunologically functional immunoglobulin fragments include, but are not limited to, Fab, Fab′, F(ab′)₂, Fv, domain antibodies and single-chain antibodies, and can be derived from any mammalian source, including but not limited to human, mouse, rat, camelid or rabbit. It is contemplated further that a functional portion of the inventive antibodies, for example, one or more CDRs, could be covalently bound to a second protein or to a small molecule to create a therapeutic agent directed to a particular target in the body, possessing bifunctional therapeutic properties, or having a prolonged serum half-life.

A “Fab fragment” comprises one light chain (VL+CL) and a portion of a heavy chain that includes the variable domain and the CH1 domain (VH+CH1). The heavy chain of a Fab molecule cannot form a disulfide bond with another heavy chain molecule.

An “Fc” region contains two heavy chain fragments each comprising the CH2 and CH3 domains of an antibody and in some cases the lower hinge region. The two heavy chain fragments are held together by two or more disulfide bonds (typically in the hinge region) and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)₂ molecule.

A “F(ab′)₂ fragment” contains two light chains and two heavy chains each containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable domains from both the heavy and light chains, but lacks the constant domains.

“Single-chain antibodies” are Fv molecules in which the heavy and light chain variable domains have been connected by a flexible linker to form a single polypeptide chain, which forms an antigen-binding region. Single chain antibodies are discussed in detail, for example, in PCT Publication No. WO 88/01649 and U.S. Pat. Nos. 4,946,778 and 5,260,203.

A “domain antibody” is an immunologically functional immunoglobulin fragment containing only the variable domain of a heavy chain or the variable domain of a light chain. In some instances, two or more VH domains are covalently joined with a peptide linker to create a bivalent domain antibody. The two VH domains of a bivalent domain antibody can target the same or different antigens.

A “bivalent antibody” comprises two antigen binding sites. In some instances, the two binding sites have the same antigen specificities. However, bivalent antibodies can be bispecific (see below).

A “multispecific antibody” is one that targets more than one antigen or epitope.

A “bispecific,” “dual-specific” or “bifunctional” antibody is a hybrid antibody having two different antigen binding sites. Bispecific antibodies are a species of multispecific antibody and can be produced by a variety of methods including, but not limited to, fusion of hybridomas or linking of Fab′ fragments. See, e.g., Songsivilai and Lachmann, Clin Exp Immunol 1990 79:315-21; Kostelny et al., J Immunol. 1992 148:1547-53. The two binding sites of a bispecific antibody will bind to two different epitopes, which can reside on the same or different protein targets. A “trispecific” antibody has three different antigen binding sites.

The term “neutralizing antibody” refers to an antibody that binds to a ligand, prevents binding of the ligand to its binding partner and interrupts the biological response that otherwise would result from the ligand binding to its binding partner. In assessing the binding and specificity of an antibody or immunologically functional fragment thereof, an antibody or fragment will substantially inhibit binding of a ligand to its binding partner when an excess of antibody reduces the quantity of binding partner bound to the ligand by at least about 20%, 30%, 40%, 50%, 60%, 70%, 80%, 85%, 90%, 95%, 97%, 99% or more (as measured in an in vitro competitive binding assay). In the case of antibody products that bind to LILRB2, a neutralizing antibody product will diminish the ability of LILRB2 to bind to one or more of its ligands thereby inhibiting LILRB2 activity (e.g., as shown in the Examples herein).

The term “competition” when used in the context of antibody products that compete for the same epitope means competition between antibodies is determined by an assay in which the antibody product under test prevents or inhibits specific binding of a reference antibody product to a common antigen (e.g., LILRB2 or a fragment thereof). Numerous types of competitive binding assays can be used, for example: solid phase direct or indirect radioimmunoassay (RIA), solid phase direct or indirect enzyme immunoassay (EIA), sandwich competition assay [e.g., Stahli et al., Methods Enzymol. 1983 9:242-53]; solid phase direct biotin-avidin EIA [e.g., Kirkland et al., J Immunol 1986 137:3614-9]; solid phase direct labeled assay, solid phase direct labeled sandwich assay [e.g., Harlow and Lane, Antibodies, A Laboratory Manual, Cold Spring Harbor Press (1988)]; solid phase direct label RIA using I-125 label [e.g., Morel et al., Molec Immunol 1988 25:7-15]; solid phase direct biotin-avidin EIA [e.g., Cheung et al., Virology. 1990 176:546-52]; and direct labeled RIA [Moldenhauer et al., Scand J Immunol 1990 32:77-82]. Typically, such an assay involves the use of purified antigen bound to a solid surface or cells bearing either of these, an unlabeled test antibody and a labeled reference antibody. Competitive inhibition is measured by determining the amount of label bound to the solid surface or cells in the presence of the test antibody. Usually, the test antibody is present in excess. Antibodies identified by competition assay (competing antibodies) include antibodies binding to the same epitope as the reference antibody and antibodies binding to an epitope sufficiently proximal to the epitope bound by the reference antibody for steric hindrance to occur. Usually, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more by a selective binding agent, such as an antibody, and additionally capable of being used in an animal to produce antibodies capable of binding to that antigen. An antigen can possess one or more epitopes that are capable of interacting with different antibodies.

The term “epitope” includes any determinant capable of specifically binding to an antibody or to a T-cell receptor. An epitope is a region of an antigen that is bound by an antibody that specifically targets that antigen, and when the antigen is a protein, includes specific amino acids that directly contact the antibody. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of proteins and/or macromolecules.

An antibody product “specifically binds” its target antigen when the dissociation constant (K_{d}) is less than 100 nM. The antibody specifically binds antigen with “high affinity” when the K_{d} is less than 10 nM and with “very high affinity” when the K_{d} is less than 0.5 nM. The antibody product can have a K_{d} in the range from about 0.5 nM to about 500 nM. The antibody product can have a K_{d} in the range from about 100 to about 500 nM. One of skill in the art will recognize that specifically binding does not mean exclusive binding, rather it allows for some degree of non-specific binding as is typical in biological reactions between groups with affinity to one another.

As used herein, the term “affinity” refers to the equilibrium constant for the reversible binding of two agents and can be expressed as the equilibrium dissociation constant, K_{D}, a calculated ratio of the dissociation constant and the association constant (K_{off}/Kₒₙ), between the antibody and its antigen. Affinity can also be expressed as the association constant, K_{A}, which is the reciprocal of K_{D}. The antibody products disclosed herein exhibit binding affinity as measured by K_{D} for human LILRB2 and for human LILRB1 in the range of 10⁻⁴ M or less, or ranging down to 10⁻¹⁶ M or lower, (e.g., about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶ M or less). Antibodies described herein can specifically bind to a human LILRB2 polypeptide or to a human LILRB1 polypeptide with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Methods for determining the affinity of two molecules are well known in the art and include, for example, equilibrium dialysis, surface plasmon resonance (SPR), bio-layer interferometry (BLI), and the like.

As used herein, an antibody product is said to be “immunospecific” or “specific” for, or to “specifically bind” when that an antibody product forms a complex with an antigen that is relatively stable under physiologic conditions. The terms “preferentially binds” or “specifically binds” mean that the antibodies or fragments thereof bind to an epitope with greater affinity than it binds unrelated amino acid sequences, and, if cross-reactive to other polypeptides containing the epitope, are not toxic at the levels at which they are formulated for administration to human use. Such affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody product for unrelated amino acid sequences. The terms are also applicable where for example, an antibody product is specific for a particular epitope that is carried by more than one antigen, in which case the antibody or antigen-binding fragment thereof carrying the antigen-binding domain will be able to specifically bind to the epitope found in the different antigens.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, Ed.), 1988, New York: Oxford University Press; Biocomputing Informatics and Genome Projects, (Smith, ed.), 1993, New York: Academic Press; Computer Analysis of Sequence Data, Part I, (Griffin and Griffin, eds.), 1994, New Jersey: Humana Press; Sequence Analysis in Molecular Biology, (von Heinje), 1987, New York: Academic Press; Sequence Analysis Primer, (Gribskov and Devereux, eds.), 1991, New York: M. Stockton Press; and Carillo et al., SIAM J Applied Math. 1988 48(5):1073-82.

In calculating percent identity, the sequences being compared are aligned in a way that gives the largest match between the sequences. An exemplary computer program used to determine percent identity is the GCG program package, which includes GAP (Devereux et al., Nucl Acid Res. 1984 12:387-95; Genetics Computer Group, University of Wisconsin, Madison, Wisc.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3.times. the average diagonal, in which the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix [e.g., Dayhoff et al., Atlas of Protein Sequence and Structure, 5:345-352 (1978) for the PAM 250 comparison matrix; Henikoff et al., Proc Natl Acad Sci USA. 1992 89:10915-9 for the BLOSUM 62 comparison matrix] can also be used by the algorithm.

Recommended parameters for determining percent identity for polypeptides or nucleotide sequences using the GAP program are the following: Algorithm: Needleman et al., J Mol Biol. 1970 48:443-53; Comparison matrix: BLOSUM 62 from Henikoff et al., 1992, supra; Gap Penalty: 12 (but with no penalty for end gaps); Gap Length Penalty: 4; Threshold of Similarity: 0.

Certain alignment schemes for aligning two amino acid sequences can result in matching of only a short region of the two sequences, and this small aligned region can have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

Other exemplary programs that compare and align pairs of sequences include, but are not limited to, ALIGN (Myers and Miller, Comput Appl Biosci. 1988 4(1):11-7); FASTA (Pearson and Lipman, Proc Natl Acad Sci USA. 1988 85(8):2444-8; Pearson, Methods Enzymol. 1990 183:63-98); and gapped BLAST (Altschul et al., Nucleic Acids Res 1997 25(17):3389-402), BLASTP, BLASTN, or GCG (Devereux et al., Nucleic Acids Res. 1984 12(1 Pt 1):387-95).

“Amino acid” includes its normal meaning in the art. The twenty naturally-occurring amino acids and their abbreviations follow conventional usage. See Immunology—A Synthesis, 2nd ed. (Golub and Gren, Eds.), Sinauer Associates: Sunderland, Mass. (1991). Stereoisomers (e.g., D-amino acids) of the twenty conventional amino acids, unnatural amino acids such as α-, α-disubstituted amino acids, N-alkyl amino acids, and other unconventional amino acids can be suitable components. Examples of unconventional amino acids include: 4-hydroxyproline, gamma-carboxyglutamate, ε-N,N,N-trimethyllysine, ε-N-acetyllysine, O-phosphoserine, N-acetylserine, N-formylmethionine, 3-methylhistidine 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Table 10 below sets out heavy and light chain variable domains for an LILRB2-specific antibody designated B32A, and corresponding full-length heavy (hIgG1 and hIgG4) and light (kappa) chains provided herein. IMGT and Kabat CDRs are presented in Table 11. CDRs of the two variable domains are indicated: IMGT in double underline; Kabat in bold.

TABLE 10
Variable Regions and Full Length

| SEQ ID NO: | Name | Sequence |
|---|---|---|
| 1 | B2A-VH | QSVEESGGRLVTPGTPLTLTCRVSGFSLSTYAMSWVRQAPGKGLEWIGWIY |
| | | NDGSTYYAAWAKGRFTFSKTSTTVDLRITSPTPEDTATYFCAREGDYIGYF |
| | | NIWGQGTLVTVSS |
| 2 | B2A-VL | DVVMTQTPSSVSAALGGTVTIKCQASQSIGSNLAWYQLKPGQRPKLLIYAA |
| | | ATLASGGSSRFKGSGSGTDFTLTISDLECADAATYYCQSYVSGSSDVAFGG |
| | | GTEVVVKRT |
| 3 | B2A-HC1 | QSVEESGGRLVTPGTPLTLTCRVSGFSLSTYAMSWVRQAPGKGLEWIGWIY |
| | (hIgG1) | NDGSTYYAAWAKGRFTFSKTSTTVDLRITSPTPEDTATYFCAREGDYIGYF |
| | | NIWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKP |
| | | SNTKVDKRVEPKSCDKTHTCPPCPAPELLGGPSVFLFPPKPKDTLMISRTP |
| | | EVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| 4 | B2A-HC4 | QSVEESGGRLVTPGTPLTLTCRVSGFSLSTYAMSWVRQAPGKGLEWIGWIY |
| | (hIgG4) | NDGSTYYAAWAKGRFTFSKTSTTVDLRITSPTPEDTATYFCAREGDYIGYF |
| | | NIWGQGTLVTVSSASTKGPSVFPLAPCSRSTSESTAALGCLVKDYFPEPVT |
| | | VSWNSGALTSGVHTFPAVLQSSGLYSLSSVVTVPSSSLGTKTYTCNVDHKP |
| | | SNTKVDKRVESKYGPPCPPCPAPEFLGGPSVFLFPPKPKDTLMISRTPEVT |
| | | CVVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYSRLTVD |
| | | KSRWQEGNVFSCSVMHEALHNHYTQKSLSLSLGK |
| 5 | B2A-LCK | DVVMTQTPSSVSAALGGTVTIKCQASQSIGSNLAWYQLKPGQRPKLLIYAA |
| | (kappa) | ATLASGGSSRFKGSGSGTDFTLTISDLECADAATYYCQSYVSGSSDVAFGG |
| | | GTEVVVKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGLS |
| | | SPVTKSFNRGEC |

TABLE 11
Complementarity Determining Regions

| SEQ ID NO: | Name | Sequence |
|---|---|---|
| 16 | CDR-H1 IMGT | GFSLSTYAMS |
| 17 | CDR-H2 IMGT | IYNDGST |
| 18 | CDR-H3 IMGT | AREGDYIGYFNI |
| 19 | CDR-L1 IMGT | SIGSN |
| 20 | CDR-L2 IMGT | AAA |
| 21 | CDR-L3 IMGT | QSYVSGSSDVA |
| 22 | CDR-H1 Kabat | TYAMS |
| 23 | CDR-H2 Kabat | WIYNDGSTYYAAWAKG |
| 24 | CDR-H3 Kabat | EGDYIGYFNI |
| 25 | CDR-L1 Kabat | QASQSIGSNLA |
| 26 | CDR-L2 Kabat | AAATLAS |
| 27 | CDR-L3 Kabat | QSYVSGSSDVA |

Humanized variants of the B32A antibody were prepared as described in the Examples. Variable domains of representative humanized heavy and light chains are presented in Table 12.

TABLE 12

| SEQ ID NO: | Name | Sequence |
|---|---|---|
| 6 | B2H-VH1 | QVQLEESGGRVVQPGRSLRLSCAASGFSLSTYAMSWVRQAPGKGLEWIGW |
| | | IYNDGSTYYAAWAKGRFTISKDTSKTTVFLQMNSLKPDDTATYFCAREGD |
| | | YIGYFNIWGQGTLVTVSS |
| 7 | B2H-VH2 | EVQLVESGGDLATPGGSLRLSCAVSGFSLSTYAMSWVRQAPGKGLEWVGW |
| | | IYNDGSTYYAAWAKGRFTISKDTSKTTVYLQMTSLRAEDTALYFCAREGD |
| | | YIGYFNIWGQGTLVTVSS |
| 8 | B2H-VH3 | QVQLVESGGRVVQPGTPLRLSCAASGFSLSTYAMSWVRQAPGKGLEWVAW |
| | | IYNDGSTYYAAWAKGRFTISRDTSKNTLYLQTNSLKPDDTAVYYCAREGD |
| | | YIGYFNIWGQGTLVTVSS |
| 9 | B2H-VH4 | EVQLLESGGRLVQPGTSLRLSCAVSGFSLSTYAMSWVRQAPGKGLEWIGW |
| | | IYNDGSTYYAAWAKGRFTISKDTSKTTLYLQMNSLTVEDTATYFCAREGD |
| | | YIGYFNIWGQGTLVTVSS |
| 10 | B2H-VH5 | EVQLLESGGGLVQPGTSLTLSCAASGFSLSTYAMSWVRQAPGKGLEWIGW |
| | | IYNDGSTYYAAWAKGRFTISKDTSKTTLYLQITSLRAEDTATYFCAREGD |
| | | YIGYFNIWGQGTLVTVSS |
| 11 | B2H-VL1 | DIQMTQSPSSLSASVGDRVTITCQASQSIGSNLAWYQLKPGQRPKLLIYA |
| | | AATLASGVPSRFKGSGSGTDFTLTISSLQSEDAATYYCQSYVSGSSDVAF |
| | | GGGTEVVVK |
| 12 | B2H-VL2 | DVVMTQSPSSVSASLGDRVTITCQASQSIGSNLAWYQQKPGQAPKLLIYA |
| | | AATLASGVPSRFKGSGSGTDFTLTITSLQPADFATYYCQSYVSGSSDVAF |
| | | GGGTKVEIK |
| 13 | B2H-VL3 | DIEMTQSPSSVSASVGDRVTLTCQASQSIGSNLAWYQQKPGQAPKLLIYA |
| | | AATLASGVPSRFKGSGSGTDFTLTISSLQPEDFATYYCQSYVSGSSDVAF |
| | | GGGTEVVVK |
| 14 | B2H-VL4 | DIQMTQSPSSVSASVGDRVTITCQASQSIGSNLAWYQQKPGKAPKLLIYA |
| | | AATLASGVPLRFSGSGSGTDFTLTISNLQPEDSATYYCQSYVSGSSDVAF |
| | | GGGTKVEIK |
| 15 | B2H-VL5 | DVVMTQTPSSVSAALGGTVTIKCQASQSIGSNLAWYQLKPGQRPKLLIYA |
| | | AATLASGGSSRFKGSGSGTDFTLTISSLEPADAATYYCQSYVSGSSDVAF |
| | | GGGTEVVVK |

The skilled artisan recognizes that antibody products, such as full-length intact antibodies and LILRB2-binding antibody fragments, can be prepared based the heavy chain and light chain variable domains given in Tables 10 and 12, or on the CDRs given in Table 11. To demonstrate, as described in the Examples, in addition to the full-length antibody chains shown in Table 10, full-length IgG1 or IgG4 heavy chains were prepared comprising the VH variable domains shown in Table 12 (SEQ ID NOs: 6-10), in combination with kappa light chains comprising the variable domains shown in Table 12 (SEQ ID NOs: 11-15). Full length IgG1 heavy chain sequences are presented as SEQ ID NOS: 28-32. Full length IgG4 heavy chain sequences are presented as SEQ ID NOS: 33-37. Full length kappa light chains sequences are presented as SEQ ID NOS: 38-42. LILRB2-binding antibody products were prepared using all combinations of the heavy and light chains, and the testing of some of these in various non-clinical assays is described in the Examples.

Antibody products can comprise a light chain variable domain comprising a sequence of amino acids that differs from the sequence of a light chain variable domain described herein at only 1, 2, 3, 4, 5, 6, 7, 8, 9, 10, 11, 12, 13, 14, or 15 amino acid residues, in which each such sequence difference is independently either a deletion, insertion or substitution of one amino acid. The light chain variable region in some antibodies comprises a sequence of amino acids that has at least 70%, 75%, 80%, 85%, 90%, 95%, 96%, 97%, 98%, or 99% sequence identity to an amino acid sequence of a light chain variable region in Table 10 or in Table 12.

Antibody products can comprise a heavy chain variable domain comprising a sequence of amino acids that differs from the sequence of a heavy chain variable domain provided herein at only 1, 2, 3, 4, 5, 6, 7, 8, 9, 10, 11, 12, 13, 14 or 15 amino acid residues, in which each such sequence difference is independently either a deletion, insertion or substitution of one amino acid. The heavy chain variable region in some antibodies comprises a sequence of amino acids that has at least 70%, 75%, 80%, 85%, 90%, 95%, 96%, 97%, 98% or 99% sequence identity to the amino acid sequence of the heavy chain variable region in Table 10 or in Table 12. Still other antibody products include variant forms of a variant light chain and a variant heavy chain as just described.

The antibody products that are provided can include one, two, three, four, five or all six CDRs. Some antibody products include both the light chain CDR3 and the heavy chain CDR3. Certain antibody products have variant forms of the CDRs, with one or more (i.e., 2, 3, 4, 5 or 6) of the CDRs each having at least 80%, 85%, 90%, 95%, 96%, 97%, 98% or 99% sequence identity to a CDR sequence. For example, the antibody product can include both a light chain CDR3 and a heavy chain CDR3 that each have at least 80%, 85%, 90%, 95%, 96%, 97%, 98% or 99% sequence identity to the light chain CDR3 sequence and the heavy chain CDR3, respectively. The CDR sequences of the antibody products that are provided can also differ from the CDR sequences in Table 10 or in Table 12 such that the amino acid sequence for any given CDR differs from the sequence listed in Table 10 or in Table 12 by no more than one, two, three, four or five amino acid residues. Differences from the listed sequences are conservative substitutions.

When an antibody product is said to bind an epitope of LILRB2, what is meant is that the antibody product specifically binds to a region of LILRB2 specified by certain residues (e.g., one or more specified segments of LILRB2 protein). In binding to an LILRB2 epitope, an antibody does not necessarily contact every residue within the LILRB2 peptide comprising the specified residues. Nor does every single amino acid substitution or deletion within the LILRB2 peptide necessarily significantly affect binding affinity. Exact epitope specificity of an antibody can be determined in variety of ways. One approach, for example, involves testing a collection of overlapping peptides of about fifteen amino acids spanning the sequence of LILRB2 and differing in increments of a small number of amino acids (e.g., three amino acids). The peptides are immobilized within the wells of a microtiter dish. Immobilization can be achieved by biotinylating one terminus of the peptides. Optionally, different samples of the same peptide can be biotinylated at the N and C terminus and immobilized in separate wells for purposes of comparison. This is useful for identifying end-specific antibodies. Optionally, additional peptides can be included terminating at a particular amino acid of interest. This approach is useful for identifying end-specific antibodies to internal fragments of LILRB2. An antibody product is screened for specific binding to each of the various peptides. The epitope is defined as occurring within a segment of amino acids that is common to all peptides to which the antibody shows specific binding.

Antibody products that compete with one of the exemplified antibodies for specific binding to LILRB2 are also provided. Such antibody products can also bind to the same epitope as one of the exemplified antibodies. Antibody products that compete with or bind to the same epitope as the exemplified antibody or fragment are expected to show similar functional properties. The exemplified antibody products include those with the heavy and light chains, variable domains and CDRs provided in Table 10s Table 11, or Table 12. Competing antibody products can include those that bind to the epitope described in the section on antibodies and epitopes above.

The antibody products provided include monoclonal antibodies that bind to LILRB2. Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7. and S194/5XXO Bul; examples of cell lines used in rat fusions include R210.RCY3, Y3-Ag 1.2.3, IR983F, and 4B210. Other cell lines useful for cell fusions are U-266, GM1500-GRG2, LICR-LON-HMy2, and UC729-6.

In some instances, a hybridoma cell line is produced by immunizing an animal (e.g., a transgenic animal having human immunoglobulin sequences) with a LILRB2 immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds a LILRB2 polypeptide. Such hybridoma cell lines, and LILRB2 monoclonal antibodies produced by them, are provided herein.

Monoclonal antibodies secreted by a hybridoma cell line can be purified using any useful technique known in the antibody arts. Hybridomas or monoclonal antibodies can be further screened to identify monoclonal antibodies with particular properties. Examples of such screens are provided in the Examples below.

Chimeric and humanized antibodies based upon the foregoing sequences are also provided. Monoclonal antibodies for use as therapeutic agents can be modified in various ways prior to use. One example is a “chimeric” antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., Proc Natl Acad Sci USA. 1985 81:6851-5. CDR grafting is described, for example, in U.S. Pat. Nos. 6,180,370, 5,693,762, 5,693,761, 5,585,089, and 5,530,101.

Generally, the goal of making a chimeric antibody is to create a chimera in which the number of amino acids from the intended patient species is maximized. One example is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the V region or selected CDRs from a rodent antibody often are grafted into a human antibody, replacing the naturally occurring V regions or CDRs of the human antibody.

“Humanized” antibody products are provided. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region with the corresponding regions of a human antibody [e.g., U.S. Pat. Nos. 5,585,089, and 5,693,762; Jones et al., Nature. 1986 321:522-5; Riechmann et al., Nature. 1988 332:323-7; Verhoeyen et al., Science. 1988 239:1534-6]. Constant regions from species other than human can be used along with the human variable region(s) to produce hybrid antibodies.

Fully human antibodies are also provided. Methods are known for making fully human antibodies specific for a given antigen without exposing human beings to the antigen (“fully human antibodies”). One means for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (MAbs) in mouse, an animal that can be immunized with any desirable antigen. Using fully human antibodies can minimize the immunogenic and allergic responses that can sometimes be caused by administering mouse or mouse-derivatized monoclonal antibodies to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc Natl Acad Sci USA. 1993 90:2551-5; Jakobovits et al., Nature. 1993 362:255-8; and Bruggemann et al., Year Immunol. 1993 7:33-40. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,545,807; 6,300,129; 6,255,458; 5,877,397; 5,874,299 and 5,545,806; in PCT Publication Nos. WO 91/10741, WO 90/04036, and in EPO Publication No. EP 546073B1. The transgenic mice, referred to herein as “HuMab” mice, contain a human immunoglobulin gene minilocus that encodes unrearranged human heavy (g and gamma) and kappa light chain immunoglobulin sequences, together with targeted mutations that inactivate the endogenous gamma and kappa chain loci (Lonberg et al., Nature. 1994 368:856-9). Accordingly, the aforementioned mice exhibit reduced expression of mouse IgM or kappa and in response to immunization the introduced human heavy and light chain transgenes undergo class switching and somatic mutation to generate high affinity human IgG kappa monoclonal antibodies. An exemplary mouse with the entire human immunoglobulin locus in its germline is the XenoMouse (Abgenix). Another is the VelociImmune mouse (Regeneron Pharmaceuticals). Others are the RenMab mouse and the RenLite mouse (Biocytogen), and, more recently the AlivaMab Mouse (Ablexis).

Using hybridoma technology, antigen-specific human monoclonal antibodies with the desired specificity can be produced and selected from the transgenic mice such as those described above. Such antibodies can be cloned and expressed using a suitable vector and host cell, or the antibodies can be harvested from cultured hybridoma cells.

Fully human antibodies can also be derived from phage-display libraries (as disclosed in Hoogenboom and Winter, J Mol Biol. 1992 227(2):381-8; and Marks et al., J Mol Biol. 1991 222:581-97. Phage display techniques mimic immune selection through the display of antibody repertoires on the surface of filamentous bacteriophage, and subsequent selection of phage by their binding to an antigen of choice. One such technique is described in PCT Publication No. WO 99/10494, which describes the isolation of high affinity and functional agonistic antibodies for c-Mpl and MuSK receptors using such an approach.

Single chain antibodies are provided. Single chain antibodies are formed by linking heavy and light chain variable domain (Fv region) fragments (such as those shown in Table 10 or in Table 12) via an amino acid bridge (short peptide linker), resulting in a single polypeptide chain. Such single-chain Fvs (scFvs) can be prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains. Techniques developed for the production of single chain antibodies include those described in U.S. Pat. No. 4,946,778; Bird et al., Science. 1988 242:423-6; Huston et al., Proc Natl Acad Sci USA. 1988 85:5879-83; Ward et al., Nature. 1989 334:544-6; and de Graaf et al., Methods Mol Biol. 2002 178:379-87. A “diabody” is a dimer of scFV.

Antibodies provided herein that are of one subclass can be changed to antibodies from a different subclass using subclass switching methods. For example, the variable domains depicted in Table 10 or in Table 12 can be attached to constant domains of any desired Ig subtype. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques can be employed. Cloned DNA encoding particular antibody polypeptides can be employed in such procedures, e.g., DNA encoding the constant domain of an antibody of the desired isotype. See, e.g., Lantto et al., Methods Mol Biol. 2002 178:303-16. Accordingly, the antibodies that are provided include a desired isotype (for example, IgA, IgG1, IgG2, IgG3, IgG4, IgE, and IgD).

Antibody products provided can include one or more of the CDRs of any of the heavy chain variable domains exemplified herein, where such CDRs are determined according to IMGT, Kabat or other method: (i) a CDR-H1 with at least 80% sequence identity to the CDR-H1 of SEQ ID NO: 1, 6, 7, 8, 9, or 10; (ii) a CDR-H2 with at least 80% sequence identity to the CDR-H2 of SEQ ID NO: SEQ ID NO: 1, 6, 7, 8, 9, or 10; and (iii) a CDR-H3 with at least 80% sequence identity to the CDR-H3 of SEQ ID NO: SEQ ID NO: 1, 6, 7, 8, 9, or 10. Antibody products provided can include one or more of the CDRs of any of the light chain variable domains exemplified herein, where such CDRs are determined according to IMGT, Kabat or other method: (i) a CDR-L1 with at least 80% sequence identity to the CDR-L1 of SEQ ID NO: 2, 11, 12, 13, 14, or 15; (ii) a CDR-L2 with at least 80% sequence identity to the CDR-L2 of SEQ ID NO: SEQ ID NO: 2, 11, 12, 13, 14, or 15; and (iii) a CDR-L3 with at least 80% sequence identity to the CDR-L3 of SEQ ID NO: SEQ ID NO: 2, 11, 12, 13, 14, or 15. In some embodiments, the CDRs will have at least 85%, at least 90%, at least 95%, or least 99% identity to the determined CDR sequences. Antibody products can include one, two, three, four, five or all six of the foregoing CDRs, as long as they specifically bind hLILRB2.

Antibody products provided can include one or more of the following exemplary heavy chain IMGT CDRs: (i) a CDR-H1 with at least 80% sequence identity to SEQ ID NO: 16; (ii) a CDR-H2 with at least 80% sequence identity to SEQ ID NO: 17; and (iii) a CDR-H3 with at least 80% sequence identity to SEQ ID NO: 18. Antibody products provided can include one or more of the following light chain CDRs: (i) a CDR-L1 with at least 80% sequence identity to SEQ ID NO: 19; (ii) a CDR-L2 with at least 80% sequence identity to SEQ ID NO: 20; and (iii) a CDR-L3 with at least 80% sequence identity to SEQ ID NO: 21. In some embodiments, the CDRs will have at least 85%, at least 90%, at least 95%, or least 99% identity to the specified CDR sequences. Antibody products can include one, two, three, four, five or all six of the foregoing CDRs, as long as they specifically bind hLILRB2.

Antibody products provided can include one or more of the following exemplary heavy chain Kabat CDRs: (i) a CDR-H1 with at least 80% sequence identity to SEQ ID NO: 22; (ii) a CDR-H2 with at least 80% sequence identity to SEQ ID NO: 23; and (iii) a CDR-H3 with at least 80% sequence identity to SEQ ID NO: 24. Antibody products provided can include one or more of the following light chain CDRs: (i) a CDR-L1 with at least 80% sequence identity to SEQ ID NO: 25; (ii) a CDR-L2 with at least 80% sequence identity to SEQ ID NO: 26; and (iii) a CDR-L3 with at least 80% sequence identity to SEQ ID NO: 27. In some embodiments, the CDRs will have at least 85%, at least 90%, at least 95%, or least 99% identity to the specified CDR sequences. Antibody products can include one, two, three, four, five or all six of the foregoing CDRs, as long as they specifically bind hLILRB2.

Antibody products provided can include (a) a heavy chain variable region having 80%, 85%, 90%, 92%, 95%, 96%, 97%, 98%, 99% or greater sequence identity with SEQ ID NO: 1, 6, 7, 8, 9, or 10; (b) a light chain variable region having at least 80%, 85%, 90%, 92%, 95%, 96%, 97%, 98%, 99% or greater sequence identity with SEQ ID NOs: 2, 11, 12, 13, 14, or 15; or (c) a heavy chain variable region of (a) and a light chain variable region of (b).

Other antibody products provided compete with an antibody such as those described above for specific binding to a LILRB2 polypeptide. For example, antibody products are provided that compete with an antibody that consists of two identical heavy chains and two identical light chains, in which the heavy chains comprise SEQ ID NO: 1, 6, 7, 8, 9, or 10, and the light chains comprise SEQ ID NO: 2, 11, 12, 13, 14, or 15.

LILRB2 antibody product are provided that have a half-life of at least one day in vitro or in vivo (e.g., when administered to a human subject). The antibody product can have a half-life of at least three days. The antibody product can have a half-life of four days or longer. The antibody product can have a half-life of eight days or longer.

### Variants

Variant forms of LILRB2 antibody products disclosed herein (e.g., variant forms of antibody products having sequences listed in Tables 10 and 12) are provided. For example, antibody products can have one or more conservative amino acid substitutions in one or more of the heavy or light chain variable regions, or CDRs, listed in Tables 10 and 12.

Naturally occurring amino acids can be divided into classes based on common side chain properties: 1) hydrophobic: norleucine, Met, Ala, Val, Leu, IIe; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe. Conservative amino acid substitutions can involve exchange of a member of one of these classes with another member of the same class. Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions can involve the exchange of a member of one of the above classes for a member from another class. Such substituted residues can be introduced into regions of the antibody product that are homologous with human antibodies, or into the non-homologous regions of the molecule.

In making such changes, the hydropathic index of amino acids can be considered. The hydropathic profile of a protein is calculated by assigning each amino acid a numerical value (“hydropathy index”) and then repetitively averaging these values along the peptide chain. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic profile in conferring interactive biological function on a protein is understood in the art [e.g., Kyte and Doolittle, J Mol Biol. 1982 157:105-31]. It is known that certain amino acids can be substituted for other amino acids having a similar hydropathic index or score and still retain a similar biological activity. In making changes based upon the hydropathic index, the substitution of amino acids whose hydropathic indices are within ±0.2 can be made. The substitution of amino acids whose hydropathic indices are within ±0.1 can also be made. The substitution of amino acids whose hydropathic indices are within ±0.5 can also be made.

It is also understood in the art that the substitution of like residues in amino acid sequences can be made effectively on the basis of relative hydrophilicity or hydrophobicity of the residues, particularly where the biologically functional protein or peptide thereby created is intended for use in immunological molecules, as in the present case. The greatest local average hydropathic character of a protein, as governed by the hydrophilicity of its adjacent amino acids, can correlate with its immunogenicity and antigen-binding or immunogenicity, that is, with a biological property of the protein.

Various methods are known for estimating the hydrophilicity or hydrophobicity of amino acid residues in proteins. A comparative survey of such methods is given at Biswas et al., J Chromatogr A. 1000(1-2):637-55. Hopp and Woods (Mol Immunol. 1983 20(4):483-9) assigned the hydrophilicity values amino acid residues: arginine (+3.0); lysine (+3.0); aspartate (+3.0±0.1); glutamate (+3.0±0.1); serine (+0.3); asparagine (+0.2); glutamine (+0.2); glycine (0); threonine (−0.4); proline (−0.5±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In this ranking system, more hydrophilic residues are assigned positive values, and less hydrophilic residues negative. In making changes based upon similar hydrophilicity values, the substitution of amino acids whose hydrophilicity values are within ±0.2 is included, otherwise those which are within ±0.1 are included or those within ±0.5 are included. In some instances, one can also identify epitopes from primary amino acid sequences on the basis of hydrophilicity. These regions are also referred to as “epitopic core regions.”

A skilled artisan will be able to determine suitable variants of polypeptides as set forth herein using well-known techniques. One skilled in the art can identify suitable areas of the molecule that can be changed without destroying activity by targeting regions not believed to be important for activity. The skilled artisan also will be able to identify residues and portions of the molecules that are conserved among similar polypeptides. Even areas that can be important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar polypeptides. In view of such information, one skilled in the art can predict the alignment of amino acid residues of an antibody with respect to its three-dimensional structure. One skilled in the art can choose not to make radical changes to amino acid residues predicted to be on the surface of the protein, since such residues can be involved in important interactions with other molecules. Moreover, one skilled in the art can generate test variants containing a single amino acid substitution at each desired amino acid residue. These variants can then be screened using assays for LILRB2 activity (see Examples below), thus yielding information regarding which amino acids can be changed and which must not be changed. In other words, based on information gathered from such routine experiments, one skilled in the art can readily determine the amino acid positions where further substitutions should be avoided either alone or in combination with other mutations.

Substantial modifications in the functional and/or biochemical characteristics of the antibody products described herein can be achieved by creating substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulkiness of the side chain. A “conservative amino acid substitution” can involve a substitution of a native amino acid residue with a normative residue that has little or no effect on the polarity or charge of the amino acid residue at that position. Furthermore, any native residue in the polypeptide can also be substituted with alanine, as has been previously described for alanine scanning mutagenesis.

Amino acid substitutions (whether conservative or non-conservative) of the subject antibodies can be implemented by those skilled in the art by applying routine techniques. Amino acid substitutions include, but are not limited to, substitutions that: (1) reduce susceptibility to proteolysis, (2) reduce susceptibility to oxidation, (3) alter binding affinity for forming protein complexes, (4) alter ligand or antigen binding affinities, and/or (4) confer or modify other physicochemical or functional properties on such polypeptides. For example, single or multiple amino acid substitutions (e.g., conservative amino acid substitutions) can be made in the naturally-occurring sequence. Substitutions can be made in that portion of the antibody that lies outside the domain(s) forming intermolecular contacts). Conservative amino acid substitutions can be used that do not substantially change the structural characteristics of the parent sequence (e.g., one or more replacement amino acids that do not disrupt the secondary structure that characterizes the parent or native antibody). Examples of art-recognized polypeptide secondary and tertiary structures are described in Proteins, Structures and Molecular Principles (Creighton, ed.), 1984, New York: W. H. Freeman and Company; Introduction to Protein Structure (Branden and Tooze, eds.), 1991, New York: Garland Publishing; and Thornton et al., Nature. 1991 354(6349):105-6, which are each incorporated herein by reference.

Glycosylation variants of the antibody products are provided in which the number and/or type of glycosylation site(s) has been altered compared to the amino acid sequences of the parent polypeptide. Antibody product variants can comprise a greater or a lesser number of N-linked glycosylation sites than the native antibody. An N-linked glycosylation site is characterized by the sequence: Asn-X-Ser or Asn-X-Thr, in which the amino acid residue designated as X can be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. For example, one or more new N-linked sites are created. Antibodies typically have a N-linked glycosylation site in the Fc region.

Additional antibody product variants include cysteine variants in which one or more cysteine residues in the parent or native amino acid sequence are deleted from or substituted with another amino acid (e.g., serine). Cysteine variants are useful, inter alia when antibodies must be refolded into a biologically active conformation. Cysteine variants can have fewer cysteine residues than the native antibody, and typically have an even number to minimize interactions resulting from unpaired cysteines.

### Effector Functions

Antibody structure affects the role that the antibody plays in the immune system and the effects that the antibody can induce or influence. See, e.g., Vidarsson et al., Front Immunol. 2014 5(Art. 5):1-17. Examples of antibody effector functions include: C1q binding and complement dependent cytotoxicity; Fc receptor binding; antibody-dependent cell-mediated cytotoxicity (ADCC); phagocytosis; down-regulation of cell surface receptors (e.g., B cell receptor); and B cell activation. Typically, the Fc-mediated functions involve binding of the Fc portion of an antibody by specialized receptor molecules, “Fc receptors” or “FcR,” expressed by the cell whose function is to be affected.

IgG is considered the most versatile immunoglobulin because it carries out all of the functions of immunoglobulin molecules in some embodiments. IgG is the major Ig in serum, and the only class of Ig that crosses the placenta. IgG also fixes complement, although the IgG4 subclass does not. Macrophages, monocytes, polymorphonuclear leukocytes (PMNs), and some lymphocytes have receptors for the Fc region of IgG. Not all subclasses bind equally well: IgG2 and IgG4 do not bind to Fc receptors. A consequence of binding to the Fc receptors on PMNs, monocytes, and macrophages is that the cell now internalizes the antigen better in some cases. IgG is an opsonin that enhances phagocytosis. Binding of IgG to Fc receptors on other types of cells results in the activation of other functions.

In certain embodiments, the FcR is a native sequence human FcR. Moreover, a preferred FcR is one that binds an IgG antibody (a gamma (“γ”) receptor) and includes receptors of the FcγRI (CD64), FcγRII (CD32), and FcγRIII (CD16) subclasses, including allelic variants and alternatively spliced forms of these receptors FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII, and FcγRIII. To assess ADCC activity of a molecule of interest, an in vitro ADCC assay is performed in some embodiments. Useful effector cells for such assays include peripheral blood mononuclear cells (PBMC) and Natural Killer (NK) cells.

Alternatively, or additionally, in some embodiments, ADCC activity of the molecule of interest is assessed in vivo, e.g., in an animal model.

In some embodiments, the antibodies of the disclosure bind to a surface membrane protein of and are internalized by M2-like macrophages. This internalization process is believed to be involved in the observed alteration of the functional immunosuppressive characteristics of these cells, i.e., the differentiation of the cells from M2 status to subtly activated state, without killing them or inhibiting their proliferation. In some embodiments, upon internalization, the antibodies decrease the expression of immunosuppressive soluble factors while increasing expression of soluble factors that stimulate or promote the activity or proliferation of T cells, including CD4+ helper T cells and cytotoxic lymphocytes.

For certain therapeutic applications, the internalization process is employed for purposes of killing or decreasing the activity or proliferation of a target cell that expresses a LILRB2 protein. The number of antibody molecules internalized will be sufficient or adequate to kill a cell or inhibit its growth. Depending on the potency of an antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the targeted cell.

In some embodiments, the LILRB2 antibody or antigen-binding fragment provided herein is conjugated or linked to a therapeutic moiety, an imaging or detectable moiety, or an affinity tag. Methods for conjugating or linking polypeptides are well known in the art. Associations (binding) between compounds and labels include any means known in the art including, but not limited to, covalent and non-covalent interactions, chemical conjugation, as well as recombinant techniques. An antibody or antigen-binding fragment thereof is conjugated to, or recombinantly engineered with, an affinity tag (e.g., a purification tag), in some embodiments. Affinity tags such as, for example, poly-histidine (e.g., His6) tags are conventional in the art.

In some embodiments, the LILRB2 antibody or antigen-binding fragment further comprises a detectable moiety. Detections accomplished, for example, in vitro, in vivo or ex vivo. In vitro assays for the detection and/or determination (quantification, qualification, etc.) of, e.g., hLILRB2 protein expressed by macrophages using the antibodies or antigen-binding fragments thereof include but are not limited to, for example, ELISAs, RIAs, and western blots. In some embodiments, in vitro detection, diagnosis, or monitoring of the antigen of the antibodies occurs by obtaining a sample (e.g., a blood sample) from a subject and testing the sample in, for example, a standard ELISA assay.

### Derivatives

Derivatives of the LILRB2 antibody products described herein are also provided. The derivatized antibody product can comprise any molecule or substance that imparts a desired property to the antibody product, such as increased half-life in a particular use. The derivatized antibody product can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or streptavidin)), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). Examples of molecules that can be used to derivatize an antibody product include albumin (e.g., human serum albumin) and polyethylene glycol (PEG). Albumin-linked and PEGylated derivatives of antibody products can be prepared using techniques well known in the art. The antibody can be conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, polypropylene glycol homopolymers, polypropylene oxide/ethylene oxide co-polymers, polyoxyethylated polyols and polyvinyl alcohols.

Other derivatives include covalent or aggregative conjugates of LILRB2 antibody products, with other proteins or polypeptides, such as by expression of recombinant fusion proteins comprising heterologous polypeptides fused to the N-terminus or C-terminus of an LILRB2 antibody product. For example, the conjugated peptide can be a heterologous signal (or leader) polypeptide, e.g., the yeast alpha-factor leader, or a peptide such as an epitope tag. LILRB2 antibody product-containing fusion proteins can comprise peptides added to facilitate purification or identification of the LILRB2 antibody product (e.g., poly-His). An LILRB2 antibody product also can be linked to the FLAG peptide as described in Hopp et al., Bio/Technology 1988 6:1204-10, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

Oligomers that contain one or more LILRB2 antibody products can be employed as LILRB2 antagonists. Oligomers can be in the form of covalently-linked or non-covalently-linked dimers, trimers, or higher. Oligomers comprising two or more LILRB2 antibody products are contemplated for use, with one example being a homodimer. Other oligomers include heterodimers, homotrimers, heterotrimers, homotetramers, heterotetramers, etc.

Oligomers can comprise multiple LILRB2 antibody products joined via covalent or non-covalent interactions between peptide moieties fused to the LILRB2 antibody polypeptides. Such peptides can be peptide linkers (spacers), or peptides that have the property of promoting oligomerization. Leucine zippers and certain polypeptides derived from antibodies are among the peptides that can promote oligomerization of LILRB2 antibody products attached thereto, as described in more detail below.

Oligomers can comprise from two to four LILRB2 antibody products. The LILRB2 product moieties of the oligomer can be in any of the forms described above, e.g., variants or fragments. The oligomers comprise LILRB2 antibody products that have LILRB2 binding activity.

Preparation of fusion proteins comprising heterologous polypeptides fused to various portions of antibody-derived polypeptides (including the Fc domain) has been described, e.g., by Ashkenazi et al., Proc Natl Acad Sci USA. 1991 88(23):10535-9; Byrn et al., Nature 1990 344(6267):677-670; and Hollenbaugh and Aruffo, Curr Protoc Immunol. 2002 48(1):10.19A.1-10.19A.11.

Dimers are provided comprising two fusion proteins created by fusing a LILRB2 binding fragment of an LILRB2 antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One exemplary Fc polypeptide, described in PCT Publication No. WO 93/10151 and U.S. Pat. Nos. 5,426,048 and 5,262,522 (each of which is hereby incorporated by reference), is a single chain polypeptide extending from the N-terminal hinge region to the native C-terminus of the Fc region of a human IgG1 antibody. Another exemplary Fc polypeptide is the Fc mutein described in U.S. Pat. No. 5,457,035 and in Baum et al., EMBO J. 1994 13:3992-4001 (1994). The amino acid sequence of this mutein is identical to that of the native Fc sequence presented in PCT Publication No. WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

Alternatively, the oligomer is a fusion protein comprising multiple LILRB2 antibody polypeptides, with or without peptide linkers (spacer peptides). Among the suitable peptide linkers are those described in U.S. Pat. Nos. 4,751,180 and 4,935,233.

Another method for preparing oligomeric LILRB2 antibody product derivatives involves use of a leucine zipper. Leucine zipper domains are peptides that promote oligomerization of the proteins in which they are found. Examples of leucine zipper domains suitable for producing soluble oligomeric proteins are described in PCT Publication No. WO 94/10308, and the leucine zipper derived from lung surfactant protein D (SPD) described in Hoppe et al., FEBS Lett. 1994 344:191-5. The use of a modified leucine zipper that allows for stable trimerization of a heterologous protein fused thereto is described in Fanslow et al., Semin Immunol. 1994 6:267-78. Generally, recombinant fusion proteins comprising an LILRB2 antibody fragment fused to a leucine zipper peptide are expressed in suitable host cells, and the soluble oligomeric LILRB2 antibody products that form are recovered from the culture supernatant.

LILRB2 antibody products described herein can also be derivatized or modified such that the products have a longer half-life as compared to the underivatized or unmodified antibody. For example, the antibody product can contain point mutations to increase serum half-life, such as described in PCT Publication No. WO 00/09560.

### Nucleic Acids and Cells

Nucleic acids are provided that encode one or more chains of an antibody product herein, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing are also provided.

Nucleic acids provided encode antibody products disclosed herein, such as a light chain variable region shown in Table 10 or in Table 12, and/or a heavy chain variable region shown in Table 10 or in Table 12. Due to the degeneracy of the genetic code, each of the polypeptide sequences listed in Table 10 or in Table 12 is also encoded other nucleic acid sequences besides those listed in Table 10 or in Table 12. The present disclosure provides each degenerate nucleotide sequence encoding each antibody product.

The term “polynucleotide” or “nucleic acid” means single-stranded or double-stranded polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate, and phosphoroamidate. The term includes both single and double stranded forms.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences can include, in addition to the specified sequences, coding sequences for up to ten or even up to twenty other proteins or portions thereof, or can include operably linked regulatory sequences that control expression of the coding region of the recited nucleic acid sequences, and/or can include vector sequences.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “control sequence” refers to a polynucleotide sequence that can affect the expression and processing of coding sequences to which it is ligated. The nature of such control sequences can depend upon the host organism. For example, control sequences for eukaryotes can include promoters comprising one or a plurality of recognition sites for transcription factors, transcription enhancer sequences, and transcription termination sequence. “Control sequences” can include leader sequences and/or fusion partner sequences.

The term “vector” means any molecule or entity (e.g., nucleic acid, plasmid, bacteriophage, or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

DNA encoding antibody polypeptides (e.g., heavy or light chain, variable domain only, or full length) can be isolated from B-cells of mice that have been immunized with LILRB2 or an immunogenic fragment thereof. The DNA can be isolated by conventional procedures such as polymerase chain reaction (PCR). Phage display is another example of a known technique whereby nucleotide sequences encoding antibody polypeptides can be selected.

Nucleic acids are provided that hybridize to other nucleic acids under particular hybridization conditions. Methods for hybridizing nucleic acids are well-known in the art. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 96, 97, 98 or 99% identical to each other typically remain hybridized to each other.

The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., Chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA.

Changes can be introduced by mutation into a nucleic acid, thereby leading to changes in the amino acid sequence of a polypeptide (e.g., an antibody or antibody derivative) that it encodes. Mutations can be introduced using any technique known in the art. One or more particular amino acid residues can be changed using, for example, a site-directed mutagenesis protocol. One or more randomly selected residue can be changed using, for example, a random mutagenesis protocol. However it is made, a mutant polypeptide can be expressed and screened for a desired property.

Polypeptides that are components of an antibody product of interest are expressed in any suitable recombinant expression system.

Expression vectors are provided comprising nucleic acid encoding a LILRB2 antibody product. Examples of vectors include, but are not limited to, plasmids, viral vectors, non-episomal mammalian vectors and expression vectors, for example, recombinant expression vectors.

Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector can contain a “tag”-encoding sequence, that is, an oligonucleotide molecule located at the 5′ or 3′ end of the coding sequence, the oligonucleotide sequence encoding polyHis (such as hexaHis), or another “tag” for which commercially available antibodies exist, such as FLAG, HA (hemagglutinin from influenza virus), or myc. The tag is typically fused to the antibody protein upon expression, and can serve as a means for affinity purification of the antibody from the host cell. Affinity purification can be accomplished, for example, by column chromatography using antibodies against the tag as an affinity matrix. Optionally, the tag can subsequently be removed from the purified antibody polypeptide by various means such as using certain peptidases for cleavage.

Flanking sequences in the expression vector can be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence can be any prokaryotic or eukaryotic organism, any vertebrate or invertebrate organism, or any plant, provided that the flanking sequence is functional in, and can be activated by, the host cell machinery.

Expression and cloning vectors typically contain a promoter that is recognized by the host organism and operably linked to nucleic acid encoding the LILRB2 antibody product. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continuous gene product production; that is, there is little or no experimental control over gene expression. A large number of promoters, recognized by a variety of potential host cells, are well known. A suitable promoter is operably linked to the DNA encoding LILRB2 antibody product by removing the promoter from the source DNA by restriction enzyme digestion or amplifying the promoter by polymerase chain reaction and inserting the desired promoter sequence into the vector.

Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and—most preferably Simian Virus 40 (SV40). Other suitable mammalian promoters include heterologous mammalian promoters, for example, heat-shock promoters and the actin promoter.

Exemplary promoters useful in recombinant expression vectors include, but are not limited to: the SV40 early promoter region (Bemoist and Chambon, 1981, Nature, 290:304-10); the CMV promoter; the promoter contained in the 3′ long terminal repeat of Rous sarcoma virus (Yamamoto, et al., Cell 1980 22:787-97); the herpes thymidine kinase promoter (Wagner et al., Proc Natl Acad Sci USA. 1981 78:1444-5); the regulatory sequences of the metallothionine gene (Brinster et al., Nature. 1982 296:39-42); prokaryotic expression vectors such as the beta-lactamase promoter (Villa-Komaroff et al., Proc Natl Acad Sci USA 1978 75:3727-31); or the tac promoter (DeBoer et al., Proc Natl Acad Sci USA. 1983 80:21-5). Also available for use are the following animal transcriptional control regions, which exhibit tissue specificity and have been utilized in transgenic animals: the elastase I gene control region that is active in pancreatic acinar cells (Swift et al., Cell. 1984 38:639-46; Ornitz et al., Cold Spring Harb Symp Quant Biol. 1986 50:399-409; MacDonald, Hepatology. 1987 7:425-515); the insulin gene control region that is active in pancreatic beta cells (Hanahan, Nature. 1985 315:115-22); the mouse mammary tumor virus control region that is active in testicular, breast, lymphoid and mast cells (Leder et al., Cell 1986 45:485-95); the albumin gene control region that is active in liver (Pinkert et al., Genes Dev 1987 1:268-76); the alpha-feto-protein gene control region that is active in liver (Krumlauf et al., Mol Cell Biol 1985 5:1639-48; Hammer et al., Science. 1987 235:53-8); the alpha 1-antitrypsin gene control region that is active in the liver (Kelsey et al., Genes Dev. 1987 1:161-71); the beta-globin gene control region that is active in myeloid cells (Mogram et al., Nature. 1985 315:338-40; Kollias et al., Cell 1986 46:89-94); the myelin basic protein gene control region that is active in oligodendrocyte cells in the brain (Readhead et al., Cell. 1987 48:703-12); the myosin light chain-2 gene control region that is active in skeletal muscle (Sani, Nature. 1985 314:283-6); the gonadotropic releasing hormone gene control region that is active in the hypothalamus (Mason et al., Science. 1986 234:1372-8); and most particularly the immunoglobulin gene control region that is active in lymphoid cells (Grosschedl et al., Cell 1984 38:647-58; Adams et al., Nature. 1985 318:533-8; Alexander et al., Mol Cell Biol. 1987 7:1436-44).

An enhancer sequence can be inserted into the vector to increase the transcription in higher eukaryotes of a nucleic acid encoding an LILRB2 antibody product described herein. Various enhancer sequences available from mammalian genes are known (e.g., globin, elastase, albumin, alpha-feto-protein and insulin). An enhancer sequence from a virus also can be used. The SV40 enhancer, the cytomegalovirus early promoter enhancer, the polyoma enhancer, and adenovirus enhancers are exemplary enhancing elements for the activation of eukaryotic promoters. While an enhancer can be spliced into the vector at a position 5′ or 3′ to a nucleic acid molecule, it is typically placed at a site 5′ to the promoter.

In expression vectors, a transcription termination sequence is typically located 3′ of the end of a polypeptide-coding region and serves to terminate transcription. A transcription termination sequence used for expression in prokaryotic cells typically is a G-C rich fragment followed by a poly-T sequence. While the sequence is easily cloned from a library or even purchased commercially as part of a vector, it can also be readily synthesized using methods for nucleic acid synthesis such as those described herein.

Selection genes can be used to amplify the gene that will be expressed. Amplification is a process whereby genes that cannot in single copy be expressed at high enough levels to permit survival and growth of cells under certain selection conditions are reiterated in tandem within the chromosomes of successive generations of recombinant cells. Examples of suitable amplifiable selectable markers for mammalian cells include dihydrofolate reductase (DHFR) and promoterless thymidine kinase. In the use of these markers mammalian cell transformants are placed under selection pressure in which only the transformants are uniquely adapted to survive by virtue of the selection gene present in the vector. Selection pressure is imposed by culturing the transformed cells under conditions in which the concentration of selection agent in the medium is successively increased, thereby permitting survival of only those cells in which the selection gene has been amplified. Under these circumstances, DNA adjacent to the selection gene, such as DNA encoding an antibody, is co-amplified with the selection gene. As a result, increased quantities of LILRB2 polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, for example where glycosylation is desired in a eukaryotic host cell expression system, various presequences can be manipulated to improve glycosylation or yield. For example, the peptidase cleavage site of a particular signal peptide can be altered, or pro-sequences added, which also can affect glycosylation. The final protein product can have, in the −1 position (relative to the first amino acid of the mature protein) one or more additional amino acids incident to expression, which may not have been totally removed. For example, the final protein product can have one or two amino acid residues found in the peptidase cleavage site, attached to the amino terminus. Alternatively, use of some enzyme cleavage sites can result in a slightly truncated yet active form of the desired polypeptide, if the enzyme cuts at such area within the mature polypeptide.

Where a commercially available expression vector lacks some of the desired flanking sequences as described above, the vector can be modified by individually ligating these sequences into the vector. After the vector has been chosen and modified as desired, a nucleic acid molecule encoding an LILRB2 antibody product is inserted into the proper site of the vector.

The completed vector containing sequences encoding the antibody product is inserted into a suitable host cell for amplification and/or polypeptide expression. The transformation of an expression vector for an LILRB2 antibody product into a selected host cell can be accomplished by well-known methods including methods such as transfection, infection, calcium chloride, electroporation, microinjection, lipofection, DEAE-dextran method, or other known techniques. The method selected will in part be a function of the type of host cell to be used. These methods and other suitable methods are well known to the skilled artisan.

Antibodies can be expressed in hybridoma cell lines or in cell lines other than hybridomas. Expression constructs encoding the antibodies can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216, 4,912,040, 4,740,461, and 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

The transformed host cell, when cultured under appropriate conditions, synthesizes an LILRB2 antibody product that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (ATCC), such as Chinese hamster ovary (CHO) cells, HeLa cells, baby hamster kidney (BHK) cells, monkey kidney cells (COS), human hepatocellular carcinoma cells (e.g., Hep G2), human embryonic kidney cells (HEK) (e.g., HEK293) and a number of other cell lines. The best cell line for expressing a particular DNA construct can be selected by testing various cell lines to determine which ones have the highest levels of expression levels and produce antibody products with the desired LILRB2 binding properties.

Nucleic acid molecules that are suitable for use as primers or hybridization probes for the detection of nucleic acid sequences are also provided. A nucleic acid molecule can comprise only a portion of a nucleic acid sequence encoding a full-length polypeptide, for example, a fragment that can be used as a probe or primer or a fragment encoding an active portion (e.g., a LILRB2 binding portion) of a polypeptide.

### Compositions

Compositions that include LILRB2 antibody products are also provided.

Pharmaceutical compositions typically include one or more of a buffer, a pharmaceutically acceptable diluent, a carrier, a solubilizer, an emulsifier and a preservative. The use of the foregoing antibody products in the preparation of a pharmaceutical composition or medicament is also provided.

Acceptable formulation components for pharmaceutical preparations are nontoxic to recipients at the dosages and concentrations employed. In addition to the antibody products that are provided herein, compositions can contain components for modifying, maintaining or preserving, for example, the pH, osmolarity, viscosity, clarity, color, isotonicity, odor, sterility, stability, rate of dissolution or release, adsorption or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remingtons Pharmaceutical Sciences, 23rd ed., (Adejare, ed.), 2020, Elsevier Academic Press).

The primary vehicle or carrier in a pharmaceutical composition can be either aqueous or non-aqueous in nature. Suitable vehicles or carriers for such compositions include water for injection, physiological saline solution or artificial cerebrospinal fluid, possibly supplemented with other materials common in compositions for parenteral administration. Neutral buffered saline or saline mixed with serum albumin are further exemplary vehicles. Compositions comprising LILRB2 antibody product can be prepared for storage by mixing the selected composition having the desired degree of purity with optional formulation agents in the form of a lyophilized cake or an aqueous solution. Further, the LILRB2 antibody product can be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which can further include sorbitol or a suitable substitute therefor.

Additional pharmaceutical compositions are in the form of sustained- or controlled-delivery formulations. Techniques for formulating a variety of other sustained- or controlled-delivery means, such as liposome carriers, bio-erodible microparticles or porous beads and depot injections can be used (see, e.g., PCT Publication No. WO 93/15722 A1, which describes the controlled release of porous polymeric microparticles for the delivery of pharmaceutical compositions). Sustained-release preparations can include semipermeable polymer matrices in the form of shaped articles, e.g., films, or microcapsules, polyesters, hydrogels, polylactides (U.S. Pat. No. 3,773,919 and EP 058,481), copolymers of L-glutamic acid and gamma ethyl-L-glutamate (Sidman et al., Biopolymers 1983 22:547-56), poly (2-hydroxyethyl-methacrylate) (Langer et al., J Biomed Mater Res. 1981 15:167-277) and Langer, Chem Tech. 1982 12:98-105), ethylene vinyl acetate (Langer et al., ibid.) or poly-D(−)-3-hydroxybutyric acid (EP 133,988). Sustained release compositions can also include liposomes, which can be prepared by any of several methods known in the art. See, e.g., Eppstein et al., Proc Natl Acad Sci USA. 1985 82:3688-92; EPO Publication Nos. EP 036676; EP 088046, and EP 143949.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) that is reconstituted prior to administration.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Kits are provided for multi-dose or single-dose administration units. For example, kits can each contain both a first container having a dried protein and a second container having an aqueous diluent, including for example single and multi-chambered pre-filled syringes (e.g., liquid syringes, lyosyringes or needle-free syringes).

The pharmaceutical compositions can be delivered parenterally, typically by injection. Injections can be intraocular, intraperitoneal, intraportal, intramuscular, intravenous, intrathecal, intracerebral (intra-parenchymal), intracerebroventricular, intraarterial, intralesional, perilesional, or subcutaneous. Eye drops can be used for intraocular administration. In some instances, injections can be localized to the vicinity of a particular bone or bones to which the treatment is targeted. For parenteral administration, the antibodies can be administered in a pyrogen-free, parenterally acceptable aqueous solution comprising the desired LILRB2 antibody product in a pharmaceutically acceptable vehicle. A particularly suitable vehicle for parenteral injection is sterile distilled water in which the LILRB2 antibody product are formulated as a sterile, isotonic solution, properly preserved.

Pharmaceutical compositions comprising the subject LILRB2 antibody products can be administered by bolus injection or continuously by infusion, by implantation device, sustained release systems or other means for accomplishing prolonged release. The pharmaceutical composition also can be administered locally via implantation of a membrane, sponge or another appropriate material onto which the desired molecule has been absorbed or encapsulated. Where an implantation device is used, the device can be implanted into any suitable tissue or organ, and delivery of the desired molecule can be via diffusion, timed-release bolus, or continuous release. The preparation can be formulated with agents, such as injectable microspheres, bio-erodible particles, polymeric compounds (such as polylactic acid; polyglycolic acid; or copoly (lactic/glycolic) acid (PLGA), beads or liposomes, that can provide controlled or sustained release of the product which can then be delivered via a depot injection. Formulation with hyaluronic acid has the effect of promoting sustained duration in the circulation.

Subject compositions comprising LILRB2 antibody product also can be used ex vivo. In such instances, cells, tissues, or organs that have been removed from the patient are exposed to or cultured with the LILRB2 antibody product. The cultured cells can then be implanted back into the patient or a different patient or used for other purposes.

LILRB2 antibody product can be delivered by implanting certain cells that have been genetically engineered, using methods such as those described herein, to express and secrete the polypeptide. Such cells can be animal or human cells, and can be autologous, heterologous, or xenogeneic, or can be immortalized. In order to decrease the chance of an immunological response, the cells can be encapsulated to avoid infiltration of surrounding tissues. Encapsulation materials are typically biocompatible, semi-permeable polymeric enclosures or membranes that allow the release of the protein product(s) but prevent the destruction of the cells by the patient's immune system or by other detrimental factors from the surrounding tissues.

As used herein, “substantially pure” means that the described species of molecule is the predominant species present, that is, on a molar basis it is more abundant than any other individual species in the same mixture. A substantially pure molecule can be a composition in which the object species comprises at least 50% (on a molar basis) of all macromolecular species present. A substantially pure composition can comprise at least 80%, 85%, 90%, 95%, or 99% of all macromolecular species present in the composition. The object species can also be purified to essential homogeneity in which contaminating species cannot be detected in the composition by conventional detection methods and thus the composition consists of a single detectable macromolecular species.

### Dosages

The pharmaceutical compositions that are provided can be administered for prophylactic and/or therapeutic treatment.

As used herein, the terms “treatment,” “treating,” and the like, refer to administering an agent or carrying out a procedure, for the purposes of obtaining an effect. The effect is prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder (e.g., cancer) in a mammal, particularly in a human, and includes: (a) preventing the disease or a symptom of a disease from occurring in a subject which is predisposed to the disease but has not yet been diagnosed as having it (e.g., including diseases that are associated with or caused by a primary disease); (b) inhibiting the disease, i.e., arresting its development; and (c) relieving the disease, i.e., causing regression of the disease. Treating refers to any clinical indicia of success in the treatment or amelioration or prevention, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present disclosure to prevent or delay, to alleviate, or to arrest or inhibit development of the symptoms or conditions associated with diseases (e.g., cancer). The term “therapeutic effect” refers to the reduction, elimination, or prevention of the disease, symptoms of the disease, or side effects of the disease in the subject. For example, a subject is “treated” for a disease or disorder if, after receiving a therapeutic amount of a combination of a LILRB2 antibody product provided herein, the patient shows one or more observable and/or measurable changes in an endpoint or symptom of the disease condition.

An “effective response” in accordance with the present disclosure is achieved when the subject experiences partial or total alleviation or reduction of signs or symptoms of illness and, in the case of the treatment of cancer, specifically includes, without limitation, amelioration of symptoms, retarding progression, cure, remission, prolongation of survival, or other objective responses. The expected progression-free survival times can be measured in months to years, depending on prognostic factors including the number of relapses, stage of disease, and other factors. Prolonging survival includes without limitation times of at least 1 month (mo.), about at least 2 mos., about at least 3 mos., about at least 4 mos., about at least 6 mos., about at least 1 year, about at least 2 years, about at least 3 years, etc. Overall survival is also measured, for example, in months to years. Alternatively, an effective response can be that a subject's symptoms remain static.

Administration of a therapeutic agent in a prophylactic method occurs prior to the manifestation of symptoms of an undesired disease or disorder, such that the disease or disorder is prevented or, alternatively, delayed in its progression. Thus, when used in conjunction with prophylactic methods, the term “therapeutically effective” means that, after treatment, a smaller number of subjects (on average) develop the undesired disease or disorder or progress in severity of symptoms

The terms “recipient,” “individual,” “subject,” “host,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. The mammal can be a human.

In general, toxicity and therapeutic efficacy of the antibody product can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The effective amount of a pharmaceutical composition comprising LILRB2 antibody product to be employed therapeutically or prophylactically will depend, for example, upon the therapeutic context and objectives. One skilled in the art will appreciate that the appropriate dosage levels for treatment, will thus vary depending, in part, upon the molecule delivered, the indication for which the LILRB2 antibody is being used, the route of administration, and the size (body weight, body surface or organ size) and/or condition (the age and general health) of the patient. A clinician can titer the dosage and modify the route of administration to obtain the optimal therapeutic effect. Typical dosages range from about 1 mg/kg to up to about 1600 mg/kg or more, depending on the factors mentioned above. The dosage can range from 1 mg/kg up to about 200 mg/kg; or 1 mg/kg up to about 1200 mg/kg; or 1 ug/kg up to about 1600 mg/kg.

The dosing frequency will depend upon the pharmacokinetic parameters of the LILRB2 antibody product in the formulation. For example, a clinician will administer the composition until a dosage is reached that achieves the desired effect. The composition can therefore be administered as a single dose, or as two or more doses (which can contain the same amount of the desired molecule) over time, or as a continuous infusion via an implantation device or catheter. Treatment can be continuous over time or intermittent. Further refinement of the appropriate dosage is routinely made by those of ordinary skill in the art and is within the ambit of tasks routinely performed by them. Appropriate dosages can be ascertained through use of appropriate dose-response data. An exemplary dosing schedule is every 2 to 3 weeks.

To treat a disease condition by targeting LILRB2, a composition comprising the subject LILRB2 antibody product is administered to the patient in an amount and for a time sufficient to induce a sustained improvement in at least one indicator that reflects the severity of the disorder. An improvement is considered “sustained” if the patient exhibits the improvement on at least two occasions separated by at least one to seven days, or in some instances one to six weeks. The appropriate interval will depend to some extent on what disease condition is being treated; it is within the purview of the skilled physician to determine the appropriate interval for determining whether the improvement is sustained. The degree of improvement is determined based on signs or symptoms, and can also employ questionnaires that are administered to the patient, such as quality-of-life questionnaires.

Various indicators that reflect the extent of the patient's illness can be assessed for determining whether the amount and time of the treatment is sufficient. The baseline value for the chosen indicator or indicators is established by examination of the patient prior to administration of the first dose of antibody. Preferably, the baseline examination is done within about 60 days of administering the first dose. If the antibody is being administered to treat acute symptoms, such as for example to treat a broken bone, the first dose is administered as soon as practically possible after the injury has occurred.

Improvement is induced by administering the subject LILRB2 antibody product until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions. For injuries or acute conditions, a single dose can be sufficient.

Although the extent of the patient's illness after treatment may appear improved according to one or more indicators, treatment can be continued indefinitely at the same level or at a reduced dose or frequency. Once treatment has been reduced or discontinued, it later can be resumed at the original level if symptoms should reappear.

### Methods of Use

The LILRB2 antibody products disclosed herein have a variety of utilities. Some of the antibodies and fragments, for instance, are useful for specific binding assays, affinity purification of LILRB2 or its ligands, and screening assays to identify other antagonists of LILRB2 activity. The antibody products can be used to treat various diseases that are associated with the activity of LILRB2.

LILRB2 antibody products can be used to detect LILRB2 in biological samples. Such uses allow the identification of cells or tissues that produce the protein or serve as a diagnostic for detecting pathological conditions in which LILRB2 is overproduced or underproduced. The strong expression of LILRB2 by macrophages, osteoclasts, and other myeloid cells can be a marker of the activity of those cells, and detection of LILRB2 expression on myeloid cells can be used as a marker of a disease or disorder characterized by the cell type in question. Likewise, detection of LILRB2 expression by cancer cells can be used to identify subjects whose cancer may be amenable to treatment with therapeutic LILRB2 antibody product methods disclosed herein.

Accordingly, methods are provided of detecting cell activity in a biological sample, such as a sample of an in vitro medium or a tissue sample from a subject, or in vivo in a subject, in which the method comprises contacting the cell expressing LILRB2 with an LILRB2 antibody product provided herein. The LILRB2 antibody product can be conjugated to a detectable moiety, and the method comprises detecting the moiety directly. The method can comprise detecting binding of the LILRB2 antibody product to the cell indirectly by way of a detectably moiety that binds to the antibody. For example, an IgG antibody conjugated to a detectable moiety can be used to bind to the LILRB2 antibody presented as an IgG isotype. The cell can be a tumor cell. The cell can be a myeloid cell (e.g., a monocyte, dendritic cell, macrophage, myeloid-derived suppressor cell, tumor-associated macrophage, immunosuppressive macrophage or M2-like macrophage) or an osteoclast.

The antibody products provided can also be used in methods to screen for a molecule that binds to LILRB2. A variety of competitive screening methods, for example, can be used. In some methods, a LILRB2 molecule or fragment thereof to which an LILRB2 antibody product binds, is contacted with an antibody product disclosed herein together with another molecule (i.e., a candidate molecule). A reduction in binding between the antibody product and LILRB2 is an indication that the candidate molecule binds LILRB2. Binding of the antibody product can be detected using a variety of methods, e.g., an ELISA. Detection of binding between the LILRB2 antibody product and LILRB2 can be simplified by detectably labeling the antibody. In some methods, a molecule that exhibits binding in the initial screen is further analyzed to determine whether it inhibits or modulates a LILRB2 activity.

The LILRB2 antibody products provided herein are useful for the treatment of human disease, including cancers.

LILRB2 antibody products described herein can be used for the treatment of cancer, either alone or in combination with another anti-cancer therapeutic. Cancers to be treated are those where the cancer cells are known to express LILRB2 or are of a type that has previously been observed to express LILRB2. Certain cancers that are EGFR-mutant have been found to more highly express LILRB2, and so are contemplated for treatment using the antibodies disclosed herein. In contrast, expression of LILRB2 in cancer cells has also been found to be inversely related to expression of PD-L1. Anti-LILRB2 treatment as described herein is thus also contemplated in cases where PD-L1 expression by the cancer cells is not observed. Such treatment is indicated when therapeutic intervention in the PD-1/PD-L1 axis is, or is expected to be, ineffective.

Provided herein are methods of treating a patient having a cancer, in which a LILRB2 antibody product mediates killing of the cells of the cancer.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers.

Examples of cancers that can be treated by methods and compositions provided herein include, but are not limited to, cancers of the bladder, blood, bone, bone marrow, brain, breast, cervix, colon, esophagus, gastrointestinal tract, rectum, head and neck, kidney, larynx, liver, lung, mouth nasopharynx, neck, ovary, pancreas, prostate, skin, stomach, testis, thyroid, tongue, and uterus.

Cancers to be treated include, for example, acute lymphoblastic leukemia, chronic lymphocytic leukemia, acute myeloid leukemia, myelodysplastic syndrome, chronic myelogenous leukemia, Hodgkin's disease; Hodgkin's lymphoma, non-Hodgkin's lymphoma, Burkitt lymphoma, bladder cancer, breast cancer, cervical cancer, colorectal cancer, endometrial cancer, esophageal cancer, gallbladder cancer, hepatocellular cancer, head and neck cancer, kidney cancer, melanoma, malignant mesothelioma, nasopharyngeal cancer, neuroblastoma, glioblastoma, pancreatic cancer, multiple myeloma, prostate cancer, small cell lung cancer, non-small cell lung cancer, and metastatic cancers. Cancers to be treated include, for example, glioblastoma multiforme, head and neck cancer, kidney renal clear cell cancer, acute myeloid leukemia, pancreatic adenocarcinoma, skin cutaneous melanoma, stomach adenocarcinoma, testicular germ cell cancer, gastric cancer, Merkel cell carcinoma, dendritic sarcoma, non-small cell lung cancer, papillary thyroid cancer, cutaneous squamous cell carcinoma, or ovarian cancer.

In addition, the cancer can specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial adenomatous polyposis; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; malignant cellular blue nevus; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides or cutaneous T-cell lymphoma; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As an example, a method is provided for the treatment of a subject suffering from melanoma. As used herein, “melanoma” refers to a condition characterized by the growth of a tumor arising from the melanocytic system of the skin and other organs. Most melanocytes occur in the skin, but are also found in the meninges, digestive tract, lymph nodes and eyes. When melanoma occurs in the skin, it is referred to as cutaneous melanoma. Melanoma can also occur in the eyes and is called ocular or intraocular melanoma. Melanoma occurs rarely in the meninges, the digestive tract, lymph nodes or other areas where melanocytes are found.

Cells of a cancer treated in methods provided herein can express LILRB2. Cells of the cancer can overexpress LILRB2.

A way by which a LILRB2 antibody product can mediate cancer cell killing is through antibody-dependent cellular toxicity (“ADCC”). ADCC is the process by which antibodies coat a target cell (e.g., a cancer cell or bacterial cell) and recruit effector cells to induce target cell death via non-phagocytic mechanisms.

As noted above, in treatment methods the LILRB2 antibody products provided herein can be used as a monotherapy or in a combination therapy. A “combination” therapy refers to administration of one treatment agent before, during, or after administration of the other treatment agent to the subject.

In a combination treatment for cancer, the LILRB2 antibody product is used in combination with one or more other anticancer modalities for the treatment of a cancer in a subject. The anticancer modality can be a chemotherapeutic or biologic molecule. The anticancer modality can be an immunotherapeutic molecule. The immunotherapeutic can be a checkpoint inhibitor. The checkpoint inhibitor can be a PD-1 antagonist. The checkpoint inhibitor can be a PD-L1 antagonist.

Provided herein are methods of treating a patient having a cancer, comprising administering to the patient an LILRB2 antibody product as described herein in combination with an immunotherapy.

As an example of a combination with an immunotherapy, methods of treating a patient having a cancer are provided, comprising administering to the patient a therapeutically effective amount of a LILRB2 antibody product and one or more immune checkpoint inhibitors.

The term “immune checkpoint inhibitor” or “checkpoint inhibitor” generally refers to an agent that modulates an immune checkpoint protein (a “checkpoint protein”). A checkpoint inhibitor can achieve total or partial reduction, inhibition, interference to an activity of the checkpoint protein, or produce other changes to structure of the checkpoint protein that change binding of a checkpoint protein to a ligand, and/or impact a pathway related to activity of the checkpoint protein such as, for example, by acting as an antagonist to a checkpoint protein or a ligand of the checkpoint protein. The immune checkpoint inhibitor can be a compound such as an antibody or other protein that binds and antagonizes human programmed cell death protein 1 (PD-1; aka PDCD1, CD279) or programmed cell death ligand 1 (PD-L1; aka BZ-H1, CD274). Such immune checkpoint inhibitors are termed, respectively, PD-1 antagonists and PD-L1 antagonists.

As disclosed herein, checkpoint proteins can, in certain contexts and states, interfere with T-cell mediated killing of cancer cells. Checkpoint inhibitors can reverse the interference of checkpoint proteins but interference with a checkpoint protein is not enough in certain types of cancers (e.g., certain solid tumors). The present disclosure contemplates that combining an antibody product provided herein with another a checkpoint inhibitor relieves macrophage-mediated T-cell exhaustion and stimulates T-cell effector function.

A checkpoint inhibitor can inhibit one or more checkpoint proteins. Non-limiting examples of checkpoint proteins include: PD-1, CD28, CTLA-4, ICOS, TMIGD2, 4-1BB, BTLA, CD160, LIGHT, LAG3, OX40, CD27, CD40L, CD47, GITR, DNAM-1, TIGIT, CD96, PVRIG 2B4, TIM-3, Galectin9, CEACAMi, SIRP alpha, DC-SIGN, CD200R, DR3, CDCHK1, CHK2, A2aR, or B-7 family proteins.

A checkpoint inhibitor can interact with a ligand of a checkpoint protein. For instance, by way of non-limiting example, a ligand of a checkpoint protein includes: PD-L1, PD-L2, ICOS ligand, VISTA, 4-1BBL, Herpesvirus Entry Mediator (HVEM), tumor necrosis factor receptor superfamily member 14 or TNFRSF14, MHC class I, MHC class II, PVR, OX-40L, CD70, CD40, GITRL, CD155, CD48, GAL9; HMGB1, CEASAM-1, phosphatidyl serine (PtdSer), IDO, TDO, CD47, BTN2A1, CD200, TL1A, CD112, CD155, MHCII, LSECtin, CHK1, CHK2, A2aR, or a B-7 family ligand (e.g., CD80 (B7-1), CD86 (B7-2), B7-H3, B7-H4, B7-H7 (HHLA2), etc.).

A checkpoint inhibitor can be an antagonist. For example, a checkpoint inhibitor can antagonize a checkpoint protein. A checkpoint inhibitor can be an antagonist to a ligand of a checkpoint protein. The antagonist can be a biological molecule such as a biological therapeutic. A checkpoint inhibitor can be an antibody or an antigen-binding portion thereof, such as a monoclonal antibody, a humanized antibody, a fully human antibody, a fusion protein, or a combination thereof. A checkpoint inhibitor can be a small molecule. A checkpoint inhibitor can be a rationally-designed peptide. A checkpoint inhibitor can be a cell or cell preparation (e.g., cells that express a checkpoint inhibitor).

The checkpoint inhibitor can inhibit PD-1. Programmed cell death 1 (PD-1) is a key checkpoint receptor expressed by activated T and B cells, and mediates immunosuppression. Among other things, PD-1 limits activity of T cells in peripheral tissues during an inflammatory response to infection. In addition, as a checkpoint protein, PD-1 blockade can enhance T-cell proliferation and cytokine production in response to a challenge by specific antigen targets or by allogeneic cells in mixed lymphocyte reactions.

Without intending to be bound by theory, it is believed that blockade of PD-1, in combination with a LILRB2 antibody product as disclosed herein, relieves macrophage-mediated T cell suppression/exhaustion, increases T cell proliferation and cytokine production, and improves immune cell effector function. PD-1 blockade can be accomplished by a variety of mechanisms. For instance, PD-1 blockade can be achieved by blocking PD-1 binding to its ligands. PD-1 can be blocked with a checkpoint inhibitor that is a PD-1 antagonist. For instance, a PD-1 antagonist can be a PD-1 antibody (e.g., nivolumab, pembrolizumab, etc.). A PD-1 antagonist can be a small molecule [e.g., INCB-086550 (Incyte) or small molecules disclosed in, e.g., Wu et al., Acta Pharmacol Sin. 2021 42:1-9; Jiao et al., Curr Pharm Des. 2018 24(41):4911-20; and Liu et al., Cancer Cell Int. 2021 21(1):239]. A PD-1 antagonist can be or can comprise rationally-designed peptide (e.g., APi2568). A PD-1 antagonist can be or can comprise a cell or cell preparation (e.g., cells that express a PD-1 binding agent, e.g., a PD-1 antibody, e.g., HerinCAR-PD1).

Exemplary PD-1 antibodies suitable for use in the methods include, without limitation, nivolumab (ONO-4538, BMS-936558, MDX1106, Opdivo®; Bristol-Myers Squibb), pembrolizumab (MK-3475, Keytruda®; Merck), cemipilimab (e.g., cemiplimab-rwlc (Libtayo™; Regeneron)), dostarlimab (e.g., dostarlimab-gxly (Jemperli™; GlaxoSmithKline)), pimivalimab (IgG4)(JTX-4014; Jounce Therapeutics), spartalizumab (IgG4)(PDR001; Novartis), camrelizumab (SHR1210; Jiangsu HengRui Medicine), sintilimab (IBI308; Innovent and Eli Lilly), tislelizumab (BGBA317; BeiGene), toripalimab (JS 001; Shanghai Junshi Bioscience), INCMGA00012 (MGA012; Incyte and MacroGenics), AMP-224 (PD-L2/Ig fusion; AstraZeneca/MedImmune and GlaxoSmithKline), AMP-514 (IgG4K) (MEDI0680; AstraZeneca), balstilimab (AGEN2034; Agenus), and/or a PD-1 binding domain of any of them.

Another exemplary PD-1 antagonist is a rationally designed peptide such as, e.g., APi2568, which comprises a B-cell epitope (amino acids 92-110 from PD-1) linked to a promiscuous T-cell epitope (amino acid residues 288-302 from measles virus fusion protein) via a 4-amino acid linker, and combined with Water for Injection (WFI) forms the drug product, IMU-201, which becomes PD1-Vaxx when emulsified with excipient Montanide ISA 720 VG.

Another exemplary PD-1 antagonist is a cell expressing a PD-1 antibody, for example, PD-1 antibody-expressing-CAR-T cells (e.g., HerinCAR-PD1 cells).

In some embodiments, the checkpoint inhibitor is a PD-L1 antagonist, such as a PD-L1 antibody. In some embodiments, the PD-L1 antibody is selected from the group consisting of avelumab, durvalumab, atezolizumab, envafolimab, cosibelimab, LY3300054 CA-170, BMS-936559, and PD-L1-binding fragments or combinations thereof. In some embodiments, the PD-L1 antagonist comprises AUNP-12, BMS-986189, a PD-L1 binding domain comprising CDRs of an antibody selected from the group consisting of avelumab, durvalumab, atezolizumab, envafolimab, cosibelimab (CK-301), LY3300054, CA-170, and BMS-936559, and active fragments thereof, or combinations thereof.

A checkpoint inhibitor can inhibit cytotoxic T-lymphocyte-associated protein 4 (CTLA-4) or a ligand thereof. CTLA-4 antibodies bind to CTLA-4 and block the interaction of CTLA-4 with its ligands CD80/CD86, which ligands are expressed on antigen presenting cells. Accordingly, a CTLA-4 inhibitor that blocks interaction of CTLA-4 and its ligands can block negative down regulation of the immune responses elicited by the interaction of these molecules. Thus, a checkpoint inhibitor can be a CTLA-4 antagonist such as described in U.S. Pat. Nos. 5,811,097; 5,811,097; 5,855,887; 6,051,227; 6,207,157; 6,682,736; 6,984,720; and 7,605,238. Moreover, exemplary CTLA-4 antibodies include: ipilimumab (10D1, MDX-D010, Yervoy™; Bristol-Myers Squibb), tremelimumab (ticilimumab, CP-675,206; MedImmune), and quavonlimab (MK-1308; Merck). A CTLA-4 antagonist can comprise a CTLA-4-binding domain or fragment thereof of any CTLA-4 antagonist. A CTLA-4 antagonist can comprise a small molecule (see, e.g., Wang et al., Biochim Biophys Acta Rev Cancer. 2019 1871(2):199-224).

Lymphocyte-activation gene 3 (LAG-3, also known as CD223) is a CD4− related transmembrane protein that competitively binds MHC II and acts as a co-inhibitory checkpoint for T cell activation [e.g., Goldberg and Drake, Curr Top Microbiol Immunol. 2011 344:269-78]. A checkpoint inhibitor can be a LAG3 antagonist. A LAG3 antagonist can be a LAG-3-binding protein (e.g., an antibody) or a protein that binds to a LAG3 ligand. Non-limiting examples of LAG-3 antibodies include: LAG525 (IMP701, Novartis/Prima Biomed), MK-4280 (Merck Sharp & Dohme), REGN3767 (Regeneron Pharmaceuticals), relatlimab (BMS-986016, Bristol-Myers Squibb), and BI 754111 (Boehringer Ingelheim).

T cell immunoglobulin mucin 3 (TIM-3, also known as Hepatitis A virus cellular receptor (HAVCR2)) is a type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Binding of Gal-9 by the TIM-3 receptor triggers downstream signaling to negatively regulate T cell survival and function. A checkpoint inhibitor can be an agent that inhibits TIM-3. A checkpoint inhibitor can be a TIM-3 antagonist, such as a TIM3 antibody or an antibody to a TIM-3 ligand. A TIM-3 antagonist can comprise a TIM-3-binding domain or fragment thereof of any TIM-3 antagonist. Non-limiting examples of TIM-3 antagonists include: TSR-022 (AnaptysBio/Tesaro, Inc.) and MGB453 (Novartis). Additional exemplary TIM-3 binding proteins (e.g., antibodies) are known in the art and are disclosed, e.g., in U.S. Pat. Nos. 9,103,832, 8,552,156, 8,647,623, 8,841,418; U.S. Patent Publication Nos. 2016/0200815, 2015/0284468, 2014/0134639, 2014/0044728, 2012/0189617, 2015/0086574, 2013/0022623; and PCT Publication Nos. WO2016/068802, WO2016/068803, WO2016/071448, WO2011/155607, and WO2013/006490.

T cell immunoglobulin and ITIM domain (TIGIT) is an inhibitory receptor expressed on lymphocytes. TIGIT interacts with CD155 expressed on antigen-presenting cells or tumor cells to down-regulate T cell and natural killer (NK) cell functions. A checkpoint inhibitor can be a TIGIT antagonist. A TIGIT antagonist cam bins to TIGIT or to a TIGIT ligand. A TIGIT antagonist can be a TIGIT antibody or an antibody to a TIGIT ligand. A TIGIT antagonist comprises a TIGIT-binding domain or fragment thereof of any TIGIT antagonist. Non-limiting examples of TIGIT antagonists include: Tiragolumab (MTIG7192A; RG6058) (Genentech/Roche), AB154 (Arcus Bioscience), vibostolimab (MK-7684)(Merck), BMS-985207 (Bristol-Myers Squibb), ASP8374 (Astellas Pharma; Potenza Therapeutics), and ASP8374 (Astellas Pharma; Potenza Therapeutics).

Exemplary anti-CD27 agonists include MK-5890 (Merck).

Exemplary ICOS antibodies include vopratelimab (JTX-2011; Jounce).

In some embodiments, the LILRB2 antibody product and an immune checkpoint inhibitor are co-formulated. In some embodiments, the LILRB2 antibody product and the immune checkpoint inhibitor are in separate formulations. In some embodiments, the LILRB2 antibody is administered with a coformulation of a PD-1 antagonist, such as a PD-1 antibody, and a CTLA-4 antagonist, such as a CTLA-4 antibody, with the dosages of each component controlled to provide a safe and effective treatment for the subject. In some embodiments, the LILRB2 antibody is administered with a coformulation of pembrolizumab/quavonlimab (MK-1308A; Merck).

Also provided are methods of treating cancer comprising administering a therapeutically effective amount of a LILRB2 antibody product provided herein and a colony-stimulating factor 1 (CSF1) antagonist. Colony-stimulating factor 1 receptor (CSF1R) inhibitors are being developed for cancer therapy. Canarile et al., J Immunother Cancer. 2017 5(a):53. For example, pexidartinib (PLX-3397), has been shown to alter the distribution of tumor-associated macrophages in the tumor microenvironment and promote enrichment of macrophages having an M1-like phenotype. A CSF1 antagonist can be a CSF1 antibody or a CSF1R inhibitor. Such CSF1 antagonists include, for example, pexidartinib, PLX7486, ARRY-382, JNJ-40346527, BLZ945, emactuzumab, AMG820, IMC-CS4, MCS110, PD-0360324, and cabiralizumab.

Also provided are methods of treating cancer comprising administering a therapeutically effective amount of a LILRB2 antibody product provided herein and an agonistic CD40 antibody.

Also provided are methods of treating cancer comprising administering a therapeutically effective amount of a LILRB2 antibody product provided herein and an inhibitory CD47 antibody.

Also provided are methods of treating cancer comprising administering a therapeutically effective amount of a LILRB2 antibody product provided herein and an effective amount of a class Ha histone deacetylase (HDAC) inhibitor, e.g., TMP195.

Also provided are methods of treating cancer comprising administering a therapeutically effective amount of a LILRB2 antibody product provided herein and a TLR7 or TLR8 agonist. The toll-like receptors TLR7 and TLR8 appear to be involved in macrophage polarization in the tumor microenvironment. Agonists of either or both of these receptors can promote functional orientation of tumor-associate macrophages toward M1-like phenotype. It has been reported that resiquimod-loaded β-cyclodextrin nanoparticles have an anti-tumoral effect, which can be enhanced in the presence of a PD-1 antagonist. Rodell et al., Nat Biomed Eng. 2018 2:578-88. Such TLR7/TLR8 agonists include, for example, resiquimod (B848), motolimod (VTX-2337), and imiquimod.

Methods of treating a disease condition in a subject, in which the disease condition is characterized by or mediated through LILRB2 expression by myeloid cells, are also provided. The myeloid cell can be macrophages. The myeloid cells can be osteoclasts or osteoclast precursors. The methods comprise administering to the subject a therapeutically effective amount of an LILRB2 antibody product disclosed herein.

Methods of treatment or prophylaxis of a bone-metabolism disorder in a subject are also provided. The bone-metabolism disorder can be osteoporosis, bone destruction accompanying rheumatoid arthritis, cancerous hypercalcemia, bone destruction accompanying multiple myeloma or cancer metastasis to bone, giant cell tumor, osteopenia, tooth loss due to periodontitis, osteolysis around a prosthetic joint, bone destruction in chronic osteomyelitis, bone Paget's disease, renal osteodystrophy, or osteogenesis imperfecta. The bone metabolism disorder can be osteoporosis. The osteoporosis can be postmenopausal osteoporosis, senile osteoporosis, secondary osteoporosis due to the use of a therapeutic agent such as a steroid or an immunosuppressant, or osteoporosis accompanying rheumatoid arthritis.

### Other Terminology

As used herein, singular forms “a,” “and,” and “the” include plural referents unless the context clearly indicates otherwise. Thus, for example, reference to “an antibody” includes multiple antibodies.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, “can,” “can be,” “may” and “may be” all indicate something contemplated by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

To generate anti-human LILRB2 antibodies from rabbit B cells, rabbits were immunized with human LILRB2 protein. B cells from immunized rabbits were cultured at clonal density, and IgG antibodies in supernatants were evaluated for (a) binding to human and cynomolgus LILRB2 by enzyme-linked immunosorbent assay (ELISA), (b) blocking of LILRB2 binding to HLA-G, (c) binding to cells expressing LILRB2, (d) lack of binding to other LILR(A/B) family members. Variable-regions from positive hits were sequenced, cloned, and expressed as recombinant rabbit/human IgG4 and IgG1 Fc chimeras.

Top clones were selected based on activity in a panel of functional and phenotypic assays using primary human macrophages and T cells, and anti-tumor activity in xenograft model. Select clones were humanized by in silico methods, and humanized antibodies were screened for ability to rescue T cell functional activity (activation and proliferation) from M2c macrophage mediated immune suppression in vitro.

### Example 1: Immunization, Cloning and Initial Screening

The antibody designated B2A was among those cloned from B cells derived from rabbits immunized with human LILRB2 protein. Briefly, two female New Zealand White rabbits were immunized with purified human LILRB2 extracellular domain (ECD)-rabbit Fc fusion protein (SEQ ID NO: 50)(OncoResponse), using a standard immunization method. Rabbits were given a boost each on day 21, day 42, and day 73 following primary immunizations. The pre-immunization bleeds and test bleeds were evaluated for specific antibody titer by indirect ELISA. On day 83 following primary immunization, heparinized whole blood was collected for rabbit monoclonal antibody development. B cells in peripheral blood from both rabbits were collected after last boost, then isolated, purified, and cultured at clonal density using proprietary methods. Bio-panning was performed using human LILRB2 ECD-human Fc fusion protein (SEQ ID NO: 51) (OncoResponse) B cell culture supernatants from 80 96-well plates were transferred to ELISA plates coated with human-Fc LILRB2 ECD fusion protein. An indirect ELISA was performed probing with secondary anti rabbit-IgG antibody (ImmunoPrecise). For the recovery of antibodies specific to target antigen, negative screening of the positive candidates was performed against an unrelated human-Fc fusion protein (ImmunoPrecise). B cell supernatants were also evaluated for ability to block LILRB2-Fc (R&D Systems, No. 2078-T4) binding to HLA-G. The top 96 responding wells were preserved in a standard RNA lysis buffer (ImmunoPrecise) for antibody RNA isolation and generation of recombinant plasmid DNA.

The rabbit antibody heavy and light (kappa) chain variable regions were cloned into separate mammalian expression vectors containing the human IgG heavy and kappa constant regions for top ranked positive clones. Recombinant monoclonal antibodies were generated using transfected HEK-293 cells, purified using standard methods and evaluated in several biochemical and cell-based functional assays.

### Example 2: Binding of Chimeric Antibodies to LILRB2 by ELISA (Human and Cynomolgus)

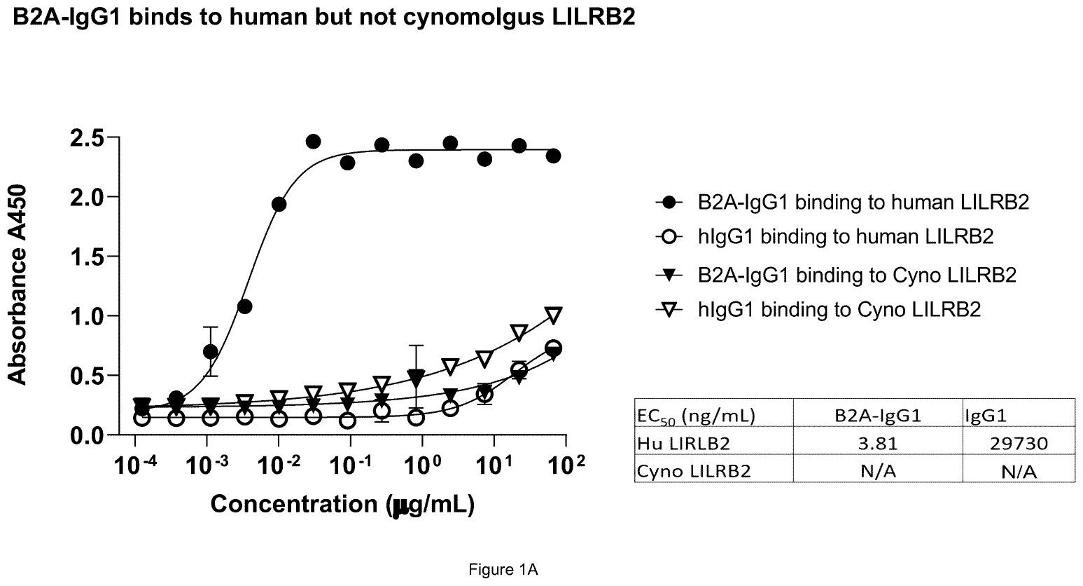

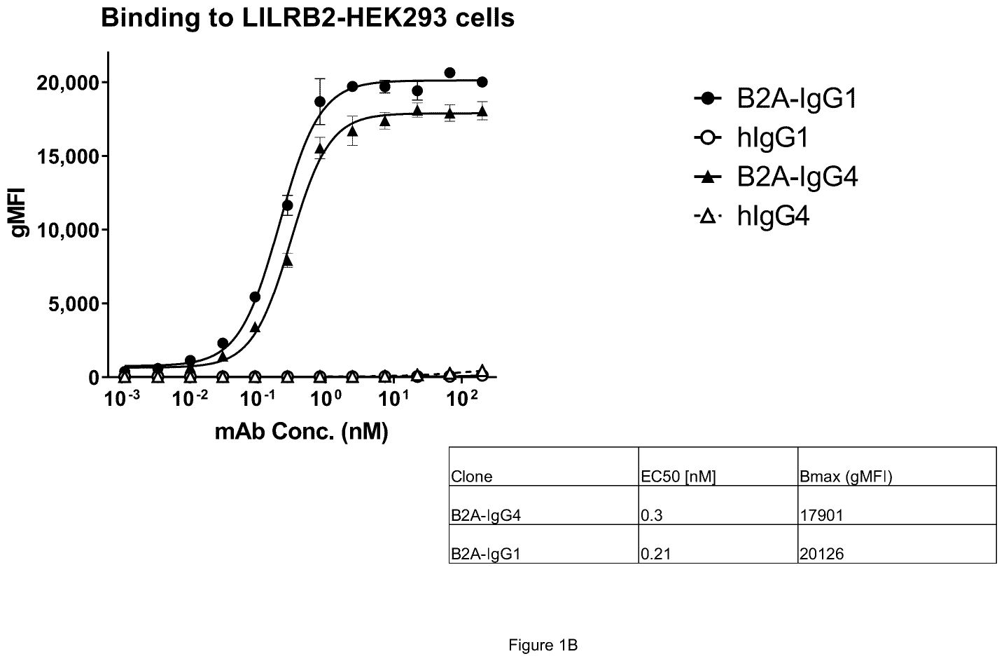

To confirm binding to human and cynomolgus LILRB2, antibodies in supernatants were evaluated for binding by enzyme-linked immunosorbent assay (ELISA). Recombinant LILRB2 proteins (human: SEQ ID NO: 48; cynomolgus) were diluted in PBS to 2 μg/mL and added to 384-well high-binding ELISA plates (Greiner Bio-One Microlon™) at 25 μL/well and incubated at 4° C. overnight. The plates were washed three times with Wash Buffer (0.05% Tween® 20 in PBS), using a microplate washer and then blocked with 90 μL/well of ELISA Blocking Buffer A (1% BSA in PBS) for 1 hour at room temperature (RT). After blocking, 25 μL/well of LILRB2 antibodies or isotype control were added to the plates and incubated for 1 hour at RT. After primary antibody binding, plates were washed three times with Wash Buffer using a microplate washer. Secondary detection antibody (HRP-goat anti-rabbit Fab fragment) was diluted to 1:3000 in assay diluent (1% BSA in PBS) and 25 μL/well was added to plates and incubated at RT protected from light for 1 hour. Following incubation, plates were washed four times with Wash Buffer, using a microplate washer. After removal of the final wash, 25 μL/well of 1-Step™ Ultra TMB-ELISA Substrate Solution (Thermo Fisher, No. 34028) was added and plates were incubated for 5-10 min at RT, protected from light. After development, the reaction was stopped by addition of 25 μL/well of 0.3 M HCl, and plates were read using an EnVision (Perkin Elmer) microplate reader at 450 nm. EC₅₀ values were calculated based on log concentration of the primary antibodies at optical density at 450 nm. Binding of anti-LILRB2 B2A-IgG1 to human and cynomolgus LILRB2 is presented in FIG. 1a, showing binding to human LILRB2, with an EC₅₀ of 3.81 ng/mL, but no binding to cynomolgus LILRB2.

### Example 3: Preparation of Cells Expressing Human LILRB2

Cells stably expressing human LILRB2 were generated. Human embryonic kidney cells (HEK293T/17; ATCC, CRL-11268) were cultured per ATCC guidelines in DMEM media (Gibco, No. 11965-084) supplemented with 10% fetal bovine serum (FBS; HyClone, No. SH30396.03; heat-inactivated before use). Pre-packaged lentiviral particles containing puromycin-selectable lentivector backbone were purchased from G&P Biosciences: without the gene of interest (negative control; No. LTV0001) or with human LILRB2 (SEQ ID NO: 52) (No. LTV2992). HEK293T cells (5×10⁴) were transduced with human LILRB2 lentiviral particles at multiplicity of infection (MOI) of 10, in the presence of 8 μg/mL polybrene infection/transfection reagent (Millipore, No. TR-1003-G), for 24 hours at 37° C., 5% CO₂. Virus-containing media was then removed, and cells were allowed to recover in fresh media for two days prior to selection in media containing 0.75 μg/mL of puromycin (Thermo Fisher, No. A1113803). By selecting for stable LILRB2 expression based on puromycin as a selection marker, populations of LILRB2-expressing cells were isolated and cultured. Expression of LILRB2 on puromycin-resistant cell populations was confirmed by flow cytometry using BD FACSymphony™ cytometer and LILRB2-specific antibodies. Alongside the LILRB2 transductions, cells were also transduced with negative control lentivirus particles, using the same protocol, to generate a stable, puromycin-resistant control cell line (contains lentivector backbone without the gene insert) for use in assays.

Once target expression was confirmed, the cells were expanded. The selected polyclonal pool of cells was banked at 4-5 million cells/mL/vial and master stocks were made. Banking of cells involves pelleting cells from culture by centrifugation, removing the medium, and resuspending cells in 4° C. DMEM+10% FBS first, then adding an equal volume of 2×ATCC recommended freezing medium (DMEM+10% FBS plus 10% DMSO) such that cells were at a density of 4-5 million/mL in 1× freezing medium (DMEM+10% FBS plus 5% DMSO). Aliquots of the cell suspension were placed into a 4° C. Mr. Frosty™ alcohol-based slow freezing system and immediately stored at −80° C. These frozen cell aliquots were stored at −80° C. for one day and then were moved to liquid nitrogen vapor phase tank.

For use in assays, a master stock vial was thawed in a 37° C. water bath for one minute and centrifuged to pellet cells. Cells were suspended in DMEM+10% FBS and expression of target protein was checked by flow cytometry. These cells were expanded in culture to make 30 working stocks and expression of target protein was checked by flow cytometry. These cells were expanded in culture to make 30 working stocks.

### Example 4: LILRB2 Antibodies Bind to Cells Expressing Human LILRB2

To confirm binding of LILRB2 antibodies, flow cytometry binding assays were performed using HEK293T cells stably expressing human LILRB2 (Example 3). The modified HEK293T cells were incubated at 4° C. for 30 min in Blocking Buffer B (FACS Buffer (PBS, 1% FBS+0.05% NaN₃ (Ricca Chemical, No. 7144.8-16, diluted), 2 mM EDTA) containing 10% FBS to block nonspecific binding. Titrations of LILRB2 antibodies and isotype control antibodies (OncoResponse) conjugated to AF647 (Thermo Fisher, No. 20106) were directly added on top of Blocking Buffer B to the HEK293T cells, then incubated at 4° C. for 1 hour. Cells were washed 2× in FACS Buffer and then incubated with AF647-goat anti-human IgG Fc (Jackson ImmunoResearch, No. 109-605-098) for 30 min. Cells were washed 2× in FACS Buffer and then stained with for 10 min with Zombie Violet fixable viability dye (BioLegend, No. 423114) at RT, washed with FACS Buffer and resuspend in 100 μL of FACS Buffer for acquisition on a BD FACSymphony™ or BD FACSCanto™ II flow cytometer (BD Biosciences). The mean fluorescence intensity (MFI) binding of LILRB2 antibodies was gated on live cells using FlowJO software (10.5.3, FlowJO, LLC) and GraphPad prism for EC₅₀ binding calculations. The B2A-IgG1 and B2A-IgG4 chimeras bound the LILRB2-expressing HEK293T cells, with equivalent EC₅₀ values of 0.21 nM and 0.30 nM respectively. A representative example is shown in FIG. 1B.

### Example 5: LILRB2 Antibodies do not Bind to Other LILRB or LILRA Family Members

To determine that the anti-LILRB2 antibodies specifically bind to LILRB2 and not other LILRB and LILRA family members, a cross-reactivity assay was performed. Cross-reactivity of the LILRB2 antibodies to other LILRB proteins was assessed by antibody binding to transiently transfected HEK293-6E cells, measured by flow cytometry. Binding was evaluated to HEK293-6E cells transiently transfected with human LILRB-1, -2, -3, -4, and 5 plasmids (Origene, Nos. RC219949, RC217935, RC211228, RC220932, RC206516) or to cells transiently transfected with human LILRA-1, -2, -4, -5, and -6 plasmids (Origene, Nos. RC210808, RC205626, RC220452, RC212310 RC212965). AF647 mouse anti-human IgG-Fc secondary antibody (Jackson ImmunoResearch, No. 109-605-098) was used for the detection of bound human anti-LILRB2 antibodies on cells. Murine primary antibodies to LILRA and LILRB targets and corresponding isotype controls were used to confirm specific binding of all targets tested (R&D Systems) while an AF647 F(ab′)₂ fragment goat anti-mouse antibody (Jackson ImmunoResearch, No. 115-606-062) was used for the detection of bound positive control antibodies. Binding of B2A-IgG1 and B2A-IgG4 to other LILRA and LILRB family members is presented in Tables 1A and 1B. No binding of B2A-IgG4 or B2A-IgG1 to LILRB-1, -3, -4, or -5 was observed, and no binding of either antibody to LILRA-1, -2, -4, -5, or -6 was observed, confirming specificity of these antibodies.

Tables 1A and 1B—Flow Cytometry Binding of B2A-IgG1 and B2A-IgG4 to Other LILR Family Members with Select Chimeric Clones

| | Vector | | | | | |
|---|---|---|---|---|---|---|
| | Control | hLILRA1 | hLILRA2 | hLILRA4 | hLILRA5 | hLILRA6 |
| mAb | (MFI) | (MFI) | (MFI) | (MFI) | (MFI) | (MFI) |
| B2A-IgG4 | 117 | 73 | 31 | 30 | 21 | 22 |
| B2A-IgG1 | 117 | 898 | 38 | 116 | 127 | 223 |
| αLILRA1 [mIgG2B] | | 6496 | | | | |
| αLILRA2 [mIgG1] | | | 3813 | | | |
| αLILRA4 [mIgG2A] | | | | 2445 | | |
| αLILRA5 [mIgG2A] | | | | | 1441 | |
| αLILRA6 [mIgG1] | | | | | | 1509 |
| hIgG4 | | | | | | |
| mIgG2A | | | | 105 | 127 | |
| mIgG2B | | 333 | | | | |
| mIgG1 | | | 38 | | | 169 |

| | Vector | | | | | |
|---|---|---|---|---|---|---|
| | Control | hLILRB1 | hLILRB2 | hLILRB3 | hLILRB4 | hLILRB5 |
| mAb | (MFI) | (MFI) | (MFI) | (MFI) | (MFI) | (MFI) |
| B2A-IgG4 | 117 | | 1163 | 22 | 20 | 21 |
| B2A-IgG1 | 117 | 43 | 11285 | 163 | 40 | 147 |
| αLILRB2B2Comp1 | | | 9632 | | | |
| αLILRB3 | | | | 1311 | | |
| [mIgG2A] | | | | | | |
| αLILRB4 | | | | | 7441 | |
| [mIgG2A] | | | | | | |
| αLILRB5 | | | | | | 3195 |
| [mIgG2B] | | | | | | |
| hIgG4 | | | 29 | | | |
| mIgG2A | | | | 81 | 43 | |
| mIgG2B | | 49 | | | | 119 |
| mIgG1 | | | | | | |

### Example 6: LILRB2 Antibodies Block LILRB2-Fc Binding to ANGPTL-2 and -5

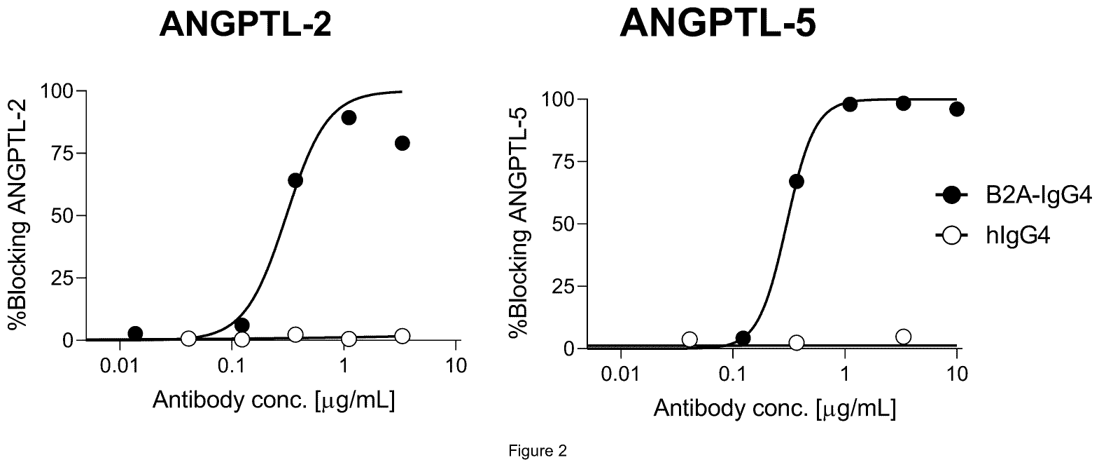

Angiopoietins and Angiopoietin-like (ANGPTL) proteins are secreted glycoproteins that play roles in angiogenesis, lipid metabolism, hematopoietic stem cell expansion, and inflammation. Angiopoietins signal through the tyrosine kinase receptors Tie1 or Tie2, whereas ANGPTL proteins are considered orphan ligands. ANGPTL-1, -2, -5, and -7 were shown to bind to human leukocyte immunoglobulin like receptor B2 (LILRB2). To determine if anti-LILRB2 antibodies block the binding of ANGPTL-2 and -5 to human LILRB2, blocking ELISA assays were performed. Proteins were purchased from R&D Systems (Nos. 9795-AN-050, 6675-AN-025/CF) and reconstituted in PBS or water. 384-Well plates were coated with ANGPTL-2 or ANGPTL-5 at a concentration of 5 μg/mL and sealed for overnight incubation at 4° C. Following incubation, plates were rinsed with Wash Buffer, then blocked with Blocking Buffer C (3% BSA diluted+0.05% Tween® 20 in PBS) for 2 hours at 37° C. Anti-LILRB2 antibodies were incubated with LILRB2-Fc protein for 1 hour at RT. Blocking Buffer C was removed from plates, then anti-LILRB2 antibody titrations/LILRB2-Fc mixtures were incubated in wells overnight at 4° C. Plates were washed with Wash Buffer, then goat anti-human IgG Fc biotinylated secondary antibody was diluted in assay buffer and added to wells. Secondary antibody was incubated onto wells for 2 hours at RT, then rinsed with Wash Buffer. Streptavidin-HRP was diluted per manufacturer's instructions in assay buffer and incubated onto wells for 30 minutes at RT in the dark. Plates were rinsed with Wash Buffer then manufacturer reagents were added at 1:1 dilution and incubated onto wells for 7.5 minutes at RT. Stop solution was added and absorbance was measured at 450 nm. As shown in FIG. 2, B2A-IgG4 blocked the binding of LILRB2-Fc to ANGPTL-2 and -5 in a dose-dependent manner.

### Example 7A: LILRB2 Antibody B2A-IgG4 Blocks LILRB2-Fc Binding to HLA-G

Tumor-associated HLA-G is an HLA Class I ligand for LILRB2. Binding of HLA-G to LILRB2 induces an immune suppressive signal in LILRB2 expressing myeloid cells. To determine if the anti-LILRB2 antibodies block the HLA-G-LILRB2 interaction and prevent the induction of an inhibitory signal, blocking ELISA assays in a dose titration were performed. 384-well plates were coated with monomer HLA-G (Fred Hutchinson Cancer Research Center, No. bHLA-G) at a concentration of 5 μg/mL, diluted in PBS, then plates were incubated at RT for 1 hour. Plates were rinsed with Wash Buffer three times, then blocked with Blocking Buffer A for 30 minutes at RT. During blocking incubation step, LILRB2-Fc protein and antibody titrations were prepared in Blocking Buffer A. LILRB2-Fc protein was then incubated with antibody titrations for 30 minutes at RT to allow for binding prior to addition onto plate. Plates were rinsed three times with Wash Buffer, then 25 μL of protein/antibody mixture was added per well and incubated for 1 hour at RT. Following antibody incubation, plates were washed three times with Wash Buffer. Anti-human IgG Fc-HRP secondary antibody was diluted and added at 25 μL/well. Plates were incubated in the dark for 1 hour at RT. Following secondary antibody incubation, plates were washed four times with Wash Buffer. 25 μL/well ultra-neat TMB was added per well for development and incubated for 5 minutes at RT. Reaction was stopped using 0.3 M HCl (25 μL/well). Absorbance was read at 450 nM using the Envision. The IC₅₀ for HLA-G blocking by clone B2A-IgG4 was 0.066 μg/mL.

### Example 7B: LILRB2 Antibody B2A-IgG4 Blocks LILRB2-Fc Binding to HLA-G Expressed on Tumor Cells

Binding of LILRB2 on macrophages to HLA-G on cancer cells enhances immunosuppressive functions of myeloid cells. The ability of the LILRB2 antibodies to block HLA-G binding on cancer cells is crucial to the efficacy of anti-LILRB2 antibodies. LILRB2 antibodies were evaluated for blocking the binding of recombinant LILRB2-Fc protein to HLA-G expressed on 721.221 B lymphoma cells by flow cytometry.

Recombinant human LILRB2-Fc-Avitag protein (Acro Biosystems, No. L12-H82F5) was diluted to 30 μg/mL in FACS Buffer (PBS containing 2 mM EDTA+1% FBS+0.05% NaN₃) and combined in equal parts volume with anti-LILRB2 antibodies at concentrations of 40, 20, 10, or 5 μg/mL, then incubated at 4° C. for 1 hour. B cells (721.221) were washed with PBS and counted, then resuspended at a cell density of 1.3×10⁶/mL (50×10³ cells/well) in Fc Block (10% NGS (Sigma, No. G6767), 2.5% FBS, 1% anti-CD32 (BD Biosciences, No. L12-H82F5), 2 mM EDTA, 0.05% NaN₃ in PBS) in FACS Buffer, then incubated at RT for 30 minutes. Cells (50 μL) were added to wells containing LILRB2-Fc-Avitag protein/antibody pre-incubations, for a final concentration of 7.5 μg/mL LILRB2-Fc-Avitag protein and 20, 10, 5, or 2.5 μg/mL antibody, then incubated for 1 hour at RT. The cells were then washed with FACS Buffer (100 μL/well) and the plates were centrifuged at 450×g for 5 minutes to pellet. Supernatant was removed and a second wash with FACS Buffer at 250 μL per well was used, then centrifuged again at 450×g for 5 minutes to pellet.

Streptavidin-PE (BioLegend, No. 405204) at a 1:250 dilution (75 μL/well) was added and the cell/antibody mixture was incubated for 30 minutes at RT, protected from light. Cells were then washed twice in PBS containing 1 mM EDTA at 200 μL/well volume, then centrifuged at 450×g for 5 minutes to pellet cells. Cells were resuspended in 50 μL cell viability dye (Zombie violet, BioLegend, No. 423114) at a 1:2000 dilution, and incubated for 10 minutes at RT, protected from light. A final wash of 200 μL/well FACS Buffer was performed, then cells were pelleted by centrifugation at 450×g for 5 minutes. Supernatant was removed and cells were resuspended in 100 μL FACS Buffer for flow cytometry analysis, as described in Example 4. The B2A-IgG4 clone blocked the binding of LILRB2-Fc to HLA-G-expressing 721.221 cells in a dose-dependent manner, whereas the IgG4 isotype control did not affect LILRB2-Fc binding to 721.221 cells. Complete inhibition of binding by the B2A-IgG4 clone was observed at concentrations of 20 and 10 μg/mL and partial blocking of 82% and 32% (normalized) was observed at concentrations of 5 and 2.5 μg/mL, respectively.

### Example 8: Binning of LILRB2 Antibodies

Anti-LILRB2 antibodies were evaluated in blocking flow cytometry experiments with HEK293T-LILRB2 cells stably expressing LILRB2 (Example 3) and biotin-conjugated benchmark LILRB2 antibodies prepared based on literature: B2Comp1 (PCT publication WO 2021/138079 A1, there designated MK-4830) and B2Comp2 (US patent publication US 2019/0194327 A1, there designated J-19.h1). The variable domains of these antibodies were cloned into IgG4 and lambda (B2Comp1) or kappa (B2Comp2) constant regions. LILRB2 flow cytometry binning categorizes antibodies into different bins according to the ability to block the binding of the benchmark antibodies to HEK293T-LILRB2 cells. Briefly, HEK293T-LILRB2 cells were blocked with Blocking Buffer B for 20 minutes at 4° C. Following incubation with Blocking Buffer B, “cold” blocking anti-LILRB2 antibodies in FACS Buffer were added to the HEK293T-LILRB2 cells for a final concentration of 0.1, 1, or 10 μg/mL, then incubated for 1 hour at RT. Then, the “hot” biotinylated benchmark antibody was added for a final assay concentration of 5 ng/mL. Following the 45 minute incubation at RT, cells were washed in FACS Buffer and incubated with Streptavidin-APC (Invitrogen, No. S32357) to quantify binding of the benchmark to HEK293T-LILRB2 cells for 45 minutes at RT. Cells were then washed in FACS Buffer and stained with cell viability dye eFluor™ 780 (e780; Thermo Fisher, No. 65-0865-14) for 15 min at RT. Cells were washed with FACS Buffer and resuspended in 100 μL FACS Buffer for acquisition by flow cytometry as described in Example 4.

Chimeric anti-LILRB2 antibodies were binned into 4 separate categories:

Bin 1: antibodies that block the binding of B2Comp1 and B2Comp2 to HEK293T-LILRB2 cells and share an epitope for with both B2Comp1 and B2Comp2

Bin 2: antibodies that block the binding of B2Comp1 but not B2Comp2 to HEK293T-LILRB2 cells and share an epitope with B2Comp1

Bin 3: antibodies that neither block binding of B2Comp1 nor B2Comp2 to HEK293T-LILRB2 cells

Bin 4 antibodies that do block the binding of B2Comp2 but not B2Comp1 to HEK293T-LILRB2 cells and share an epitope with B2Comp2

The binning data are shown in Table 2. B2A-IgG4 did not block the binding of either of the comparator LILRB2 antibodies to HEK293T-LILRB2 cells at 0.1, 1 and 10 μg/mL, indicating that B2A-IgG4 does not share an epitope with B2Comp1 or B2Comp2 (Bin 3). This categorizes the anti-LILRB2 antibodies described herein as having novel epitopes that are not shared with certain other published anti-LILRB2 antibodies.

TABLE 2
B2A-IgG4 does not block the binding of benchmark antibodies

| | % Blocking of B2Comp1 Binding | | | % Blocking of B2Comp2 Binding | | |
|---|---|---|---|---|---|---|
| Blocking mAb | 0.1 μg/mL | 1 μg/mL | 10 μg/mL | 0.1 μg/mL | 1 μg/mL | 10 μg/mL |
| B2Comp1 | 43 | 95 | 99 | 37 | 73 | 77 |
| B2Comp2 | 47 | 78 | 80 | 44 | 97 | 100 |
| B2A-IgG4 | 0 | 0 | 0 | 0 | 0 | 0 |
| hIgG4 | 0 | 0 | 0 | 0 | 0 | 0 |

### Example 9: Isolation and Differentiation of Human Primary Cells

The evaluation of anti-LILRB2 antibodies in immunological assays requires the isolation of human T cells and monocytes, as well as the differentiation of monocytes into immune suppressive macrophages. Various techniques are known in the art, such as that described below for cells used in these examples. Apheresis products are collected from subjects and autologous monocytes and T cells are isolated using the technique described or another technique commonly used in the art. Briefly, human monocytes and T cells are isolated from white blood cells (WBCs) according to standard techniques. (LeukoPak, No. 4510-01 Full LeukoPak, BloodWorks Northwest, Seattle, WA). Peripheral blood mononuclear cells (PBMCs) are purified from LeukoPaks by standard density gradient centrifugation (FicollPaque® Premium 1.073, or 1.077, GE Healthcare, No. 17-5449-52, or Cytiva No. 17144003). The supernatant is discarded, and the pellet resuspended in 20 mL EasySep™ Buffer (STEMCELL Technologies, No. 20144) for counting of PBMCs and for further isolation of monocytes and T cells. Monocytes are isolated using the EasySep™ Human Monocyte Isolation kit (STEMCELL Technologies, No. 19359) following the manufacturer's instructions. Total CD3+, CD4+ and CD8+ T cells are isolated using the EasySep™ Human CD3+, CD4+ and CD8+ T Cell Isolation kits (STEMCELL Technologies, Nos. 19051, 17952, and 17953, respectively), following the manufacturer's instructions. These negative selection kits use antibodies to label undesired cell types for removal, allowing the desired target cells to be isolated from the sample untouched.

### Example 10: Macrophage Generation

Macrophages can be generated from PBMC-derived monocytes with commonly used techniques, such as that described below.

Generation of M0 Macrophages: At day 0, monocytes from individual subjects (isolated as described in Example 9) were plated in 96-well culture plates (Thermo Fisher (Costar), No. 09-761-175), at 25-50×10³ cells/100 μL/well in M0 culture medium (90% X-VIVO™ 15+10% FBS+100 ng/mL human M-CSF (PeproTech, No. 300-25)). Cells were incubated at 37° C. and 5% CO₂ for 5 to 6 days to produce M0 macrophages.

Generation of immune suppressive M2c macrophages: At Day 5 of culture, M0 macrophages were polarized to M2c macrophages by aspirating the medium gently from each plate and replacing it with 100 μL/well of M2c culture medium (M0 culture medium+20 ng/mL human IL-10 (PeproTech, No. 200-10)). Cells were incubated at 37° C. and 5% CO₂ for 2 days. At Day 7-8 of culture, the M2c macrophages were ready for coculture assay setup. M2c macrophages were detached from the plate by incubation with Macrophage Detachment Solution DXF (PromoCell) and washed in PBS prior to downstream assays.

### Example 11: Generation of Exhausted T Cells

Exhausted T cells are an indicator of an immune-suppressive tumor microenvironment and contribute to cancer immune evasion. To mimic exhausted T cells in the TME, exhausted T cells were generated by repeated stimulation. Exhausted T cells having a blast-like morphology were generated from human PBMCs by repeated (3×) phytohemagglutinin (PHA) stimulation. Cells were counted and incubated at 1×10⁶ cells/mL in T cell blast culture medium (90% IMDM (Thermo Fisher (Gibco), No. 12440053)+10% human serum+2 μg/mL PHA-L (Sigma-Aldrich (Roche), No. 11249738001)+4 ng/mL recombinant human IL-2, (R&D Systems, No. 202-IL)). Cells were split 1:2 or 1:3 every 3 to 4 days and cultured for 10 days total (2 splits over 10 days; 3 total PHA stimulations). Fresh T cell blast culture medium was added to the cells at each split. The cells were harvested on day 10 and either set up for coculture assays or frozen for future use. Exhausted T cell phenotype was confirmed by expression of PD-1, TIM-3, and TIGIT, as well as the transcription factor Eomes (data not shown).

### Example 12: LILRB2 Antibodies Bind to Human Monocytes and M0 and M2c Macrophages

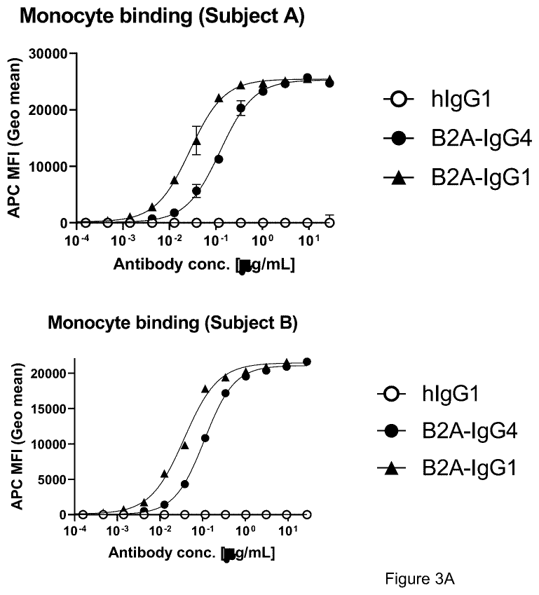

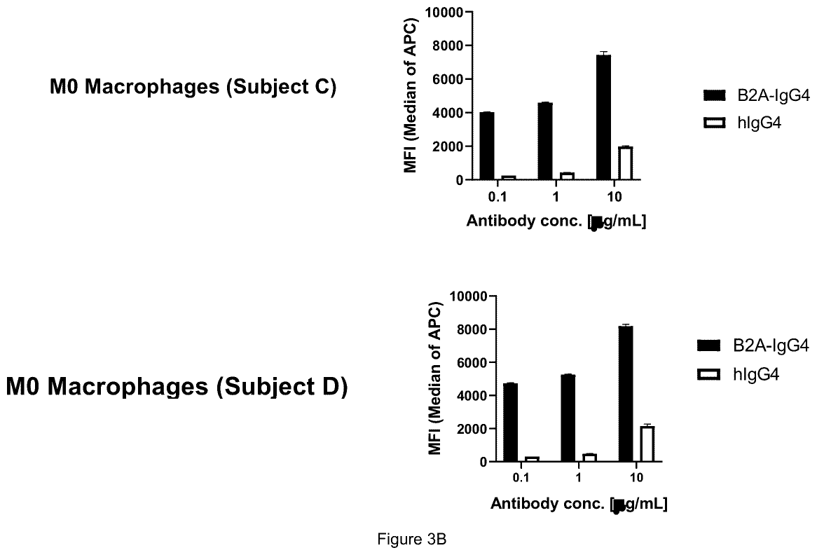

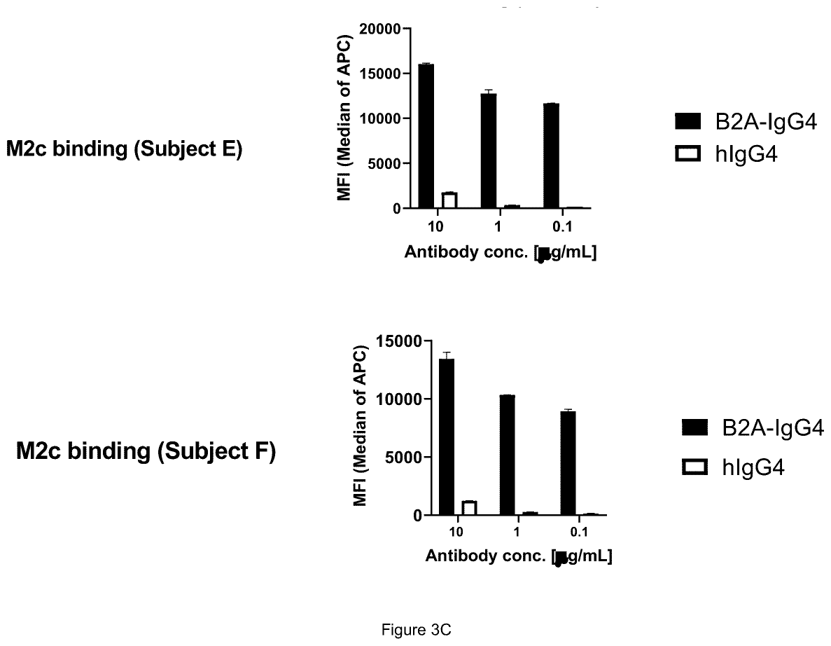

Anti-LILRB2 antibodies were evaluated for their ability to bind myeloid cell subsets expressing LILRB2, including monocytes, M0 macrophages, and M2c macrophages. M2c macrophages are used as a surrogate for tumor-associated macrophages, a suppressive macrophage commonly found in the tumor microenvironment. Frozen monocytes were removed from storage in vapor phase liquid nitrogen and thawed by gentle swirling in a water bath at 37° C., then resuspended in X-VIVO™ 15 media with 10% FBS. PBMC were placed in a 15 mL conical tube and centrifuged at 300×g for 5 minutes, then supernatant was removed. Cells were resuspended in Assay Medium (X-VIVO™ 15+10% FBS) at a cell density of 2.5×10⁵ cells/mL. Cells were plated into 96-well flat bottom plates at 100 μL/well (25K cells/well). M0 and M2c macrophages were generated as described in Example 10 and collected from flasks by incubation for 15 min at RT in Macrophage Detachment Solution DXF, then removed from the flask into X-VIVO™ 15 medium. Following centrifugation, the cells were resuspended in FACS Blocking Buffer D (FACS Buffer+10% FBS+0.5 mg/mL human IgG1) then incubated at 4° C. for 30 minutes. Cells in 25 μL FACS Blocking Buffer D were transferred to a 384-well plate at 2.5×10⁴ cells/well and 25 μL of titrated AF647-conjugated LILRB2 antibodies or AF647-conjugated IgG1 isotype control were added directly to each well at 2× final assay concentration. Cells were incubated with antibodies at 4° C. for 1 hour. Cells were washed two times with FACS Buffer, stained with Zombie UV live/dead stain viability dye (BioLegend, No. 423107) (1:500 dilution) for 15 min at RT in the dark, washed with FACS Buffer and resuspended in 200 μL FACS Buffer for acquisition by flow cytometry as described in Example 4. GraphPad prism was used for EC₅₀ binding calculations. B2A-IgG1 and B2A-IgG4 bind to human monocytes and M0 and M2c macrophages (FIG. 3).

### Example 13: LILRB2 Antibodies Enhanced IFN-γ Response in LPS-Stimulated PBMC

To determine if LILRB2 blockade boosts the innate immune response, which in turn enhances IFN-γ secretion to abate the immune suppressive TME, the effect of B2A-IgG1 and IgG4 chimeras on the IFN-γ secretion of PBMC in response to lipopolysaccharide (LPS) was evaluated. Frozen PBMC were removed from storage in vapor phase liquid nitrogen and thawed by gentle swirling in a water bath at 37° C., then resuspended in Assay Medium. PBMC were placed in a 15 mL conical tube and centrifuged at 300×g for 5 minutes, then supernatant was removed. Cells were resuspended in Assay Medium at 3×10⁶ cells/mL. Cells were plated into 96-well flat bottom plates at 100 μL/well (300K cells/well). This cell density is optimized for the average level of IFN-γ response per subject. Fewer cells were plated per each well if the subject was known to yield a higher cytokine response. Cells were rested at 37° C., 5% CO₂ for 1 hour.

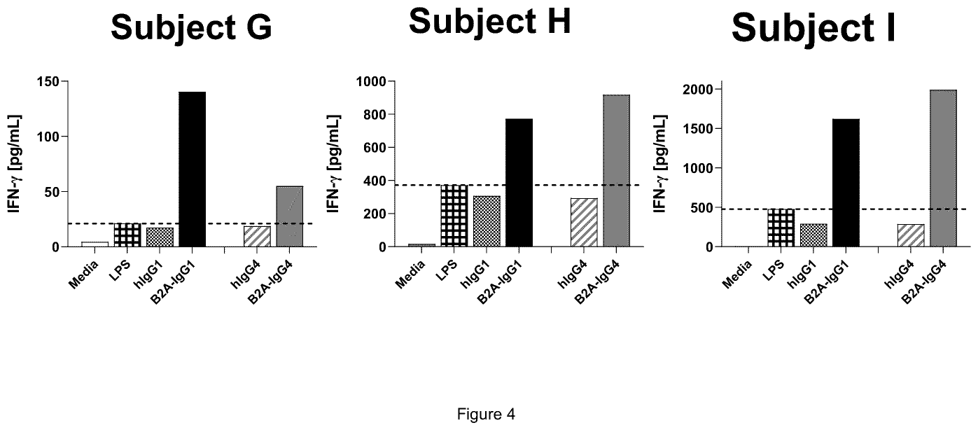

The LILRB2 antibodies were diluted to a 4× concentration (final concentration of 1 μg/mL) in Assay Medium and 50 μL/well diluted antibody was added. For wells that did not receive antibody, 50 μL/well of Assay Medium was added. Cells with antibody dilutions were incubated at 37° C., 5% CO₂ for 2 hours prior to LPS stimulation. LPS was diluted in Assay Medium to a 4× concentration (final concentration of 1 μg/mL in assay), and 50 μL/well was added to the cell/antibody mixtures, except that for subject samples that had a known higher IFN-γ response, LPS final concentration was 0.1 μg/mL. For control wells (no LPS), 50 μL/well of Assay Medium was added. Cells were incubated at 37° C., 5% CO₂ for 24 hours, at a final volume of 200 μL/well. Supernatant (150 μL) was harvested following 24-hour incubation and either frozen at −80° C. or directly tested for IFN-γ secretion by ELISA (R&D Systems). Anti-LILRB2 antibodies contributed to a pro-inflammatory phenotype by displaying enhanced IFN-γ secretion from PBMC stimulated with LPS. Both B2A-IgG1 and B2A-IgG4 elicited similar IFN-γ secretion profiles, shown in three representative subjects in FIG. 4.

### Example 14: LILRB2 Antibodies Enhanced TNF-α Secretion in CD40-Activated Macrophages

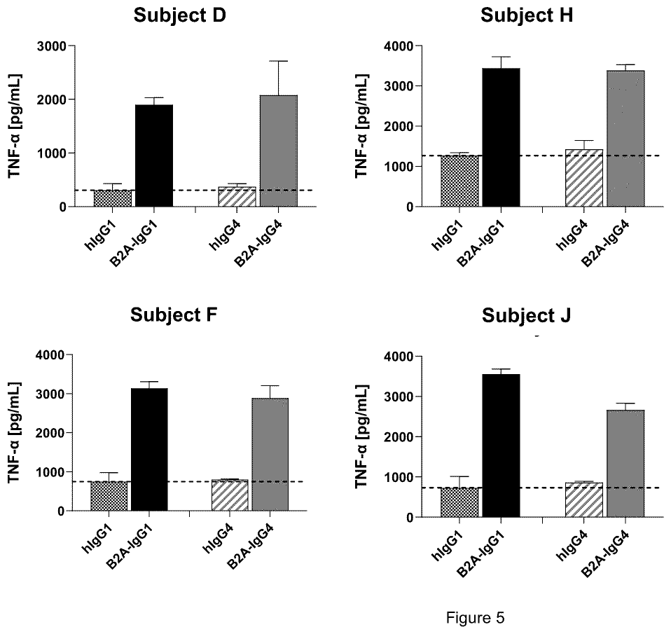

CD40L expressed on T cells is the primary molecule responsible for the activation of macrophages in the TME, through binding to CD40 expressed on macrophages via cell-to-cell contact. CD40/CD40L interactions are important for activating macrophages to behave as effector cells that mediate inflammation in T cell-mediated inflammatory processes. An assay was developed to assess inflammatory cytokine production by macrophages treated with LILRB2 antibodies, but without the presence of T cells. A HEK293 cell line modified to express CD40L (CrownBio, No. C2041) was used to mimic the binding and subsequent stimulation by an activated T cell. M0 macrophages were generated from monocytes (25×10³/well) as described in Example 10. Medium was removed from the wells and 100 μL/well fresh Assay Medium was added. LILRB2 antibodies (B2A-IgG1 and B2A-IgG4) at a 5× concentration were added at 50 μL/well, and incubated onto macrophages at 37° C., 5% CO₂ for 2 hours. During the 2-hour incubation, CD40L-expressing HEK293 cells were harvested from flasks, and irradiated at 40 Gy. Following pre-incubation with LILRB2 antibodies, CD40L-expressing HEK293 cells (5×10³) were added at a volume of 100 μL/well, bringing the final well volume to 250 μL/well in 96-well plates, and incubated overnight at 37° C., 5% CO₂. Following incubation, 200 μL of supernatant was harvested from wells and either frozen at −80° C. or immediately assayed for TNF-α secretion by HTRF according to manufacturer instructions (CisBio, No. 62HTNFAPET). Both B2A-IgG1 and B2A-IgG4 show comparable potentiation of TNF-α secretion by CD40L-activated macrophages, shown in four different subjects (FIG. 5).

### Example 15: LILRB2 Antibodies Relieve M2c-Mediated Immune Suppression in M2c/CD8+ or M2c/CD4+ T Cell Cocultures

The crosstalk between LILRB2-expressing immunosuppressive myeloid cells and T cells contributes to T cell exhaustion and the lack of anti-tumor immune response in the TME. This crosstalk can be modeled by in vitro co-culture of autologous monocyte-derived M2c macrophages and anti-CD3 stimulated CD8+ or CD4+ T cells. Reduction of immune suppression can be evaluated by quantitation of T cell proliferation, and IFN-γ and perforin secretion as surrogates of T cell activation and anti-tumor activity. After polarization of M0 macrophages to M2c macrophages as described in Example 10, supernatants were removed from the macrophages in 96-well culture plates and replaced with 100 μL Assay Medium containing a final concentration of 0.25 or 0.63 μg/mL OKT3 (BioLegend #317326. LILRB2 antibodies (a dose titration from 20-0.0015 μg/mL) were added at a volume of 50 μL/well, then plates were incubated at 37° C., 5% CO₂ for 1-2 hours. While the M2c macrophages were incubated with the antibodies, autologous CD4+ or CD8+ T cells were isolated from PBMCs as described in Example 9. Isolated CD8+ or CD4+ T cells were labeled with CellTrace™ violet (Thermo Fisher, No. C34557) as described in Example 7B. Excess CellTrace™ was washed off with pre-warmed Assay Medium and labeled CD8+ or CD4+ T cells were resuspended in Assay Medium, then added to the M2c/antibodies preparation at 5×10⁵ cells/mL in volume of 100 μL at 1:1 ratio of M2c:CD4+ or M2c:CD8+ T cells. Cells were then incubated at 37° C., 5% CO₂ for 72 hours.

Supernatants containing T cells were transferred to a V-bottom 96-well plate, centrifuged to pellet the T cells, and culture supernatants were collected and frozen at −80° C. for quantification of human IFN-γ and perforin levels by MSD-ELISA (Meso Scale Discovery, custom U plex assay). T cell pellets were stained with e780 viability dye for 10 min at RT in the dark, washed with 150 μL FACS buffer, and resuspended in 100 μL of FACS Buffer for acquisition on BD FACSymphony™ or FACSCanto™ flow cytometer (BD Biosciences). The percentage and overall count of proliferating CD8+ or CD4+ T cells was analyzed using FlowJO software and reported as percent CellTrace+ dividing cells or total count of CellTrace+ dividing cells.

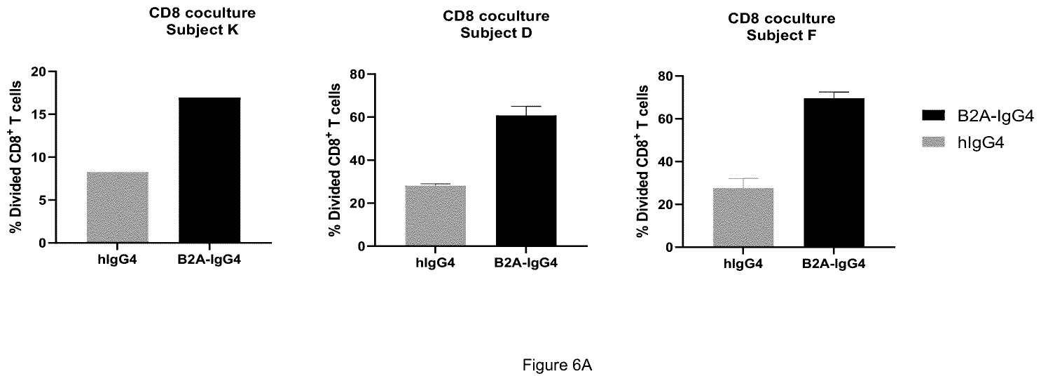

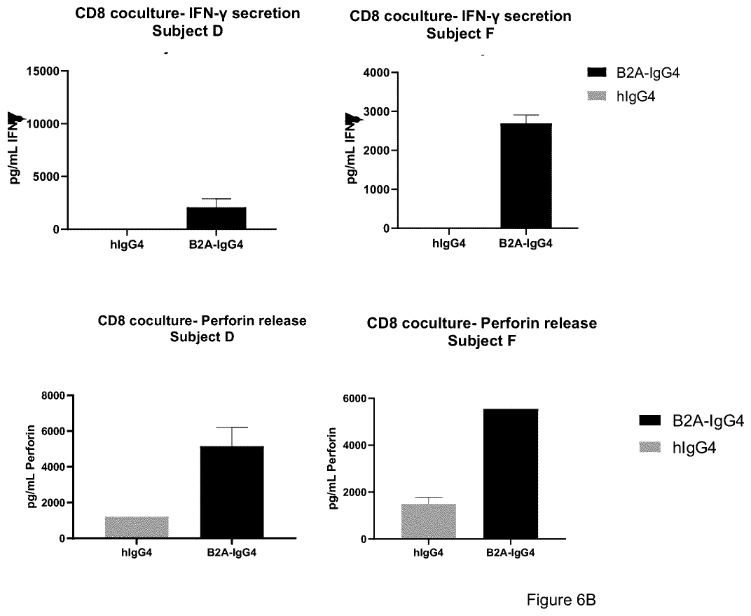

Chimeric antibody clones relieved M2c-macrophage-mediated immune suppression in these M2c/T cell coculture assays, as measured by restoring CD8+ T cell proliferation and IFN-γ secretion and perforin release (FIGS. 6A and 6B, respectively).

### Example 16: LILRB2 Antibodies Rescue IFN-γ Responses of Exhausted T Cells in Coculture with M2c Macrophages

A hallmark of an ineffective anti-cancer immune response is T cell exhaustion in the tumor microenvironment. Exhausted T cells are T cells having decreased cytokine expression and effector function. Reversing T-cell exhaustion and restoring anti-tumor potential represents a promising strategy to treat cancer. The ability of LILRB2 antibodies to rescue functional activity of exhausted T cells from immune suppression by LILRB2 was evaluated by an assay that utilizes exhausted T cells and M2c cells in a coculture to measure the immune suppression mediated by the macrophages.

### T Cell Blast and M2c Coculture Assay

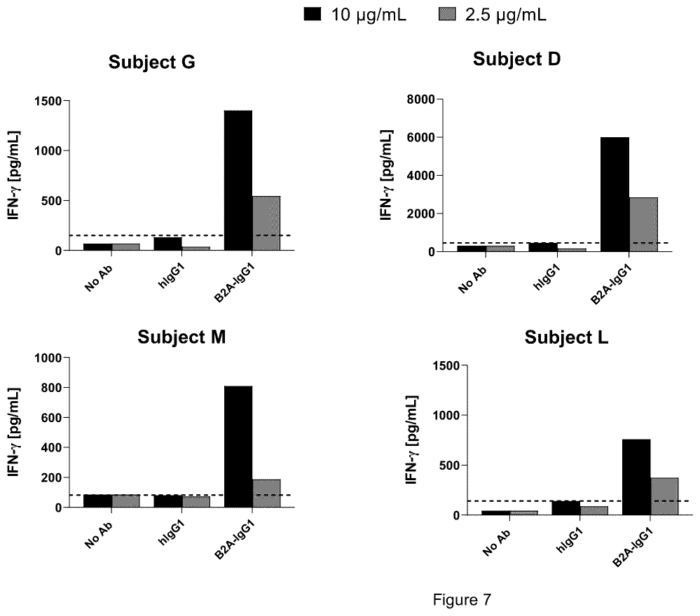

To measure the ability of the LILRB2 antibodies to rescue functional activity of T cell blasts from M2c-mediated immune suppression, culture medium from M2c macrophages was removed and replaced with Assay Medium containing LILRB2 or isotype control antibodies and incubated at 37° C., 5% CO₂ for 2 hours. OKT3 antibody, at a final concentration of 0.25 μg/mL, was added to wells and incubated at 37° C., 5% CO₂ for 30 min. T cell blasts (Example 11) were added last to M2c/LILRB2 antibody mixture+OKT3 at a 1:1 ratio and incubated at 37° C., 5% CO₂ for 72 hours. IFN-γ levels were quantitated from supernatants collected 72 hours after OKT3 stimulation by ELISA (R&D or Meso Scale). B2A-IgG1 rescued the IFN-γ response of exhausted T cells from M2c macrophage mediated immune suppression (FIG. 7). B2A-IgG4 has no activity in this assay (data not shown).

### Example 17: Pharmacokinetic Profiling of Chimeric LILRB2 Antibodies in Humanized FcRn Mice

Therapeutic monoclonal antibodies for cancer treatments are usually of the immunoglobulin G (IgG) subclasses. The half-lives of monoclonal antibodies tested in mice do not correlate with those observed in humans, because human IgG binds mouse neonatal Fc receptor (FcRn) with higher affinity than to human FcRn. FcRn is a major histocompatibility complex (MHC) class I-like heterodimer, containing an Fc binding domain and a β2 microglobulin (β2m). FcRn binds to the Fc portion of an IgG in the acidic environment of the lysosome to prevent its degradation, and FcRn-bound IgG antibodies are recycled back to the extracellular surface where the IgG dissociates from FcRn and goes back into circulation, which in turns allows for increased half-life of these IgG monoclonal antibodies.

Different FcRn transgenic mice were created to knockout the murine FcRn and express human FcRn. Several groups have demonstrated that the PK of human therapeutic antibodies, in these humanized FcRn mice, correlate with human PK and show comparable PK in non-human primates (Petkova S B et al., Int Immunol 2006 18(12):1759-69; Tam S H et al., MAbs 2013 5(3):397-405; Wang W et al., Drug Metab Dispos 2011 39(9):1469-77; Roopenian D C et al., Methods Mol Biol. 2010 602:93-104; Avery L B et al., MAbs 2016 8(6):1064-1078; Proetzel G et al., Methods. 2014 65(1):148-153). For example, the Tg32 and Tg276 mouse strains have been engineered on the C57BL/6 background. These mice are immunocompetent and have all murine immune cells, only the mouse FcRn is deleted and human FcRn is expressed in these models.

Female homozygous Tg32 FcRn mice were purchased at age 6-8 weeks old (Jackson Laboratory, No. 014565) and housed in microisolator cages under specific pathogen-free conditions at the vivarium of Bloodworks Northwest. All procedures were carried out under the institutional guidelines of Bloodworks Northwest's Institutional Animal Care and Use Committee Protocol #5390-01. Mice were identified using ear tags. All mice were acclimated for a minimum of 5 days prior to start of antibody dosing. On the day of dosing, the initial weight was recorded for each mouse. Mice were assigned to two LILRB2 antibody treatment groups: B2A-IgG4 and B2A-IgG1 with 12 mice/group. Twelve mice from each treatment group were split into three blood draw groups with 4 mice per group, and received a single intraperitoneal (IP) dose of 20 mg/kg of B2A-IgG4 or B2A-IgG1 antibodies. Blood was drawn from each group at 3 alternating time points as follows: Group 1: 0.25, 4, 96 hours; Group 2: 1, 24, 168 hours; Group 3: 2, 48, 240 hours.

### Blood Draws and Serum Preparation

Blood was collected via retro-orbital (for 0.25, 1, and 2 hours), submandibular (for 4, 24, and 48 hours), and terminal cardiac puncture (for 96, 168, and 240 hours) bleeds. Whole blood was allowed to clot at RT for a minimum of 30 minutes. Clots were removed by centrifugation (2000×g) for 10 min at 4° C. Serum was aliquoted into 4 new microtubes and frozen at −80° C. until analysis.

### ELISA Assay for Determination of Anti-LILRB2 Antibody Concentration in Mouse Serum

Human LILRB2 capture ELISA was performed to determine antibody concentration for each group. Recombinant human LILRB2-HIS tag proteins were diluted in PBS to 2 μg/mL and added to 384-well high binding ELISA plates (Greiner Bio-One Microlon™) at 25 μL/well and incubated at 4° C. overnight. The plates were washed four times with Wash Buffer, using a microplate washer and then blocked with 90 μL/well of Blocking Buffer E (3% BSA in PBS) for 1 hour at RT. Stocks of B2A-IgG1 and B2A-IgG4 antibodies were diluted in Assay Buffer (0.05% BSA in TBS-T (0.05% Tween® 20 in Tris-buffered saline, pH 7.4)) to prepare a 40 ng/mL top standard concentration. A 15-point, 2-fold serial dilution of standards were diluted in 0.001 to 0.002% mouse serum. Serum samples from anti-LILRB2 treated mice were also diluted at in Assay Buffer. Different dilutions were tested depending on time point of serum collection and dosing antibody. Each standard dilution and mouse serum dilution was assayed in duplicate wells. After blocking, 25 μL/well diluted standards and diluted mouse serum were added to the assay plates and incubated for 1 hour at RT. After primary antibody binding, plates were washed five times with Wash Buffer using a microplate washer. Secondary detection antibody (HRP-goat anti-human IgG Fc specific) was diluted to 1:5000 in assay diluent (1% BSA in PBS) and 25 μL per well was added to plates and incubated at RT in the dark for 1 hour. The plates were washed five times with Wash Buffer, using a microplate washer. After removal of the final wash, 25 μL/well of 1-Step™ Ultra TMB-ELISA Substrate Solution was added and the plates were incubated for 5-10 min at RT, protected from light. After development, the reaction was stopped by addition of 25 μL/well of 0.3 M HCl, and plates were read using an EnVision (Perkin Elmer) microplate reader at 450 nm.

### PK Analysis

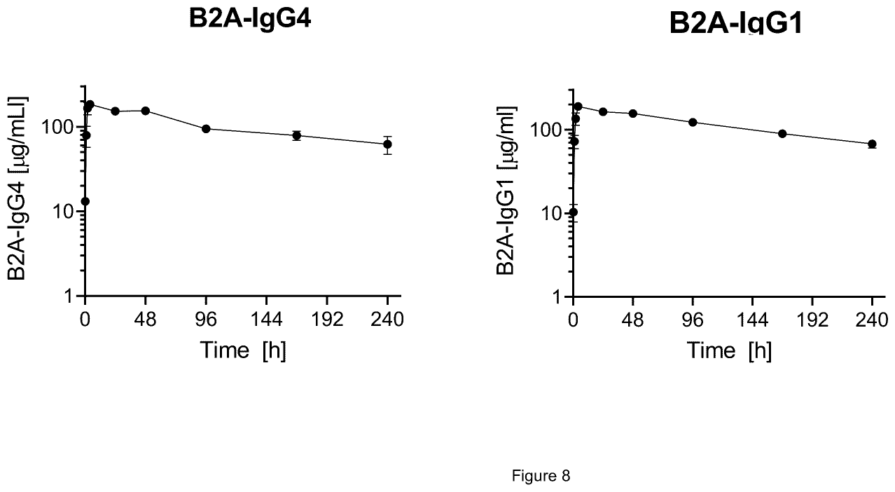

Anti-LILRB2 antibody serum levels were determined from the different blood draws taken at time points 0.25 and up to 240 hours post anti-LILRB2 antibody dosing. Mean B2A-IgG4 and B2A-IgG1 antibody serum levels were generated from 4 mice for each timepoint to generate a composite PK profile. Non-compartmental PK parameters after IP injection were performed with mean serum concentration profiles over time using the Microsoft Excel PK solver 2.0 add-in and PK parameters were calculated using PK solver software (Table 4). Antibody serum exposures were graphed with GraphPad Prism for Windows (GraphPad Software). The B2A-IgG1 and B2A-IgG4 antibodies demonstrated half-lives of 7.0 and 9.9 days in humanized FcRn mice (Table 3 and FIG. 8).

TABLE 3
B2A-IgG4 and B2A-IgG1 have typical
PK profiles in humanized FcRn mice

| Parameters | Unit | B2A-IgG4 | B2A-IgG1 |
|---|---|---|---|
| Dose | mg/kg | 20 | 20 |
| t_{1/2} | H | 238 | 168 |
| t_{1/2} | Days | 9.90 | 7.01 |
| Tₘₐₓ | H | 4 | 4 |
| Cₘₐₓ | μg/mL | 183 | 189 |
| AUC₍₀₋ₜ₎ | μg/mL*h | 24791 | 27797 |
| AUC_{(0-inf)} | μg/mL*h | 46028 | 44238 |

### Example 18: LILRB2 Antibodies Inhibit Tumor Growth in Humanized NSG-SGM3 Mice Bearing Subcutaneous Human SK-MEL-5 Melanoma

Anti-tumor efficacy of B2A-IgG4 was tested in an in vivo humanized tumor model. Female humanized NSG-SGM3 mice were purchased from Jackson Laboratory (JAX West). The immune systems of the triple transgenic NSG-SGM3 mice expressing human IL-3, GM-CSF (CSF2) and SCF (KITLG) (Strain No. 013062) were reconstituted by intravenous injection of human UBC CD34+ hematopoietic stem cells into irradiated 3-week-old NSG-SGM3 mice. Engraftment of human CD45+ cells to assess humanization in peripheral blood was monitored weekly by Jackson Laboratory, and only animals with a minimum engraftment of 25% human CD45+ cells were received from Jackson Laboratory and enrolled in the study.

In Vivo Tumor Xenograft Model and LILRB2 Antibody Dosing:

Female humanized NSG-SGM3 were housed in microisolator cages under specific pathogen-free conditions at the vivarium of Bloodworks Northwest. All procedures were carried out under the institutional guidelines of Bloodworks Northwest's IACUC Protocol #5390-02. Mice were identified using ear tags. All mice were acclimated for a minimum of 5 days prior to study initiation. Mice were inoculated subcutaneously (SC) into the right flank with 2×10⁶ SK-MEL-5 human melanoma cells (HLA class A*02:01; ATCC) in 100 μL PBS with 20% Matrigel (R&D Systems, No. 3632-005-02).

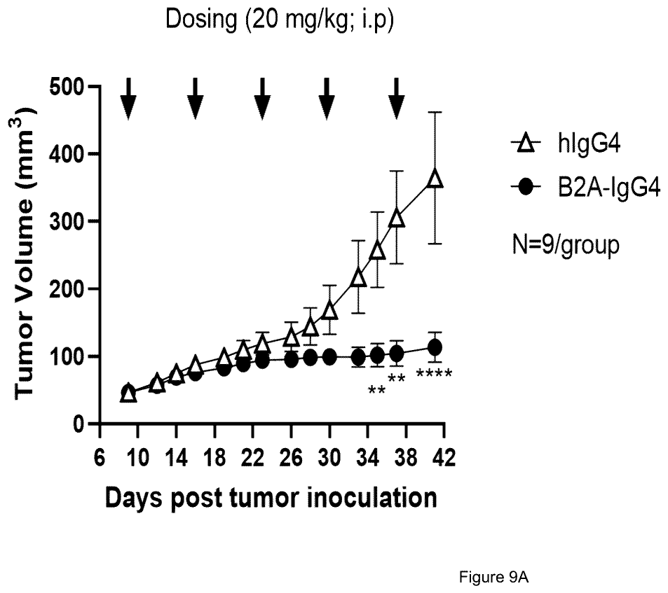

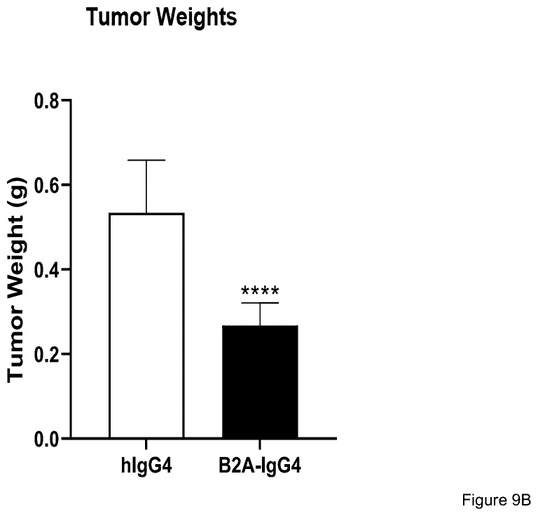

Tumor sizes were measured twice a week using a digital caliper. Tumor volumes were calculated: Tumor volume (mm³)=(L×W²/2) where L is the largest dimension and W is the smallest dimension. When an average tumor size of approximately 50 mm³ was reached, mice were randomized into groups based on both tumor size and engraftment of human CD45+ cells, with each group containing 8-9 mice. Mice were dosed intraperitoneally (IP) with 20 mg/kg B2A-IgG4 or IgG4 isotype control on the day of randomization (Day 9) and again every 7 days (i.e., days 9, 16, 23, 30, and 37 after tumor inoculation) (arrows in FIG. 9a). Mice were sacrificed on day 41, for final tumor weight measurements (FIG. 9b).

Mean tumor growth inhibition (TGI), was calculated using the following formula for all data collection days through Day 27 (Table 5).

$$TGI = [ 1 - \frac{( \overline{X} {Treated}_{( final )} - \overline{X} {Treated}_{( Day 0 )} )}{( \overline{X} {Control}_{( final )} - \overline{X} {Control}_{( Day 0 )} )} ] \times 100 \%$$

Statistical differences in tumor volumes were confirmed using a parametric, RM (Repeated Measure) Two-way ANOVA with Geisser-Greenhouse correction using GraphPad Prism software. Standard errors of the mean were calculated for the tumor volumes daily. The P values were considered significant as follows: *P<0.05; **P<0.01 and ***P<0.001.

Mice treated with anti-LILRB2 B2A-IgG4 achieved 79% tumor growth inhibition (TGI) and 33% tumor regression by day 41 in comparison to IgG4 control-treated group, suggesting the ability of B2A-IgG4 to delay tumor growth (Table 5 and FIG. 9a).

B2A-IgG4 achieved anti-tumor activity in this humanized NSG-SGM3 human SK-MEL-5 melanoma mouse model (FIGS. 9A and 9B and Table 4), while the IgG4 control antibody showed lack of anti-tumor activity in the same tumor model (FIGS. 9A and 9B).

TABLE 4
in vivo tumor growth inhibition (TGI) and tumor
regression of B2A-IgG4 treatment in a SK-MEL-5
tumor model in humanized NSG-SGM3 mice

| | | | | | | Regression |
|---|---|---|---|---|---|---|
| | Tumor Growth Inhibition (%) | | | | | (%) |
| Group | d30 | d33 | d35 | d37 | d41 | d41 |
| B2A-IgG4 | 57 | 69 | 74 | 78 | 79 | 33 |

### Example 19: Antibody Humanization

The rabbit/human chimeric B2A-IgG4 mAb identified was selected for humanization. Humanization was conducted in silico using a proprietary methodology (Fusion Antibodies, Belfast, Northern Ireland). In this system, a model of the parental variable domains is generated to enable structure-guided humanization. Sequences are aligned to a panel of human germline sequences selected for preferential manufacturability properties, and the non-human amino acids are grafted onto the human sequence using a proprietary CDRx™ humanization platform. The first round of humanization conducted for the clone met the EC₅₀ criteria, so only one round of humanization was performed.

Five heavy and five light chains for B2A-IgG4 were generated. The humanized amino acid sequences were submitted to GenScript. GenScript reverse translated and codon optimized each variable region sequence for mammalian cell expression using their proprietary methodology. The genes encoding the signal sequence plus variable region were synthesized at GenScript and cloned into the pTT5 vector in-frame with a human constant IgG4 region for the heavy chain and a human kappa constant region for the light chain. The resulting plasmid vectors, one for the light chain and one for the heavy chain, were transiently co-transfected into HEK293-6E cells (National Research Council Canada (NRC)) and conditioned media were harvested seven days later. The recombinant antibodies were purified by Protein A affinity chromatography. These affinity-purified antibodies were then used to generate binding data.

Success was defined as achieving a binding constant within two-fold of the parental mAb, and in this case the EC₅₀ was determined by ELISA using a rabbit variable region fused to a human IgG4 constant region as parental chimeric mAbs.

Twenty-five IgG4 variants were expressed in mammalian cell culture as a combinatorial library of the five light and five heavy chains. In addition, B2A-IgG4 was included as a transfection control. The conditioned media from these transient transfections were assayed for human IgG concentration. A plate-based ELISA (see Example 2) was performed in which LILRB2-His was immobilized, and binding of humanized mAb variants was detected via an anti-human IgG4 HRP-labeled secondary antibody. The parental rabbit/human chimera clone transfection served as the benchmark EC₅₀ positive control. Antibody titers and EC₅₀ values are shown in Table 5.

TABLE 5
Antibody humanization: Several humanized variants
have better binding EC₅₀ than parental chimera

| Variant | mAb titer [μg/mL] | EC₅₀ [ng/mL] |
|---|---|---|
| B2H4-54 | 231 | 2.25 |
| B2H4-25 | 438 | 4.56 |
| B2H4-24 | 492 | 3.23 |
| B2H4-45 | 511 | 3.61 |
| B2H4-41 | 313 | 3.76 |
| B2H4-51 | 218 | 3.85 |
| B2H4-11 | 476 | 4.20 |
| B2H4-13 | 785 | 4.50 |
| B2H4-25 | 438 | 4.56 |
| B2H4-44 | 348 | 4.60 |
| B2H4-35 | 649 | 4.67 |
| B2H4-43 | 406 | 4.96 |
| B2H4-52 | 431 | 5.37 |
| Parent B2A-IgG4 | 212 | 2.74 |

### Example 20: Binding of Humanized Variant Antibodies to LILRB2 by ELISA and to Cells Expressing LILRB2

Select humanized variant antibodies were purified and tested for their ability to bind human LILRB2 by ELISA, using the method described in Example 2, and binding to HEK293T cells expressing human LILRB2, using the method described in Example 4. Representative data are shown for IgG4 variants in Table 6. The humanized variants of B2A-IgG4 showed similar binding to recombinant and cell-expressed LILRB2 compared to the parental chimera antibody.

TABLE 6
EC₅₀ ELISA LILRB2 binding and binding
to HEK293T-LILRB2 expressing cells

| | ELISA EC₅₀ | EC₅₀ HEK293T- | B_{MAX} HEK293T- |
|---|---|---|---|
| | LILRB2 | LILRB2 | LILRB2 |
| Variant | [ng/mL] | [ng/mL] | [gMFI] |
| B2H4-54 | 2.00 | 0.17 | 10386 |
| B2H4-55 | 2.82 | 0.33 | 10802 |
| B2H4-24 | 2.81 | 0.35 | 10504 |
| B2H4-45 | 2.30 | 0.31 | 10461 |
| B2H4-41 | 3.05 | 0.28 | 10124 |
| B2H4-51 | 3.27 | 0.37 | 10675 |
| B2H4-11 | 3.98 | 0.32 | 9837 |
| B2H4-13 | 3.55 | 0.31 | 9948 |
| B2H4-25 | 2.88 | 0.35 | 10223 |
| B2H4-44 | 2.84 | 0.31 | 10871 |
| B2H4-35 | 2.75 | 0.21 | 9992 |
| Parent B2A-IgG4 | 2.35 | 0.34 | 12057 |

### Example 21: Humanized LILRB2 Antibodies do not Bind to Other LILRB or LILRA Family Members

Select humanized variant antibodies were tested for their ability to bind other LILRB or LILRA family members, using the method and commercially available positive control antibodies of Example 5. The humanized LILRB2 antibody variants did not bind to any members of the LILRA family or to any additional LILRB family members. Representative data are given in Tables 7A and 7B.

### Tables 7A and 7B—Binding of Humanized Variants to LILRB Family Members

| | Antibody Clone | hLILRA1 | hLILRA2 | hLILRA4 | hLILRA5 | hLILRA6 |
|---|---|---|---|---|---|---|
| mAb | [isotype] | Bmax (MFI) | Bmax (MFI) | Bmax (gMFI) | Bmax (MFI) | Bmax (MFI) |
| B2H1-55 | | 783 | 37.7 | 112 | 129 | 214 |
| B2H1-11 | | 776 | 36.1 | 108 | 124 | 210 |
| B2H1-35 | | 766 | 33.6 | 103 | 122 | 202 |
| B2H1-52 | | 855 | 36.1 | 106 | 123 | 219 |
| B2A-IgG1 | | 837 | 40.4 | 104 | 124 | 211 |
| hIgG1 | | 750 | 36.6 | 108 | 121 | 219 |
| Control | | | | | | |
| αLILRA1 | 586326 [mIgG2B] | 6439 | | | | |
| αLILRA2 | 600007 [mIgG1] | | 3554 | | | |
| αLILRA4 | 656688 [mIgG2A] | | | 2419 | | |
| αLILRA5 | 711828 [mIgG2A] | | | | 1638 | |
| αLILRA6 | 921330 [mIgG1] | | | | | 1549 |
| mIgG2A | 20102 | | | 104 | 118 | |
| mIgG2B | 20116 | 319 | | | | |
| mIgG1 | 11711 | | 38.9 | | | 171 |

| | | hLILRB1 | hLILRB2 | hLILRB3 | hLILRB4 | hLILRB5 |
|---|---|---|---|---|---|---|
| | | Bmax | Bmax | Bmax | Bmax | Bmax |
| mAb | Catalog # | (MFI) | (MFI) | (MFI) | (MFI) | (MFI) |
| B2H1-55 | | 41.4 | 10964 | 154 | 40.3 | 138 |
| B2H1-11 | | 39.8 | 11009 | 142 | 38 | 147 |
| B2H1-35 | | 41.3 | 10270 | 146 | 38.5 | 145 |
| B2H1-52 | | 42.3 | 10290 | 159 | 40.5 | 153 |
| B2A-IgG1 | | 41.9 | 11257 | 163 | 41.4 | 147 |
| IgG1 Control | | 41.9 | 49.8 | 156 | 41.5 | 148 |
| αLILRB1 | 292319 [mIgG2B] | 2028 | | | | |
| αLILRB2 | B2Comp2 | | 9632 | | | |
| αLILRB3 | 222821 [mIgG2A] | | | 1368 | | |
| αLILRB4 | 293623 [mIgG2A] | | | | 7698 | |
| αLILRB5 | 395239 [mIgG2B] | | | | | 3501 |
| mIgG2A | 20102 | 43.4 | 125 | 72.7 | 42.1 | |
| mIgG2B | 20116 | | | | | 119 |
| mIgG1 | 11711 | | | | | |

### Example 22: Humanized LILRB2 Variant Antibodies Block LILRB2-Fc Binding to HLA-G

Select humanized IgG4 variant antibodies were tested for their ability to block binding of human LILRB2-Fc to monomer HLA-G by ELISA, using the method described in Example 7. The humanized variants blocked binding of LILRB2-Fc to HLA-G. Representative data are given in Table 9.

TABLE 8
Humanized variants block LILRB2-Fc binding to HLA-G

| | Exp I | Exp II |
|---|---|---|
| | HLA-G IC₅₀ | HLA-G IC₅₀ |
| Variant | [μg/mL] | [μg/mL] |
| B2H4-54 | 4.04 | 4.09 |
| B2H4-55 | 6.89 | 7.17 |
| B2H4-24 | 7.01 | 7.50 |
| B2H4-45 | 6.52 | 7.12 |
| B2H4-41 | 5.78 | 6.76 |
| B2H4-51 | 8.11 | 9.05 |
| B2H4-11 | 7.31 | 7.99 |
| B2H4-13 | 5.85 | 5.97 |
| B2H4-25 | 7.64 | 8.29 |
| B2H4-44 | 7.04 | 7.29 |
| B2H4-35 | 5.16 | 5.14 |

### Example 23: Humanized Variant B2H1-55 Antibody Binds Monocytes and M0 and M2c Macrophages

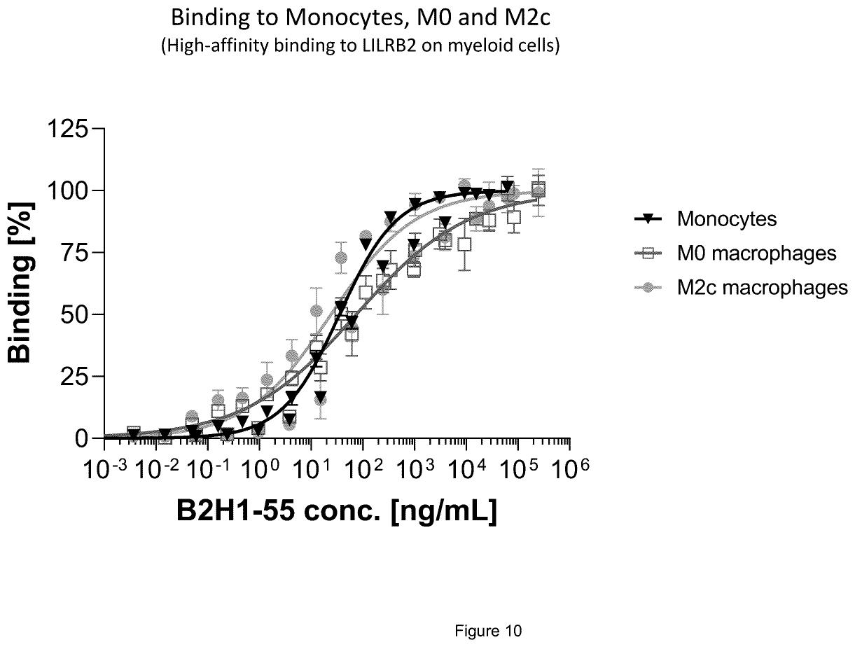

Humanized variant B2H1-55 was tested for binding to monocytes, M0 and M2c macrophages, according to the method described in Example 12. Data compiled from 15 subjects (monocytes), 7 subjects (M0 macrophages), and 10 subjects (M2c macrophages) is shown in FIG. 10, in which a dose titration of AF647-conjugated B2H1-55 is tested. Humanized variant B2H1-55 shows dose-dependent binding to monocytes, M0 and M2c macrophages with an average EC₅₀ of 35.6, 74.8, and 24.3 ng/mL, respectively.

### Example 24: Humanized LILRB2 Antibody B2H1-55 Binds a Panel of Myeloid Cells but not Lymphocytes

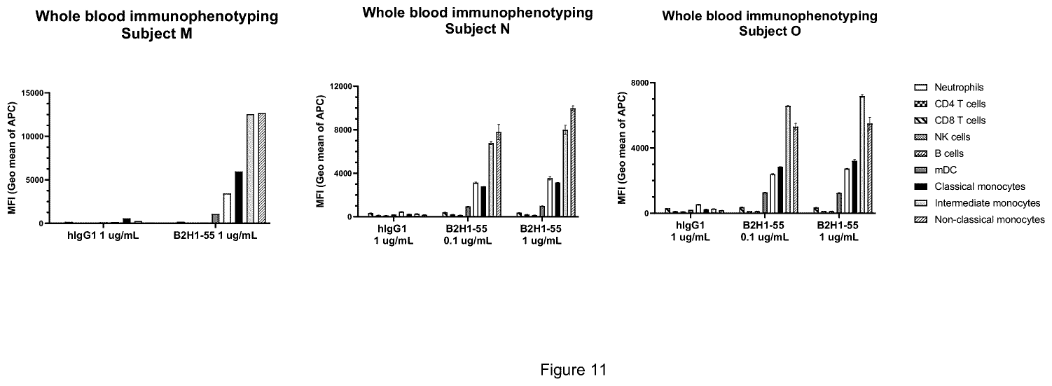

LILRB2 is largely expressed on myeloid cells in whole blood. To determine that the LILRB2 antibodies do not bind to whole blood lymphocytes, and to test the binding of the humanized antibody B2H1-55 to whole blood myeloid cell populations, whole blood immunophenotyping assays were performed. Whole blood from healthy subjects was purchased (Bloodworks Northwest). Tubes containing blood were gently inverted to evenly distribute plasma and cells, then blood was thoroughly mixed with Blocking Mix (10% FBS+500 μg/mL Human IgG1 myeloma plasma (Athens Research)+0.05% NaN₃), pipetted into 96-well deep plates, then incubated for 1 hour at 4° C. Cells were resuspended at the 30-minute mark during incubation with Blocking Mix. Antibody titrations were made in FACS Buffer and added to blood at a final concentration of 10, 1, and 0.1 μg/mL. The mixtures were incubated with primary antibodies for 45 minutes at 4° C., protected from light, and then brought to RT for 15 minutes, for a total incubation time of 1 hour. Red blood cells (RBC) were lysed by three consecutive RBC lysis steps with 1×RBC Lysis Buffer (BD Pharm Lyse™; BD Biosciences, No. 555899) by adding RBC Lysis Buffer to blood mixture, pipetting up and down to fully mix, then incubating for 10 minutes at RT, protected from light. Following incubation, cell plates were sealed, and centrifuged at 200×g for 5 minutes, then supernatant was aspirated from wells. PBS (1×) was added to stop RBC lysis, then centrifuged at 200×g for 5 min, and supernatant removed. Cells were then transferred to 96 well V bottom plates. Viability staining was performed by adding 1:500 dilution of viability dye (Zombie violet, BioLegend, No. 423114) and incubating 20 minutes at RT, protected from light. Cells were resuspended in FACS Buffer and centrifuged to remove viability dye, and supernatant was removed. Cells were then resuspended in FACS Blocking Buffer D and incubated at RT for 15 minutes. Flow cytometry antibody cocktail (fluorophore-conjugated BV421 anti-human CD3 (BioLegend, No. 300434), BV711 anti-human CD4 (BioLegend, No. 300558), APC/Cy7 anti-human CD8 (BioLegend, No. 344714), PE anti-human CD11c (BioLegend, No. 337206), BUV496 anti-human CD14 (BD Biosciences, No. 741200), BUV805 anti-human CD15 (BD Biosciences no. 742057), BV786 anti-human CD16 (BD Biosciences no. 563690), BV605 anti-human CD19 (BioLegend, No. 302244), FITC anti-human CD56 (BioLegend, No. 318304), and PE/Cy7 anti-human HLA-DR (BioLegend, No. 307616) was added directly to wells on top of blocking buffer, then incubated at RT for 30 minutes, protected from light. Following incubation, FACS Buffer was added to wells for washing, then plates were centrifuged at 350×g for 5 minutes and resuspended in 350 μL FACS Buffer for acquisition on FACSymphony™ cytometer. Data from three subjects is shown in FIG. 11, in which B2H1-55 was assayed for binding at two concentrations, compared to hIgG1 isotype control. Humanized LILRB2 antibody B2H1-55 exclusively binds myeloid cells, including classical, non-classical, and intermediate monocytes, myeloid dendritic cells, and neutrophils. B2H1-55 did not interact with human T cells, B cell or NK cells.

### Example 25: Humanized Variant B2H1-55 Bind Neutrophils

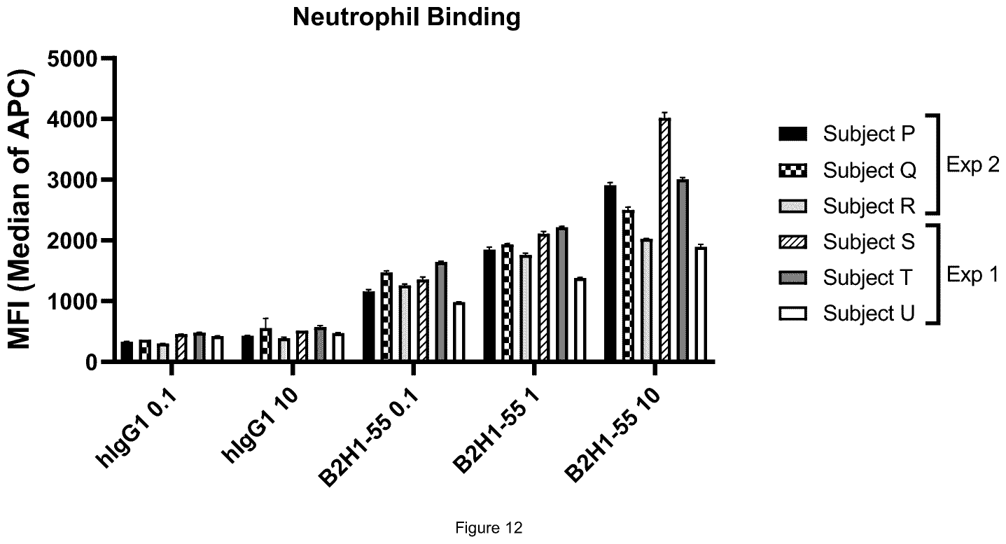

It has been reported that human neutrophils express LILRB2. To determine if the humanized anti-LILRB2 variants bind to and activate human neutrophils, the binding of the humanized antibodies to LILRB2 expressed on neutrophils in whole blood from healthy donors (Bloodworks NW, Seattle, WA) was evaluated. Neutrophils were isolated directly from whole blood by immunomagnetic negative selection, according to manufacturer instructions (StemCell, No. 19666). Following neutrophil isolation, neutrophils were resuspended in Blocking Buffer G (FACS Buffer+10% FBS+2 mM EDTA+0.05% NaN₃+500 μg/mL IgG myeloma plasma), then plated in a 96-well plate at 5×10⁵ cells/mL. Cells were incubated in Blocking Buffer G for 30 minutes at 4° C. Titrations of B2H1-55, conjugated to AF647 in house (Alexa Fluor™ 647 NHS Ester, Thermo Fisher, No. A20106), were made in FACS Buffer at a final concentration of 10, 1 and 0.1 μg/mL. Diluted antibodies were added to neutrophils at a 1:1 dilution, then incubated for 120 minutes at 4° C., protected from light. Following antibody incubation, cells were rinsed with FACS Buffer then centrifuged at 450×g for 5 minutes to pellet cells. Cells were resuspended in viability dye and incubated 10 minutes at RT, protected from light. Following viability staining, cells were rinsed with FACS Buffer, then centrifuged at 450×g for 5 minutes. Cells were resuspended in FACS Buffer and analyzed by flow cytometry using BD FACSymphony™ cytometer. Representative data are shown in FIG. 12, in which a dose-dependent binding of B2H1-55 to neutrophils was observed. Data shown are for six subjects from two independent experiments.

### Example 26: Humanized LILRB2 Antibodies do not Induce Neutrophil Activation

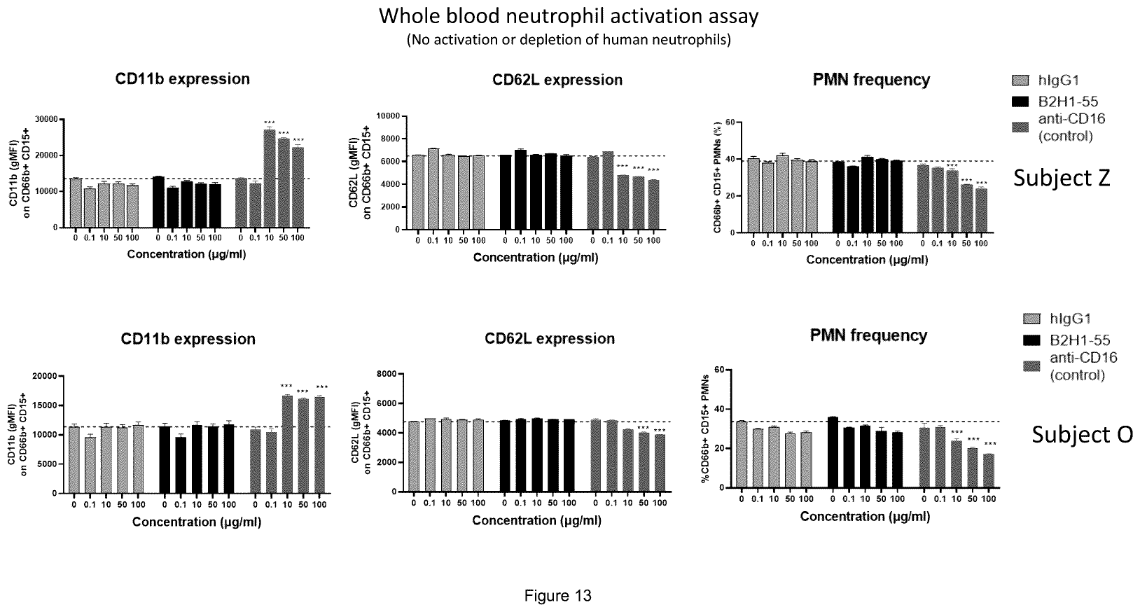

Humanized variant B2H1-55 was tested for its potential to activate human neutrophils in whole blood from healthy subjects. Titration of soluble B2H1-55 humanized variant, an IgG1 isotype control, and a commercial positive control (BioLegend, anti-CD16 clone 3G8) were added to whole blood and incubated for 2 hours at 37° C. in 96-well plates. Final concentration of antibodies in whole blood were 100, 50, 10 and 0.1 pg/ml. Following antibody incubation, cells were rinsed with FACS Buffer then centrifuged at 300×g for 5 minutes to pellet cells and supernatant was discarded. Whole blood cells were then stained with fluorochrome-conjugated BV421 anti-human CD11b (BioLegend, No. 301324), AF647 anti-human CD62L (BioLegend, No. 304818), FITC anti-human CD66b (BioLegend, No. 305104, and BUV395 anti-human CD15 (BD Biosciences, No. 740318) for 20 min at 4° C. Cells were rinsed with FACS Buffer then centrifuged at 300×g for 5 minutes to pellet cells and supernatant discarded. RBC lysis was performed by addition of 200 μL of 1× lysis solution (BD Biosciences, No. 349202, diluted 1:10) to all wells, gentle mixing by pipetting up and down and incubation 3-5 min at RT in the dark. Plates were immediately centrifuged at 200×g for 5 min and supernatant discarded to stop cell lysis. Cells were washed with FACS Buffer at 300×g for 5 min, resuspended in FACS Buffer and assessed by flow cytometry using BD FACSymphony™ cytometer. Changes in neutrophil frequency (CD66b+CD15+) (PMN in FIG. 13) and surface density of activation markers (increase in CD11b and decrease in CD62L) were analyzed with FlowJo software.

Representative data from 2 healthy subjects are shown in FIG. 13, in which the humanized variant B2H1-55 is compared to an anti-CD16 positive control antibody (Clone 3G8), and an isotype control antibody. B2H1-55 did not, upon binding to neutrophils, induce activation of the cells, as indicated by unchanged CD11b expression and retention of surface CD62L. The effects associated with the positive control antibody (FIG. 13) are consistent with reported activity for anti-CD16 antibodies.

### Example 27: Humanized LILRB2 Antibodies Enhance IFN-γ Response in LPS-Stimulated PBMC

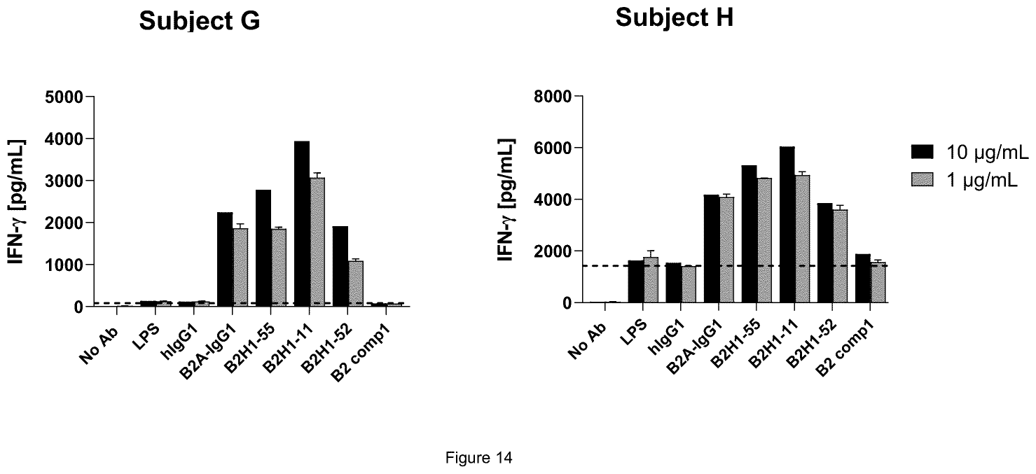

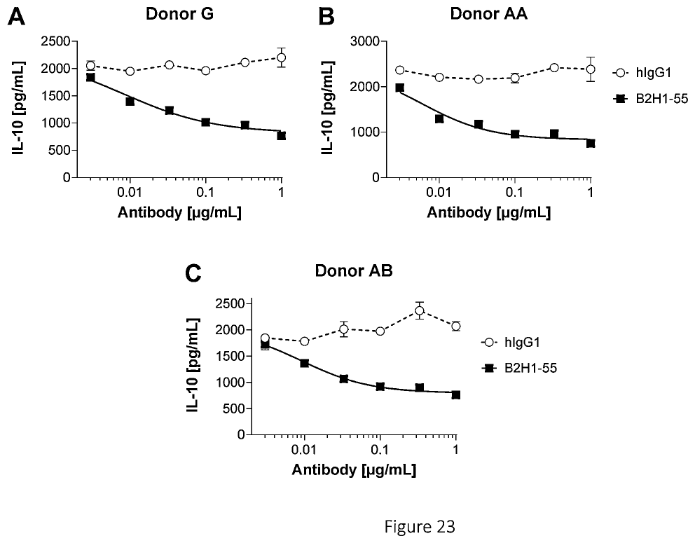

To determine if the humanized variants enhanced the innate immune response of human monocytes by blocking the interaction of LILRB2 with its HLA-ligands, the effect of the variants on IFN-γ production and IL-10 secretion by LPS-stimulated human PBMC was evaluated. Humanized variants were tested for their ability to enhance IFN-γ secretion and IL-10 secretion by LPS-stimulated PBMC, according to the method described in Example 13. Like IFN-γ, IL-10 was quantified in supernatants taken 24 h after LPS stimulation (R&D Systems). Humanized variants induced a pro-inflammatory innate Th1-like phenotype, in which the PBMC displayed enhanced IFN-γ secretion and reduced IL-10 production when stimulated with LPS. Representative IFN-γ data are shown in FIG. 14, in which three humanized variants are compared to the B2A-IgG1 parent, benchmark antibody B2Comp1, and an hIgG1 isotype control antibody. Data shown are for two representative subjects at two antibody concentrations. Humanized LILRB2 variants enhanced the IFN-γ secretion from PBMC stimulated with LPS. As shown in FIG. 23A-C for a representative humanized variant, the humanized anti-LILRB2 variants inhibit LPS-mediated IL-10 release by human PBMC in a dose dependent manner.

Combinations of a humanized anti-LILRB2 variant with a Toll-like receptor 2 (TLR) ligand such as heat-killed Listeria monocytogenes (HKLM, InvivoGen, No: Tlrl-hklm, 2.5×10⁷ cells/mL) and Pam3CSK4 (InvivoGen, No: Tlrl-pms, 100 ng/mL) were also tested. The humanized anti-LILRB2 variants enhanced IFN-γ secretion and reduced IL-10 release by HKLM (2.5×10⁷ cells/mL) or Pam3CSK4 (100 ng/mL) treated PBMCs (data not shown).

### Example 28: Humanized LILRB2 Antibodies Relieve M2c-Mediated Immune Suppression in M2c/T Cell Coculture Assays

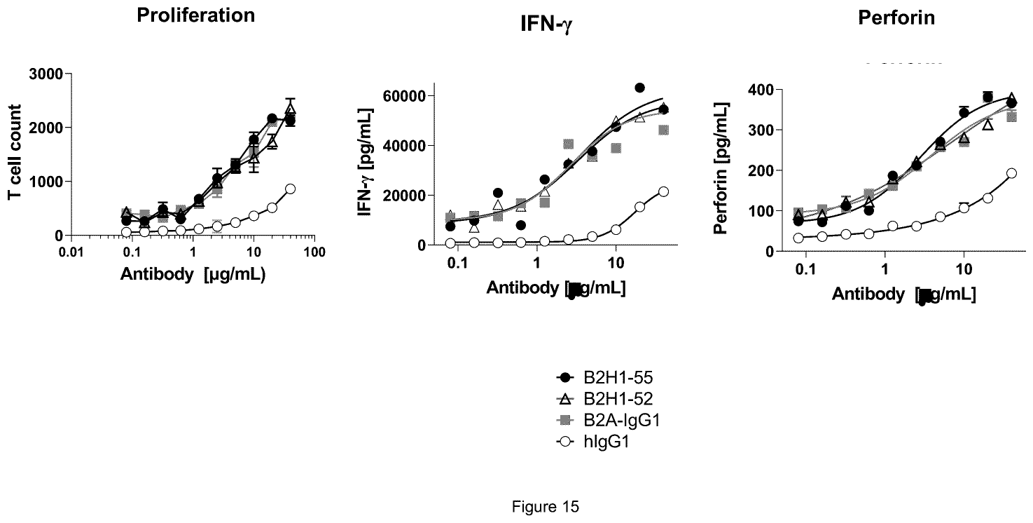

Humanized variants were tested for their ability to restore proliferation, IFN-γ secretion and perforin release by CD8+ T cells cocultured with immunosuppressive M2c macrophages, according to the method described in Example 15. Representative data are shown in FIG. 15. Like the LILRB2 parental chimera B2A-IgG1, humanized LILRB2 variants relieve M2c-mediated immunosuppression by enhancing CD8+ T cell proliferation, IFN-γ secretion and perforin release in a dose-dependent manner.

### Example 29: Humanized LILRB2 Antibodies Prevent Development of Immunosuppressive Macrophages

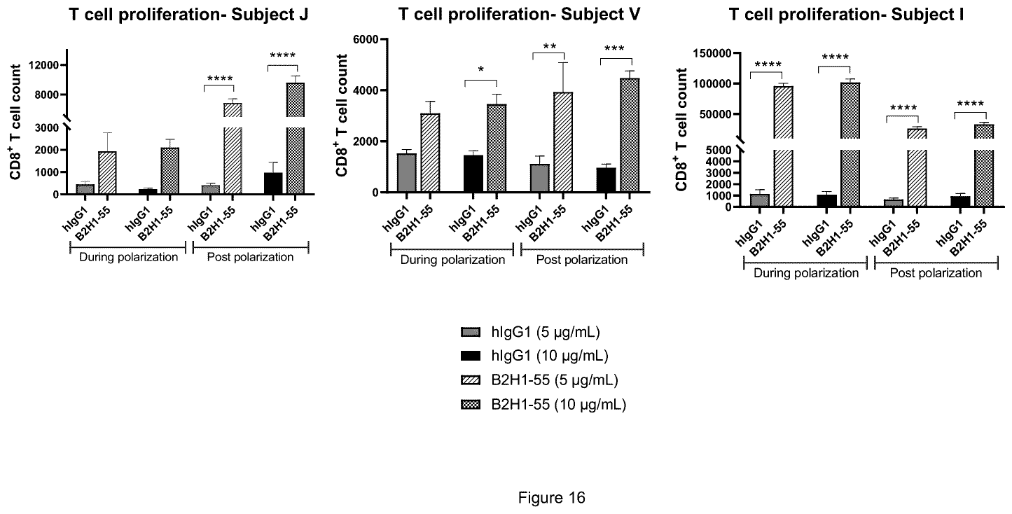

To determine whether the anti-LILRB2 antibodies could interfere with the generation of tumor-associated macrophages, a M2c/CD8+ T cell coculture assay was performed as described in Example 15, with a “pre-regimen” step added during M0 to M2c polarization. For the pre-regimen step, M0 macrophages were polarized to M2c macrophages in the presence of anti-LILRB2 antibodies or isotype control for 2 days (“During polarization”). Following 2-day polarization from M0 to M2c macrophages, antibodies were washed out before 72-hour coculture with CD8+ T cells, and no treatment was added to M2c macrophages following “Post polarization” step. Humanized variant B2H1-55 restored proliferation of CD8+ T cells when added as pre-regimen treatment, implying that the M2c-mediated immunosuppressive effect of these cells has been relieved. Representative data are shown in FIG. 16.

### Example 30: LILRB2 Antibodies Relieve M2c Macrophage-Mediated Immune Suppression in M2c/CD4+ T Cell Coculture

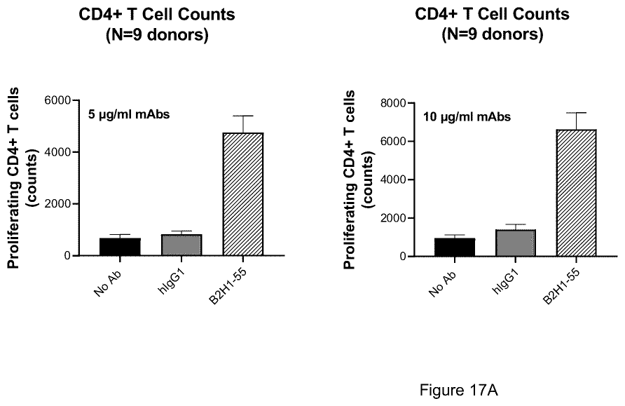

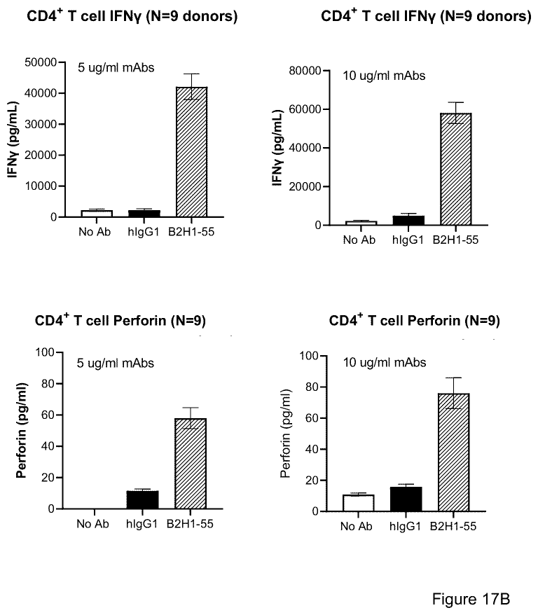

Humanized variant B2H1-55 was tested for its ability to restore proliferative and IFN-γ and perforin secretion by CD4+ T cells cocultured with immunosuppressive M2c macrophages, according to the method described in Example 15. B2H1-55 and IgG1 isotype control was tested at 5 or 10 μg/mL. Representative data are shown in FIGS. 17A and 17B. Humanized variant B2H1-55 relieved M2c-mediated suppression, shown by rescue of CD4+ T cell proliferation (FIG. 17A) and IFN-γ and perforin release (FIG. 17B).

### Example 31: Humanized Variants Rescue IFN-γ Responses by Exhausted T Cells from M2c-Mediated Immune Suppression

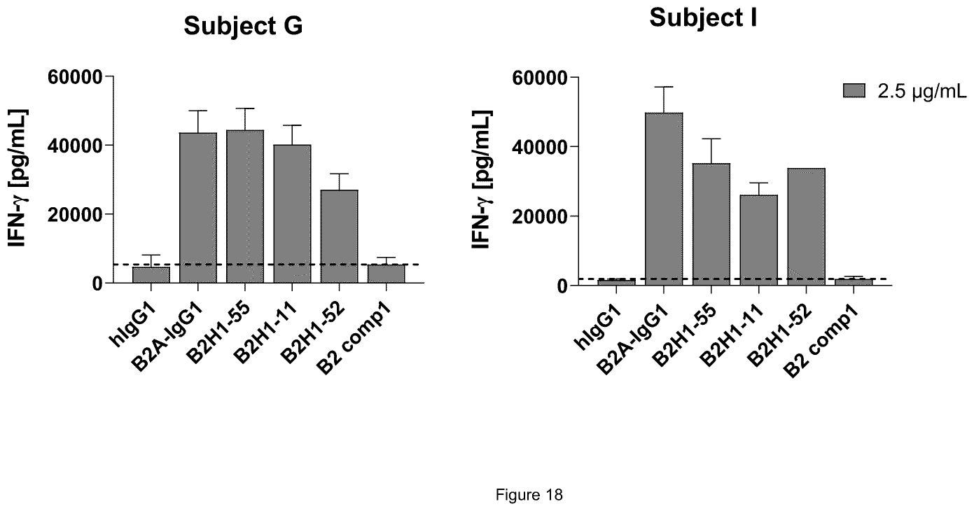

Select humanized variant antibodies were tested for their ability to rescue the IFN-γ responses by exhausted T cells, using the method described in Example 16. Representative data for IFN-γ assays are given in FIG. 18. Like results shown with B2A-IgG1, treatment with humanized variants relieved M2c macrophage-mediated immune suppression as shown by IFN-γ secretion by exhausted T cells.

### Example 32: Humanized LILRB2 Antibodies, in Combination Treatment with PD-1 Antibody, Relieve M2c-Mediated Immune Suppression of Exhausted T Cells

To determine if LILRB2 blockade enhances the efficacy of anti-PD-1 antibodies, humanized variants were evaluated in combination with a PD-1 antibody in the M2c/exhausted T cell coculture assay described above. The experiments followed the methods in Example 16 with the addition of treatment of exhausted T cells with anti-PD-1 or isotype control.

Medium was removed from wells of M2c macrophages and replaced with Assay Medium containing LILRB2 or isotype control antibodies and incubated at 37° C., 5% CO₂ for 2 hours. The final concentrations of anti-LILRB2 antibodies or IgG1 isotype were between 12 and 333 ng/mL. After the initial 2-hour pre-treatment, 50 μL/well of the anti-human CD3 clone OKT3 in Assay Medium (final assay concentration of 0.250 μg/mL) was added and plates were incubated for 30 min at 37° C. before adding the exhausted T cells.

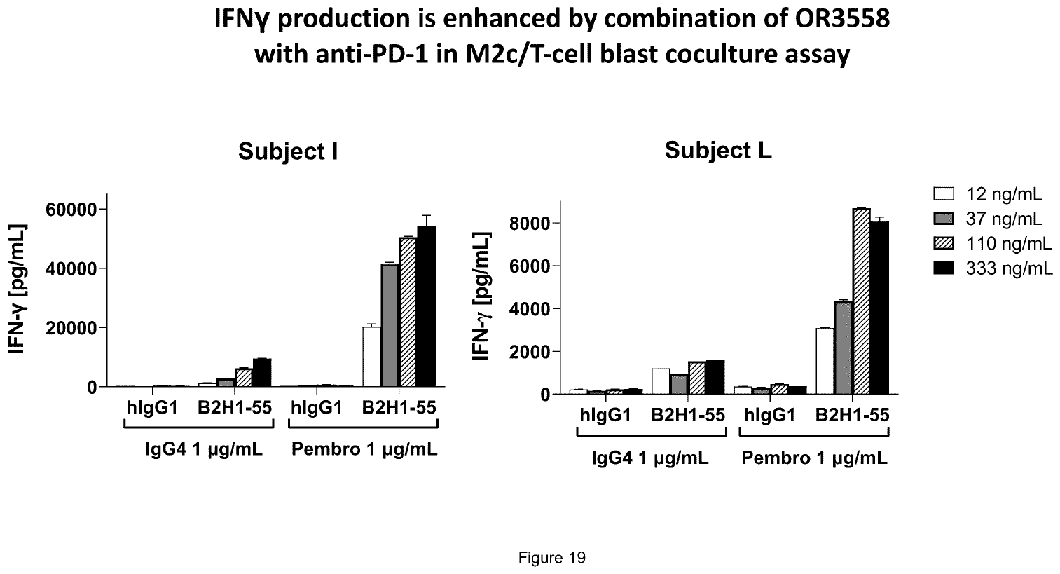

Exhausted T cells were pre-incubated with PD-1 (Pem-hIgG4 S228P) antibody (InvivoGen, No. hpdipe-mab14) or IgG4 isotype control for 2 hours as shown in FIG. 19 The final concentration of anti-PD-1 antibody was 1 μg/mL. Supernatants were collected 72 hours after T cell addition and IFN-γ levels were determined by ELISA (R&D or Meso Scale). Results are shown in FIG. 19. LILRB2-blockade enhanced the efficacy of the anti-PD-1 antibody and rescued the exhausted T cells from M2c-mediated immune suppression, as displayed by enhanced IFN-γ secretion.

### Example 33: Humanized LILRB2 Antibodies Elicit Mild Cytokine Secretion in Whole Blood

Immunomodulatory therapeutic antibodies carry the risk of cytokine release syndrome, a rapid systemic inflammatory response characterized by the secretion of inflammatory cytokines by immune cells. Antibody target binding may induce cytokines by directly activating lymphocytes and myeloid cells, or by interacting with Fcγ receptors on myeloid cells and NK cells. The whole blood in vitro cytokine release assay is a standard assay used to assess the risk of therapeutic antibody-mediated cytokine release syndrome. Whether treatment with humanized LILRB2 variants triggers the release of inflammatory cytokines was evaluated in whole blood from healthy study subjects.

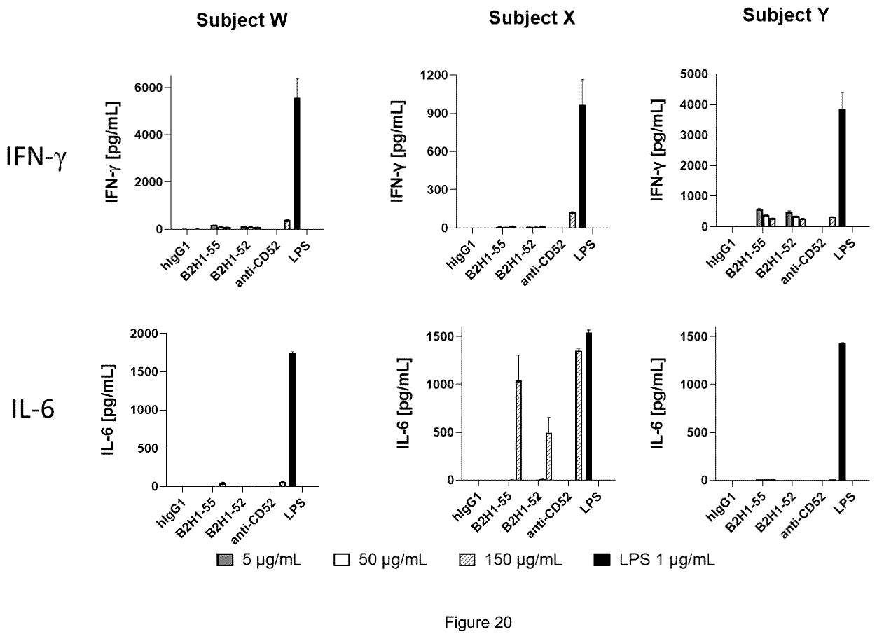

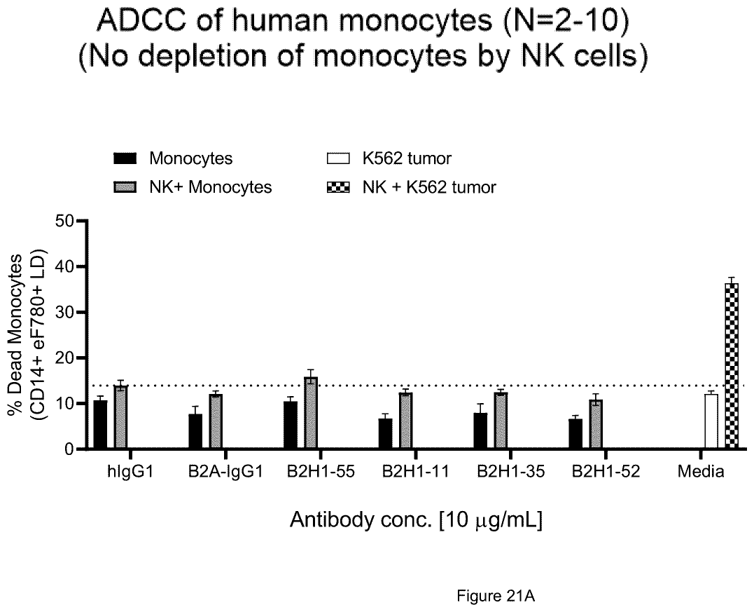

LILRB2 antibodies and control antibodies were diluted to a 10× final concentration in PBS in a dilution plate. Diluted LILRB2 antibodies (25 μL/well) were transferred to 96-well plates. Whole blood from healthy subjects was purchased (Bloodworks NW, Seattle, WA). Whole blood (225 μL/well) was added to LILRB2 antibodies, without mixing, to avoid lysing cells. Blood/antibody mixture was incubated at 37° C., 5% CO₂ for 24-48 hours. Following incubation, plates were centrifuged at 350×g for 5 minutes to pellet cells. Plasma was harvested (65 μL) from the surface of each well, and immediately assessed for IL-6, TNF-α, IFN-γ, and IL-1P cytokine secretion by MSD-ELISA, according to the manufacturer's instructions (Meso Scale Discovery). The release of IL-10, IL-6, IFN-γ, and TNF-α in response to treatment with humanized variants was compared to the cytokine induction by the corresponding human IgG1 isotype, no-treatment control, and an anti-CD52 positive control antibody (alemtuzumab, CAS 216503-57-0, BOC Sciences No. B0084-305393). Representative data from 3 healthy subjects is shown in FIG. 20. In all subjects tested, treatment with LILRB2 humanized variants did not trigger the release of TNF-α or IL-1p (data not shown). In one study subject, minimal IFN-γ secretion was observed following treatment with LILRB2 humanized variants B2H1-55 and B2H1-52 (Subject Y in FIG. 20). In two subjects, minimal IL-6 secretion was observed following treatment at the highest dose of 150 μg/mL with LILRB2 humanized variants, suggesting that these antibodies do not induce cytokine release syndrome in whole blood. Data for one representative subject (Subject X) is shown in FIG. 20, depicting an IL-6 response sometimes observed. Treatment with humanized LILRB2 variants did not trigger the release of IL-6, TNF-α, IFN-γ, or IL-1P cytokine secretion in whole blood of 6 out of 8 healthy subjects. Observed cytokine levels were similar to those induced by the comparator LILRB2 antibody B2Comp1 (data not shown) (IgG4 heavy chain (SEQ ID NO: 43), lambda light chain (SEQ ID NO: 44)), and lower than the those induced by the CD52 antibody positive control.

### Example 34: Humanized Antibody Variants do not Induce ADCC in Human Monocytes or HEK293 Cells Expressing LILRB2

Humanized variants were tested for their ability to induce NK cell-mediated antibody-dependent cellular cytotoxicity (ADCC) against either human monocytes or HEK293T cells expressing human LILRB2. Cytotoxic activity was quantified by the widely used method of quantitation of ADCC by flow cytometry (Yamashita M, 2016 Scientific Reports, 6:19772, DOI: 10.1038/srep19772). Monocytes from frozen PBMCs of healthy subjects were isolated as described in Example 9. NK cells from frozen PBMCs of healthy subjects were isolated using the EasySep™ Human NK cell isolation kit (STEMCELL Technologies, No. 17955) following the manufacturer's instructions.

For the NK cell and human monocyte ADCC assay, isolated monocytes (Target cells) were plated in 96-well low adherence plate in 25 μL/well (104 monocytes/well) in Assay Medium and combined with 25 μL of 4× anti-LILRB2 antibodies or isotype control diluted in culture media for a 10 μg/mL final assay concentration. Target cells and antibodies were incubated for 1 hour at 37° C. Autologous NK cells (Effector cells) were subsequently added (50 μL/well) to the target cells for a final ratio of 8:1 Effector:Target in a final volume of 100 μL/well of Effectors+Targets+antibodies. Cells were incubated for 4 hours at 37° C. and 5% CO₂.

For the NK cells and HEK293T-LILRB2 ADCC assay, HEK293T cells expressing hLILRB2 (Target cells) or mock-transfected cells (negative control target cells) were labeled with CellTrace™ violet dye as described in Example 14. K562 human CML line, which lacks HLA class I and does not express human LILRB2, was included as a positive control target for NK cell killing ability. K562 tumors were labeled with CellTrace™ violet (CTV) similarly to the HEK293T cells. Excess CTV was washed off with pre-warmed Assay Medium and labeled HEK293T-LILRB2 or K562 were resuspended in Assay Medium, then plated in a 96-well low adherence plate in 25 μL/well (10³ Target cells/well) and combined with 25 μL of 4× anti-LILRB2 antibodies or isotype control diluted in culture medium for a 10 μg/mL final assay concentration. Target cells and antibodies were incubated for 1 hour at 37° C. Autologous NK cells (Effector cells) were subsequently added (50 μL/well) to the target cells for a final ratio of 8:1 Effector:Target in a final volume of 100 μL/well of Effectors+Targets+antibodies. Cells were incubated for 4 hours at 37° C. and 5% CO₂.

Detection of NK Cell Killing

Cells were pipetted gently up and down to detach from low adherence plates. Cells were transferred to 96-well v-bottom plates, centrifuged at 300×g for 5 min, supernatant was discarded, and cells were prepared for flow cytometry staining. For the NK and monocyte ADCC assay, cells were resuspended in FACS Buffer containing 2 μL each of human TruStain FcX™ (BioLegend, No. 422302), fluor-labeled anti-human CD16 (BioLegend, No. 302046) and anti-human CD14 (BD Biosciences, No. 563561) antibodies (used to gate monocytes) and incubated at 4° C. for 20 min. Cells were rinsed in FACS Buffer, centrifuged at 300×g for 5 min and supernatant was discarded. Cells were resuspended in 50 μL/well of the e780 viability dye (1:1000 diluted in PBS) and incubated for 15 min at RT. Cells were rinsed and resuspended in FACS Buffer then analyzed by flow cytometry using BD FACSymphony™ cytometer and FlowJO software. NK killing was analyzed by gating and reporting the frequency of CD14+e780-positive cells.

Similar to the NK/monocyte assay, HEK293T or K562 cells were pipetted gently up and down in assay plates, transferred to 96-well v-bottom plates for flow cytometry staining. Cells were resuspended in 50 μL/well of e780 (1:1000 diluted in PBS) and incubated for 15 min at RT. Cells were rinsed in FACS Buffer, resuspended in FACS Buffer, and analyzed by flow cytometry as described. NK killing was analyzed by gating and reporting the frequency of CTV+ e780-positive cells.

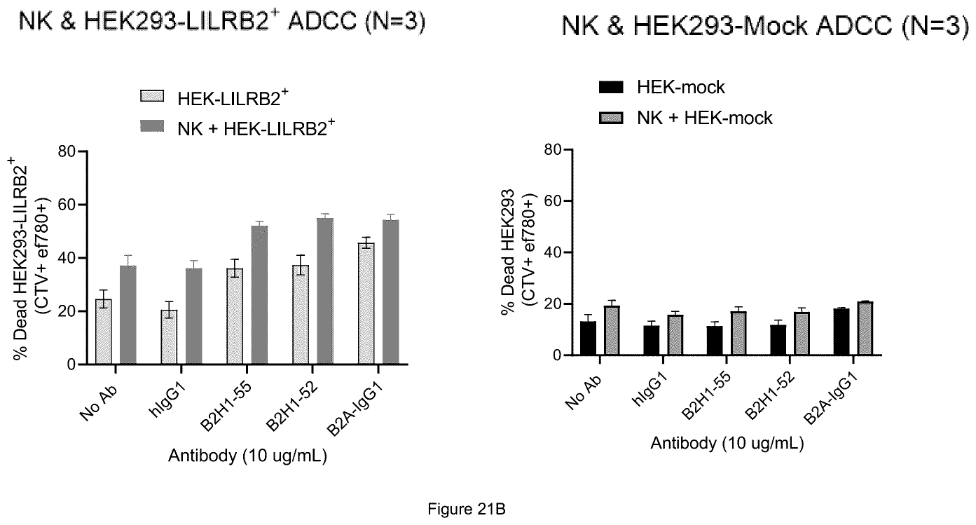

Combined data from 2-10 healthy subjects are shown in FIG. 21A for the NK/monocyte ADCC assay, where hIgG1 isotype control and B2H1-55 antibodies were tested in 10 healthy subjects, and B2H1-11, B2H1-52, B2H1-35, and B2A-IgG1 were tested in two healthy subjects. Three representative subjects are shown in FIG. 21B for the NK/HEK293T-LILRB2 assay. In comparison to the IgG1 isotype control, the parental B2A-IgG1 and humanized variants do not induce ADCC of human monocytes but do induce killing of HEK293T-LILRB2 cells (FIG. 21).

### Example 35: Pharmacokinetic Profiling of Humanized Antibody Variants in Humanized FcRn Mice

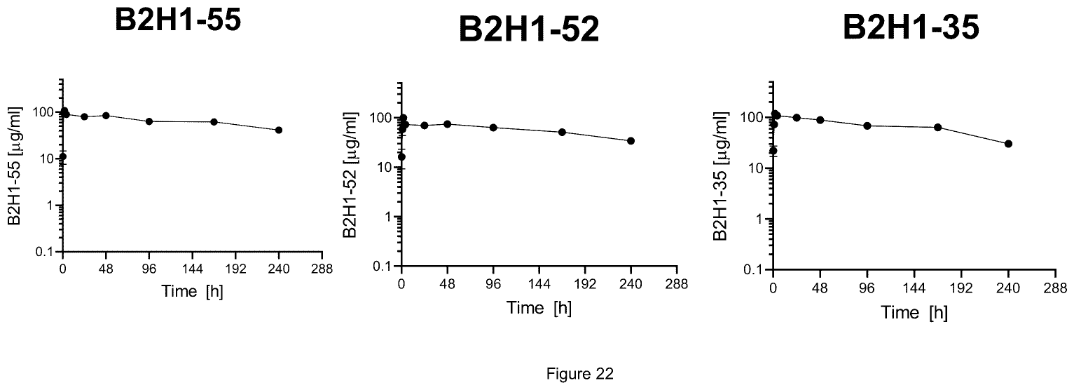

Humanized variants were evaluated for their pharmacokinetic profile in humanized FcRn mice, according to method written in Example 17. Mice were assigned to 3 humanized LILRB2 antibody treatment groups: B2H1-55, B2H1-35, and B2H1-52 with 12 mice/group. Twelve mice from each treatment group were split into three blood draw groups with 4 mice per group and received a single intraperitoneal (IP) dose of 10 mg/kg antibodies. Blood was drawn from each group at 3 alternating time points as follows: Group 1: 0.25, 4, 96 hours; Group 2: 1, 24, 168 hours; Group 3: 2, 48, 240 hours. Serum collection and PK analysis were described in Example 17. PK profile of variants B2H1-55, B2H1-35, and B2H1-52 are shown in Table 9 and FIG. 22. Humanized anti-LILRB2 variants demonstrated a half-life in the range of 5-10 days in humanized FcRn mice, typical for IgG1 antibodies.

TABLE 9
Pharmacokinetic profile of single dose humanized
LILRB2 antibodies in humanized FcRn mice

| Parameters | Unit | B2H1-55 | B2H1-52 | B2H1-35 |
|---|---|---|---|---|
| Dose | mg/kg | 10 | 10 | 10 |
| t_{1/2} | h | 231 | 219 | 139.5 |
| t_{1/2} | days | 9.6 | 9.12 | 5.81 |
| Tₘₐₓ | h | 2 | 2 | 2 |
| Cₘₐₓ | μg/mL | 107.7 | 99.5 | 117.9 |
| AUC₍₀₋ₜ₎ | μg/mL*h | 15664 | 13909 | 16581 |
| AUC_{(0-inf)} | μg/mL*h | 29278 | 24667 | 22671 |

All documents referred to in this application are hereby incorporated by reference in their entirety.

What is claimed is:

1. An antibody product that binds human LILRB2, the antibody product comprising a CDR-H1 set forth in SEQ ID NO: 16, a CDR-H2 set forth in SEQ ID NO: 17, a CDR-H3 set forth in SEQ ID NO: 24, a CDR-L1 set forth in SEQ ID NO: 19, a CDR-L2 set forth in SEQ ID NO: 20, and a CDR-L3 set forth in SEQ ID NO: 21.

2. The antibody product of claim 1, comprising a CDR-H1 set forth in SEQ ID NO: 16, a CDR-H2 set forth in SEQ NO: 17, a CDR-H3 set forth in SEQ ID NO: 18, a CDR-L1 set forth in SEQ ID NO: 19, a CDR-L2 set forth in SEQ ID NO: 20, and a CDR-L3 set forth in SEQ ID NO: 21.

3. The antibody product of claim 1, comprising a CDR-H1 set forth in SEQ ID NO: 22, a CDR-H2 set forth in SEQ NO: 23, a CDR-H3 set forth in SEQ ID NO: 24, a CDR-L1 set forth in SEQ ID NO: 25, a CDR-L2 set forth in SEQ ID NO: 26, and a CDR-L3 set forth in SEQ ID NO: 21.

4. The antibody product of any one of claims 1-3, comprising a heavy chain variable region comprising:
(a) an amino acid sequence at least 80% identical to SEQ ID NO: 1, 6, 7, 8, 9 or 10; or
(b) an amino acid sequence set forth in SEQ ID NO: 1, 6, 7, 8, 9 or 10.

5. The antibody product of any one of claims 1-5, comprising a light chain variable region comprising:
(a) an amino acid sequence at least 80% identical to SEQ ID NO: 2, 11, 12, 13, 14 or 15; or
(b) an amino acid sequence set forth in SEQ ID NO: 2, 11, 12, 13, 14 or 15.

6. The antibody product of any one of claims 1-5, comprising:
(a) a heavy chain variable region comprising SEQ ID NO: 1; and a light chain variable region SEQ ID NO: 2;
(b) a heavy chain variable region SEQ ID NO: 6; and a light chain variable region comprising SEQ ID NO: 11;
(c) a heavy chain variable region comprising SEQ ID NO: 6; and a light chain variable region comprising SEQ ID NO: 12;
(d) a heavy chain variable region comprising SEQ ID NO: 6; and a light chain variable region comprising SEQ ID NO: 13;
(e) a heavy chain variable region comprising SEQ ID NO: 6; and a light chain variable region comprising SEQ ID NO: 14;
(f) a heavy chain variable region comprising SEQ ID NO: 6; and a light chain variable region comprising SEQ ID NO: 15;
(g) a heavy chain variable region comprising SEQ ID NO: 7; and a light chain variable comprising SEQ ID NO: 11;
(h) a heavy chain variable region comprising SEQ ID NO: 7; and a light chain variable region comprising SEQ ID NO: 12;
(i) a heavy chain variable region comprising SEQ ID NO: 7; and a light chain variable region comprising SEQ ID NO: 13;
(j) a heavy chain variable region comprising SEQ ID NO: 7; a light chain variable region comprising SEQ ID NO: 14;
(k) a heavy chain variable region comprising SEQ ID NO: 7; and a light chain variable region comprising SEQ ID NO: 15;
(l) a heavy chain variable region comprising SEQ ID NO: 8; and a light chain variable region comprising SEQ ID NO: 11;
(m) a heavy chain variable region comprising SEQ ID NO: 8; and a light chain variable region comprising SEQ ID NO: 12;
(n) a heavy chain variable region comprising SEQ ID NO: 8; and a light chain variable region comprising SEQ ID NO: 13;
(o) a heavy chain variable region comprising SEQ ID NO: 8; and a light chain variable region comprising SEQ ID NO: 14;
(p) a heavy chain variable region comprising SEQ ID NO: 8; and a light chain variable region comprising SEQ ID NO: 15;
(q) a heavy chain variable region comprising SEQ ID NO: 9; a light chain variable region comprising SEQ ID NO: 11;
(r) a heavy chain variable region comprising SEQ ID NO: 9; and a light chain variable region comprising SEQ ID NO: 12;
(s) a heavy chain variable region comprising SEQ ID NO: 9; and a light chain variable region comprising SEQ ID NO: 13;
(t) a heavy chain variable region comprising SEQ ID NO: 9; and a light chain variable region comprising SEQ ID NO: 14;
(u) a heavy chain variable region comprising SEQ ID NO: 9; and a light chain variable region comprising SEQ ID NO: 15;
(v) a heavy chain variable region comprising SEQ ID NO: 10; and a light chain variable region comprising SEQ ID NO: 11;
(w) a heavy chain variable region comprising SEQ ID NO: 10; and a light chain variable region comprising SEQ ID NO: 12;
(x) a heavy chain variable region comprising SEQ ID NO: 10; and a light chain variable region comprising SEQ ID NO: 13;
(y) a heavy chain variable region comprising SEQ ID NO: 10; and a light chain variable region comprising SEQ ID NO: 14; or
(z) a heavy chain variable region comprising SEQ ID NO: 10; and a light chain variable region comprising SEQ ID NO: 15.

7. The antibody product of any one of claims 1-6, comprising a heavy chain comprising the heavy chain variable domain (VH) and a human heavy chain constant domain (CH).

8. The antibody product of any one of claims 1-7, comprising a light chain comprising the light chain variable domain (VL) and a human light chain constant domain (CL).

9. The antibody product of any one of claims 1-8, comprising a heavy chain comprising the heavy chain variable domain (VH) and a human heavy chain constant domain (CH) and a light chain comprising the light chain variable domain (VL) and a human light chain constant domain (CL).

10. The antibody product of any one of claims 1-9, comprising an IgA, IgD, IgE, IgG, or IgM heavy chain constant domain.

11. The antibody product of claim 7 or claim 9, wherein the heavy chain constant domain is an IgG1 constant domain, an IgG2 constant domain, or an IgG4 constant domain.

12. The antibody product of claim 11, wherein the heavy chain constant domain is an IgG1 constant domain.

13. The antibody product of any one of claims 1-12 comprising a heavy chain amino acid sequence comprising:
(a) an amino acid sequence at least 80% identical to SEQ ID NO: 3, SEQ ID NO: 28, SEQ ID NO: 29, SEQ ID NO: 30, SEQ ID NO: 31, or SEQ ID NO: 32; or
(b) an amino acid sequence set forth in to SEQ ID NO: 3, SEQ ID NO: 28, SEQ ID NO: 29, SEQ ID NO: 30, SEQ ID NO: 31, or SEQ ID NO: 32.

14. The antibody product of any one of claims 1-11, wherein the heavy chain constant domain is an IgG4 constant domain.

15. The antibody product of any one of claims 1-11 and 14, comprising a heavy chain amino acid sequence comprising:
(a) an amino acid sequence at least 80% identical to SEQ ID NO: 4, SEQ ID NO: 33, SEQ ID NO: 34, SEQ ID NO: 35, SEQ ID NO: 36, or SEQ ID NO: 37; or
(b) an amino acid sequence set forth in to SEQ ID NO: 4, SEQ ID NO: 33, SEQ ID NO: 34, SEQ ID NO: 35, SEQ ID NO: 36, or 37.

16. An antibody product that binds human LILRB2, comprising:
(a) an IgG1 heavy chain having an amino acid sequence comprising SEQ ID NO: 1; and a kappa light chain having an amino acid sequence comprising SEQ ID NO: 2;
(b) an IgG1 heavy chain having an amino acid sequence comprising SEQ ID NO: 6; and a kappa light chain having an amino acid sequence comprising SEQ ID NO: 11;
(c) an IgG1 heavy chain having an amino acid sequence comprising SEQ ID NO: 6; and a kappa light chain having an amino acid sequence comprising SEQ ID NO: 12;
(d) an IgG1 heavy chain having an amino acid sequence comprising SEQ ID NO: 6; and a kappa light chain having an amino acid sequence comprising SEQ ID NO: 13;
(e) an IgG1 heavy chain having an amino acid sequence comprising SEQ ID NO: 6; and a kappa light chain having an amino acid sequence comprising SEQ ID NO: 14;
(f) an IgG1 heavy chain having an amino acid sequence comprising SEQ ID NO: 6; and a kappa light chain having an amino acid sequence comprising SEQ ID NO: 15;
(g) an IgG1 heavy chain having an amino acid sequence comprising SEQ ID NO: 7; and a kappa light chain having an amino acid sequence comprising SEQ ID NO: 11;
(h) an IgG1 heavy chain having an amino acid sequence comprising SEQ ID NO: 7; and a kappa light chain having an amino acid sequence comprising SEQ ID NO: 12;
(i) an IgG1 heavy chain having an amino acid sequence comprising SEQ ID NO: 7; and a kappa light chain having an amino acid sequence comprising SEQ ID NO: 13;
(j) an IgG1 heavy chain having an amino acid sequence comprising SEQ ID NO: 7; and a kappa light chain having an amino acid sequence comprising SEQ ID NO: 14;
(k) an IgG1 heavy chain having an amino acid sequence comprising SEQ ID NO: 7; and a kappa light chain having an amino acid sequence comprising SEQ ID NO: 15;
(l) an IgG1 heavy chain having an amino acid sequence comprising SEQ ID NO: 8; and a kappa light chain having an amino acid sequence comprising SEQ ID NO: 11;
(m) an IgG1 heavy chain having an amino acid sequence comprising SEQ ID NO: 8; and a kappa light chain having an amino acid sequence comprising SEQ ID NO: 12;
(n) an IgG1 heavy chain having an amino acid sequence comprising SEQ ID NO: 8; and a kappa light chain having an amino acid sequence comprising SEQ ID NO: 13;
(o) an IgG1 heavy chain having an amino acid sequence comprising SEQ ID NO: 8; and a kappa light chain having an amino acid sequence comprising SEQ ID NO: 14;
(p) an IgG1 heavy chain having an amino acid sequence comprising SEQ ID NO: 8; and a kappa light chain having an amino acid sequence comprising SEQ ID NO: 15;
(q) an IgG1 heavy chain having an amino acid sequence comprising SEQ ID NO: 9; and a kappa light chain having an amino acid sequence comprising SEQ ID NO: 11;
(r) an IgG1 heavy chain having an amino acid sequence comprising SEQ ID NO: 9; and a kappa light chain having an amino acid sequence comprising SEQ ID NO: 12;
(s) an IgG1 heavy chain having an amino acid sequence comprising SEQ ID NO: 9; and a kappa light chain having an amino acid sequence comprising SEQ ID NO: 13;
(t) an IgG1 heavy chain having an amino acid sequence comprising SEQ ID NO: 9; and a kappa light chain having an amino acid sequence comprising SEQ ID NO: 14;
(u) an IgG1 heavy chain having an amino acid sequence comprising SEQ ID NO: 9; and a kappa light chain having an amino acid sequence comprising SEQ ID NO: 15;
(v) an IgG1 heavy chain having an amino acid sequence comprising SEQ ID NO: 10; and a kappa light chain having an amino acid sequence comprising SEQ ID NO: 11;
(w) an IgG1 heavy chain having an amino acid sequence comprising SEQ ID NO: 10; and a kappa light chain having an amino acid sequence comprising SEQ ID NO: 12;
(x) an IgG1 heavy chain having an amino acid sequence comprising SEQ ID NO: 10; and a kappa light chain having an amino acid sequence comprising SEQ ID NO: 13;
(y) an IgG1 heavy chain having an amino acid sequence comprising SEQ ID NO: 10; and a kappa light chain having an amino acid sequence comprising SEQ ID NO: 14; or
(z) an IgG1 heavy chain having an amino acid sequence comprising SEQ ID NO: 10; and a kappa light chain having an amino acid sequence comprising SEQ ID NO: 15.

17. The antibody product of any one of claims 1-9 and 11-16 which is an IgG1 or IgG4 antibody.

18. The antibody product of any one of claims 1-17, comprising a human light chain constant region that comprises a kappa domain or a fragment thereof.

19. The antibody product of any one of claims 1-18, in which the antibody product binds human LILRB2.

20. The antibody product of any one of claims 1-19, that specifically binds human LILRB2 expressed by myeloid cells or cells of a cancer.

21. The antibody product of any one of claims 1-20, in which the antibody product specifically binds to human LILRB2 with a KD from 0.5 nM to 500 nM.

22. The antibody product of any one of claims 1-20, in which the antibody product binds to a human immunosuppressive myeloid cell.

23. The antibody product of claim 21, in which the immunosuppressive myeloid cell is in a tumor microenvironment.

24. The antibody product of claim 21, in which the immunosuppressive myeloid cell is a macrophage, a myeloid dendritic cell, or a myeloid-derived suppressor cell.

25. The antibody product of claim 21, in which the immunosuppressive myeloid cell is an M2a, M2b, M2c, or M2d macrophage.

26. The antibody product of any one claims 1-24, in which the antibody product specifically binds to human M2c macrophages with a K_{D} from 0.5 nM to 500 nM.

27. The antibody product of any one of claims 1-25, in which the antibody product is a monoclonal antibody, a human antibody, a chimeric antibody, a humanized antibody, or a single chain antibody.

28. The antibody product of any one of claims 1-25, in which the antibody product is a monospecific, bispecific, trispecific, or multispecific antibody.

29. The antibody product of any one of claims 1-27, which is bound by an Fc receptor expressed on an immunosuppressive macrophage or other myeloid cell.

30. The antibody product of claim 28, which is bound by CD16 (FcγRIIIa), CD32 (FcγRII), or CD64 (FcγRI) expressed on an immunosuppressive macrophage or other myeloid cell.

31. The antibody product of claim 30, which binds to LILRB2 and to CD16 (FcγRIIIa), CD32 (FcγRII), or CD64 (FcγRI) expressed on the same myeloid cell.

32. The antibody product of claim 30, which binds to LILRB2 on a first cell and to CD16 (FcγRIIIa), CD32 (FcγRII), or CD64 (FcγRI) expressed on a second cell.

33. A method of providing a cancer immunotherapy to a subject in need thereof, in which the cancer is associated with a presence of immunosuppressive macrophages, the method comprising administering to the subject a therapeutically effective amount of an antibody product of any one of claims 1-32.

34. The method of claim 33, in which the antibody product binds to a macrophage and binding of the antibody product to a macrophage results in at least one of the following effects:
(a) promotes activation of a CD4+ T cell, CD8+ T cell, NK cell, or any combination thereof;
(b) promotes proliferation of a CD4+ T cell, CD8+ T cell, NK cell, or any combination thereof;
(c) prevents the polarization of macrophages to immunosuppressive macrophages; and
(d) enhances the innate anti-tumor response.

35. The method of claim 33, in which the activation of a CD4+ T cell, CD8+ T cell, NK cell, or any combination thereof is measured as an enhanced level of IFN-γ, TNF-α, or perforin, or any combination thereof, or a reduced level of IL-10 release.

36. The method of any one of claims 33-35, in which binding of the antibody product to a macrophage is not cytotoxic to the macrophage.

37. The method of any one of claims 33-36, in which binding of the antibody product to a macrophage results in at least one of the following effects:
(a) internalization of the antibody product by the macrophage;
(b) secretion of TNFα, IL-6, perforin, or any combination thereof;
(c) reduced release of IL-10;
(d) activation of a CD4+ T cell, CD8+ T cell, NK cell, or any combination thereof;
(e) proliferation of a CD4+ T cell, CD8+ T cell, NK cell, or any combination thereof; and
(f) promotion of tumor cell killing in a tumor microenvironment.

38. The method of claim 37, in which the binding results in: two or more of (a) through (f); three or more of (a) through (f; four or more of (a) through (f); five or more of (a) through (f); or all of (a) through (f).

39. The method of any one of claims 33-38, in which binding of the antibody product to a macrophage increases an immunostimulatory activity in a tumor microenvironment.

40. The method of any one of claims 33-38, in which binding of the antibody product to a macrophage reduces an immunosuppressive activity of the macrophage.

41. The method of any one of claims 33-38, in which binding of the antibody product to a macrophage reduces a tumor-promoting activity of the macrophage.

42. The method of any one of claims 33-38, in which binding of the antibody product promotes CD4+ T cell activation, CD4+ T cell proliferation, or both CD4+ T cell activation and proliferation.

43. The method of any one of claims 33-38, in which binding of the antibody product promotes CD8+ T cell activation, CD8+ T cell proliferation, or both CD8+ T cell activation and proliferation.

44. The method of any one of claims 33-38, in which binding of the antibody product promotes cytotoxic lymphocyte-mediated killing of cancer cells.

45. The method of any one of claims 33-38, in which binding of the antibody product promotes NK cell-mediated tumor cell killing.

46. The method of any one of claims 33-38, in which binding of the antibody product to a macrophage reduces suppression of cytotoxic T cell-mediated killing of tumor cells in the tumor microenvironment.

47. The method of any one of claims 33-46, in which the cancer is a sarcoma, carcinoma, or blood-borne cancer.

48. The method of claim 47, in which the cancer is glioblastoma multiforme, head and neck cancer, kidney renal clear cell cancer, acute myeloid leukemia, pancreatic adenocarcinoma, skin cutaneous melanoma, stomach adenocarcinoma, testicular germ cell cancer, gastric cancer, Merkel cell carcinoma, dendritic sarcoma, non-small cell lung cancer, papillary thyroid cancer, cutaneous squamous cell carcinoma, or ovarian cancer.

49. The method of claim 47, in which the cells of the cancer overexpress LILRB2.

50. The method of any one of claims 33-49, further comprising administering to the subject an effective amount of an anti-cancer therapeutic.

51. The method of claim 50, in which the anti-cancer therapeutic comprises an immune checkpoint inhibitor.

52. The method of claim 51, in which the immune checkpoint inhibitor is a PD-1 antagonist.

53. The method of claim 52, in which the effective amount of the PD-1 antagonist is an amount effective to relieve immunosuppression of T cells.

54. The method of claim 53, in which the immunosuppression of T cells comprises immunosuppression mediated through interaction of the T cells with myeloid cells expressing PD-L1.

55. A composition, comprising (a) the antibody product of any one of claims 1-32 and (b) an excipient.

56. An article of manufacture, comprising the composition of claim 55 and a container.

57. Use of the antibody product of any one of claims 1-32 or the composition of claim 53, for manufacture of a medicament for treatment of cancer in a subject in need thereof.

58. An isolated nucleic acid, comprising a nucleotide sequence encoding a heavy chain variable region of claim 4.

59. An isolated nucleic acid, comprising a nucleotide sequence encoding a light chain variable region of claim 5.

60. An expression vector comprising the nucleic acid of claim 58.

61. An expression vector comprising the nucleic acid of claim 59.

62. An expression vector comprising the nucleic acids of claims 58 and 59.

63. A host cell comprising the expression vector of any one of claims 60-62.

64. A method of producing a protein comprising an immunoglobulin heavy chain variable domain or an immunoglobulin light chain variable domain, the method comprising:
(a) growing the host cell of claim 63 under conditions such that the host cell expresses the protein comprising the immunoglobulin heavy chain variable region or the immunoglobulin light chain variable region; and
(b) purifying the protein comprising the immunoglobulin heavy chain variable domain or the immunoglobulin light chain variable domain.

65. A method of producing an antibody product that binds human LILRB2, comprising:
(a) growing a host cell comprising the expression vector of claim 62 under conditions such that the host cell expresses a protein comprising the immunoglobulin heavy chain variable region and the immunoglobulin light chain variable region, thereby producing the antibody product; and
(b) purifying the antibody product.

66. A pharmaceutical composition comprising an antibody product of any one of claims 1-32, and a pharmaceutically acceptable excipient.

67. The antibody product of any one of claims 1-32, or the pharmaceutical composition of claim 55, for use in treating a subject having a cancer expressing LILRB2.

68. Use of an antibody product of any one of claims 1-32 in the manufacture of a medicament for treating a cancer expressing LILRB2.

69. A method of detecting LILRB2 in a sample, tissue, or cell using the antibody product according to any one of claims 1-32, comprising contacting the sample, tissue or cell with the antibody product and detecting the antibody product.

70. A method of reducing a biological activity of LILRB2 in a subject in need thereof, said method comprising administering a therapeutically effective amount of the antibody product of any one of claims 1-32 or the pharmaceutical composition of claim 55.

71. The method of claim 70, in which the antibody product mediates depletion of at least one cancer cell expressing LILRB2.

72. The method of claim 70, in which the subject has a tumor having a tumor microenvironment, and anti-tumor immune response within the tumor microenvironment is increased.

73. A method of promoting an immune response in a subject in need thereof, the method comprising administering a therapeutically effective amount of the antibody product of any one of claims 1-32, or the pharmaceutical composition of claim 55.

74. A method of providing a cancer immunotherapy to a subject in need thereof, in which cells of the cancer express LILRB2, the method comprising administering to the subject a therapeutically effective amount of the antibody product of any one of claims 1-32.

75. The method of 74, in which the administering of the antibody product comprises administering an amount the antibody product effective to mediate killing of cells of the cancer through antibody-dependent cellular toxicity.

76. The method of claim 74, in which the administering of the antibody product comprises administering an amount of the antibody product effective to relieve LILRB2-mediated suppression of T cells in the subject.

77. The method of claim 74, further comprising administering to the subject a PD-1 antagonist in an amount sufficient to relieve PD-1/PD-L1 axis-mediated immunosuppression of T cells in the subject.

78. The method of claim 77, in which the PD-1 antagonist is a PD-1 antibody product.

79. The antibody product of any one of claims 1-32, in which the antibody product is detectably labeled or comprises a conjugated toxin, drug, receptor, enzyme, receptor ligand.

80. The antibody product of any one of claim 79, in which the antibody product comprises a therapeutic or cytotoxic agent.

81. A pharmaceutical composition comprising the antibody product of any one of claims 1-32, in which the antibody product reduces or prevents binding of LILRB2 to a ligand thereof and/or reduces or prevents LILRB2-mediated signal transduction and a physiologically acceptable carrier or excipient.

82. The pharmaceutical composition of claim 81, in which the ligand is a human leukocyte antigen A, human leukocyte antigen B, human leukocyte antigen C, human leukocyte antigen G, angiopoietin-like protein 2, angiopoietin-like protein 5, or a combination thereof.

83. The pharmaceutical composition of claim 81 or 82, in which the ligand is expressed on the surface of a myeloid cell or a tumor cell.

84. A method of treating cancer in a subject in need thereof, comprising administering to the subject an effective amount of the pharmaceutical composition of any one of claims 81-83.

85. The method of claim 84, in which the subject has cancer comprising cells expressing or over-expressing a ligand of LILRB2.

86. The method of any one of claims 84-85, in which the antibody product or antigen binding fragment thereof increases an immune response, retards or prevents tumor growth, inhibits tumor-mediated immune suppression, eliminate tumors, depletes or blocks the activity of tumor-associated macrophages so as to alter their activity, decreases tumor-associated macrophage-mediated immune suppression, reduces or reverses T cell suppression, or a combination thereof.

87. The method of any one of claims 84-86, in which the cancer or tumor comprises macrophages expressing LILRB2.

88. The method of any one of claims 84-87, further comprising administering to the subject a second therapeutic agent.

89. The method of claim 88, in which the second therapeutic agent is an immune checkpoint inhibitor.

90. An antibody product that binds human LILRB2, the antibody product comprising a CDR-H1 set forth in SEQ ID NO: 22, a CDR-H2 set forth in SEQ ID NO: 17, a CDR-H3 set forth in SEQ ID NO: 24, a CDR-L1 set forth in SEQ ID NO: 19, a CDR-L2 set forth in SEQ ID NO: 20, and a CDR-L3 set forth in SEQ ID NO: 21.

91. The antibody product of claim 90, the antibody product comprising a CDR-H1 set forth in SEQ ID NO: 16, a CDR-H2 set forth in SEQ ID NO: 23, a CDR-H3 set forth in SEQ ID NO: 18, a CDR-L1 set forth in SEQ ID NO: 25, a CDR-L2 set forth in SEQ ID NO: 26, and a CDR-L3 set forth in SEQ ID NO: 27.


SEQUENCE LISTING (54 sequences)

SEQ ID NO: 1 - protein, 115 aa, synthetic construct
  REGION 1..115: note = synthetic
  REGION 1..115: note = MISC_FEATURE - B2A-VH
QSVEESGGRLVTPGTPLTLTCRVSGFSLSTYAMSWVRQAPGKGLEWIGWIYNDGSTYYAAWAKGRFTFSKTSTTVDLRITSPTPEDTATYFCAREGDYIGYFNIWGQGTLVTVSS

SEQ ID NO: 2 - protein, 111 aa, synthetic construct
  REGION 1..111: note = synthetic
  REGION 1..111: note = MISC_FEATURE - B2A-VL
DVVMTQTPSSVSAALGGTVTIKCQASQSIGSNLAWYQLKPGQRPKLLIYAAATLASGGSSRFKGSGSGTDFTLTISDLECADAATYYCQSYVSGSSDVAFGGGTEVVVKRT

SEQ ID NO: 3 - protein, 445 aa, synthetic construct
  REGION 1..445: note = synthetic
  REGION 1..445: note = MISC_FEATURE - B2A-HC1
QSVEESGGRLVTPGTPLTLTCRVSGFSLSTYAMSWVRQAPGKGLEWIGWIYNDGSTYYAAWAKGRFTFSKTSTTVDLRITSPTPEDTATYFCAREGDYIGYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 442 aa, synthetic construct
  REGION 1..442: note = synthetic
  REGION 1..442: note = MISC_FEATURE - B2A-HC4
QSVEESGGRLVTPGTPLTLTCRVSGFSLSTYAMSWVRQAPGKGLEWIGWIYNDGSTYYAAWAKGRFTFSKTSTTVDLRITSPTPEDTATYFCAREGDYIGYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 216 aa, synthetic construct
  REGION 1..216: note = synthetic
  REGION 1..216: note = MISC_FEATURE - B2A-LCK
DVVMTQTPSSVSAALGGTVTIKCQASQSIGSNLAWYQLKPGQRPKLLIYAAATLASGGSSRFKGSGSGTDFTLTISDLECADAATYYCQSYVSGSSDVAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 118 aa, synthetic construct
  REGION 1..118: note = synthetic
  REGION 1..118: note = MISC_FEATURE - B2H-VH1
QVQLEESGGRVVQPGRSLRLSCAASGFSLSTYAMSWVRQAPGKGLEWIGWIYNDGSTYYAAWAKGRFTISKDTSKTTVFLQMNSLKPDDTATYFCAREGDYIGYFNIWGQGTLVTVSS

SEQ ID NO: 7 - protein, 118 aa, synthetic construct
  REGION 1..118: note = synthetic
  REGION 1..118: note = MISC_FEATURE - B2H-VH2
EVQLVESGGDLATPGGSLRLSCAVSGFSLSTYAMSWVRQAPGKGLEWVGWIYNDGSTYYAAWAKGRFTISKDTSKTTVYLQMTSLRAEDTALYFCAREGDYIGYFNIWGQGTLVTVSS

SEQ ID NO: 8 - protein, 118 aa, synthetic construct
  REGION 1..118: note = synthetic
  REGION 1..118: note = MISC_FEATURE - B2H-VH3
QVQLVESGGRVVQPGTPLRLSCAASGFSLSTYAMSWVRQAPGKGLEWVAWIYNDGSTYYAAWAKGRFTISRDTSKNTLYLQTNSLKPDDTAVYYCAREGDYIGYFNIWGQGTLVTVSS

SEQ ID NO: 9 - protein, 118 aa, synthetic construct
  REGION 1..118: note = synthetic
  REGION 1..118: note = MISC_FEATURE - B2H-VH4
EVQLLESGGRLVQPGTSLRLSCAVSGFSLSTYAMSWVRQAPGKGLEWIGWIYNDGSTYYAAWAKGRFTISKDTSKTTLYLQMNSLTVEDTATYFCAREGDYIGYFNIWGQGTLVTVSS

SEQ ID NO: 10 - protein, 118 aa, synthetic construct
  REGION 1..118: note = synthetic
  REGION 1..118: note = MISC_FEATURE - B2H-VH5
EVQLLESGGGLVQPGTSLTLSCAASGFSLSTYAMSWVRQAPGKGLEWIGWIYNDGSTYYAAWAKGRFTISKDTSKTTLYLQITSLRAEDTATYFCAREGDYIGYFNIWGQGTLVTVSS

SEQ ID NO: 11 - protein, 109 aa, synthetic construct
  REGION 1..109: note = synthetic
  REGION 1..109: note = MISC_FEATURE - B2H-VL1
DIQMTQSPSSLSASVGDRVTITCQASQSIGSNLAWYQLKPGQRPKLLIYAAATLASGVPSRFKGSGSGTDFTLTISSLQSEDAATYYCQSYVSGSSDVAFGGGTEVVVK

SEQ ID NO: 12 - protein, 109 aa, synthetic construct
  REGION 1..109: note = synthetic
  REGION 1..109: note = MISC_FEATURE - B2H-VL2
DVVMTQSPSSVSASLGDRVTITCQASQSIGSNLAWYQQKPGQAPKLLIYAAATLASGVPSRFKGSGSGTDFTLTITSLQPADFATYYCQSYVSGSSDVAFGGGTKVEIK

SEQ ID NO: 13 - protein, 109 aa, synthetic construct
  REGION 1..109: note = synthetic
  REGION 1..109: note = MISC_FEATURE - B2H-VL3
DIEMTQSPSSVSASVGDRVTLTCQASQSIGSNLAWYQQKPGQAPKLLIYAAATLASGVPSRFKGSGSGTDFTLTISSLQPEDFATYYCQSYVSGSSDVAFGGGTEVVVK

SEQ ID NO: 14 - protein, 109 aa, synthetic construct
  REGION 1..109: note = synthetic
  REGION 1..109: note = MISC_FEATURE - B2H-VL4
DIQMTQSPSSVSASVGDRVTITCQASQSIGSNLAWYQQKPGKAPKLLIYAAATLASGVPLRFSGSGSGTDFTLTISNLQPEDSATYYCQSYVSGSSDVAFGGGTKVEIK

SEQ ID NO: 15 - protein, 109 aa, synthetic construct
  REGION 1..109: note = synthetic
  REGION 1..109: note = MISC_FEATURE - B2H-VL5
DVVMTQTPSSVSAALGGTVTIKCQASQSIGSNLAWYQLKPGQRPKLLIYAAATLASGGSSRFKGSGSGTDFTLTISSLEPADAATYYCQSYVSGSSDVAFGGGTEVVVK

SEQ ID NO: 16 - protein, 10 aa, synthetic construct
  REGION 1..10: note = synthetic
  REGION 1..10: note = MISC_FEATURE - CDR-H1 IMGT
GFSLSTYAMS

SEQ ID NO: 17 - protein, 7 aa, synthetic construct
  REGION 1..7: note = synthetic
  REGION 1..7: note = MISC_FEATURE - CDR-H2 IMGT
IYNDGST

SEQ ID NO: 18 - protein, 12 aa, synthetic construct
  REGION 1..12: note = synthetic
  REGION 1..12: note = MISC_FEATURE - CDR-H3 IMGT
AREGDYIGYFNI

SEQ ID NO: 19 - protein, 5 aa, synthetic construct
  REGION 1..5: note = synthetic
  REGION 1..5: note = MISC_FEATURE - CDR-L1 IMGT
SIGSN

SEQ ID NO: 20 - skipped

SEQ ID NO: 21 - protein, 11 aa, synthetic construct
  REGION 1..11: note = synthetic
  REGION 1..11: note = MISC_FEATURE - CDR-L3 IMGT
QSYVSGSSDVA

SEQ ID NO: 22 - protein, 5 aa, synthetic construct
  REGION 1..5: note = synthetic
  REGION 1..5: note = MISC_FEATURE - CDR-H1 Kabat
TYAMS

SEQ ID NO: 23 - protein, 16 aa, synthetic construct
  REGION 1..16: note = synthetic
  REGION 1..16: note = MISC_FEATURE - CDR-H2 Kabat
WIYNDGSTYYAAWAKG

SEQ ID NO: 24 - protein, 10 aa, synthetic construct
  REGION 1..10: note = synthetic
  REGION 1..10: note = MISC_FEATURE - CDR-H3 Kabat
EGDYIGYFNI

SEQ ID NO: 25 - protein, 11 aa, synthetic construct
  REGION 1..11: note = synthetic
  REGION 1..11: note = MISC_FEATURE - CDR-L1 Kabat
QASQSIGSNLA

SEQ ID NO: 26 - protein, 7 aa, synthetic construct
  REGION 1..7: note = synthetic
  REGION 1..7: note = MISC_FEATURE - CDR-L2 Kabat
AAATLAS

SEQ ID NO: 27 - protein, 11 aa, synthetic construct
  REGION 1..11: note = synthetic
  REGION 1..11: note = MISC_FEATURE - CDR-L3 Kabat
QSYVSGSSDVA

SEQ ID NO: 28 - protein, 448 aa, synthetic construct
  REGION 1..448: note = synthetic
  REGION 1..448: note = MISC_FEATURE - B2H-HC1-1
QVQLEESGGRVVQPGRSLRLSCAASGFSLSTYAMSWVRQAPGKGLEWIGWIYNDGSTYYAAWAKGRFTISKDTSKTTVFLQMNSLKPDDTATYFCAREGDYIGYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 448 aa, synthetic construct
  REGION 1..448: note = synthetic
  REGION 1..448: note = MISC_FEATURE - B2H-HC2-1
EVQLVESGGDLATPGGSLRLSCAVSGFSLSTYAMSWVRQAPGKGLEWVGWIYNDGSTYYAAWAKGRFTISKDTSKTTVYLQMTSLRAEDTALYFCAREGDYIGYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 448 aa, synthetic construct
  REGION 1..448: note = synthetic
  REGION 1..448: note = MISC_FEATURE - B2H-HC3-1
QVQLVESGGRVVQPGTPLRLSCAASGFSLSTYAMSWVRQAPGKGLEWVAWIYNDGSTYYAAWAKGRFTISRDTSKNTLYLQTNSLKPDDTAVYYCAREGDYIGYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 448 aa, synthetic construct
  REGION 1..448: note = synthetic
  REGION 1..448: note = MISC_FEATURE - B2H-HC4-1
EVQLLESGGRLVQPGTSLRLSCAVSGFSLSTYAMSWVRQAPGKGLEWIGWIYNDGSTYYAAWAKGRFTISKDTSKTTLYLQMNSLTVEDTATYFCAREGDYIGYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 448 aa, synthetic construct
  REGION 1..448: note = synthetic
  REGION 1..448: note = MISC_FEATURE - B2H-HC5-1
EVQLLESGGGLVQPGTSLTLSCAASGFSLSTYAMSWVRQAPGKGLEWIGWIYNDGSTYYAAWAKGRFTISKDTSKTTLYLQITSLRAEDTATYFCAREGDYIGYFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 445 aa, synthetic construct
  REGION 1..445: note = synthetic
  REGION 1..445: note = MISC_FEATURE - B2H-HC1-4
QVQLEESGGRVVQPGRSLRLSCAASGFSLSTYAMSWVRQAPGKGLEWIGWIYNDGSTYYAAWAKGRFTISKDTSKTTVFLQMNSLKPDDTATYFCAREGDYIGYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 445 aa, synthetic construct
  REGION 1..445: note = synthetic
  REGION 1..445: note = MISC_FEATURE - B2H-HC2-4
EVQLVESGGDLATPGGSLRLSCAVSGFSLSTYAMSWVRQAPGKGLEWVGWIYNDGSTYYAAWAKGRFTISKDTSKTTVYLQMTSLRAEDTALYFCAREGDYIGYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 445 aa, synthetic construct
  REGION 1..445: note = synthetic
  REGION 1..445: note = MISC_FEATURE - B2H-HC3-4
QVQLVESGGRVVQPGTPLRLSCAASGFSLSTYAMSWVRQAPGKGLEWVAWIYNDGSTYYAAWAKGRFTISRDTSKNTLYLQTNSLKPDDTAVYYCAREGDYIGYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 445 aa, synthetic construct
  REGION 1..445: note = synthetic
  REGION 1..445: note = MISC_FEATURE - B2H-HC4-4
EVQLLESGGRLVQPGTSLRLSCAVSGFSLSTYAMSWVRQAPGKGLEWIGWIYNDGSTYYAAWAKGRFTISKDTSKTTLYLQMNSLTVEDTATYFCAREGDYIGYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 445 aa, synthetic construct
  REGION 1..445: note = synthetic
  REGION 1..445: note = MISC_FEATURE - B2H-HC5-4
EVQLLESGGGLVQPGTSLTLSCAASGFSLSTYAMSWVRQAPGKGLEWIGWIYNDGSTYYAAWAKGRFTISKDTSKTTLYLQITSLRAEDTATYFCAREGDYIGYFNIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 216 aa, synthetic construct
  REGION 1..216: note = synthetic
  REGION 1..216: note = MISC_FEATURE - B2H-LC1
DIQMTQSPSSLSASVGDRVTITCQASQSIGSNLAWYQLKPGQRPKLLIYAAATLASGVPSRFKGSGSGTDFTLTISSLQSEDAATYYCQSYVSGSSDVAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 216 aa, synthetic construct
  REGION 1..216: note = synthetic
  REGION 1..216: note = MISC_FEATURE - B2H-LC2
DVVMTQSPSSVSASLGDRVTITCQASQSIGSNLAWYQQKPGQAPKLLIYAAATLASGVPSRFKGSGSGTDFTLTITSLQPADFATYYCQSYVSGSSDVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 216 aa, synthetic construct
  REGION 1..216: note = synthetic
  REGION 1..216: note = MISC_FEATURE - B2H-LC3
DIEMTQSPSSVSASVGDRVTLTCQASQSIGSNLAWYQQKPGQAPKLLIYAAATLASGVPSRFKGSGSGTDFTLTISSLQPEDFATYYCQSYVSGSSDVAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 216 aa, synthetic construct
  REGION 1..216: note = synthetic
  REGION 1..216: note = MISC_FEATURE - B2H-LC4
DIQMTQSPSSVSASVGDRVTITCQASQSIGSNLAWYQQKPGKAPKLLIYAAATLASGVPLRFSGSGSGTDFTLTISNLQPEDSATYYCQSYVSGSSDVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 216 aa, synthetic construct
  REGION 1..216: note = synthetic
  REGION 1..216: note = MISC_FEATURE - B2H-LC5
DVVMTQTPSSVSAALGGTVTIKCQASQSIGSNLAWYQLKPGQRPKLLIYAAATLASGGSSRFKGSGSGTDFTLTISSLEPADAATYYCQSYVSGSSDVAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 448 aa, synthetic construct
  REGION 1..448: note = synthetic
  REGION 1..448: note = MISC_FEATURE - B2Comp1 Heavy Chain IgG4
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 217 aa, synthetic construct
  REGION 1..217: note = synthetic
  REGION 1..217: note = MISC_FEATURE - B2Comp1 Light Chain Lambda
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 45 - protein, 452 aa, synthetic construct
  source 1..452: note = B2Comp2 IgG4 mature
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSSSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 214 aa, synthetic construct
  source 1..214: note = B2Comp2 Kappa mature
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 598 aa, synthetic construct
  REGION 1..440: note = synthetic
  REGION 1..440: note = MISC_FEATURE - hLILRB2 ECD protein mature
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 48 - protein, 446 aa, synthetic construct
  REGION 1..446: note = synthetic
  REGION 1..446: note = MISC_FEATURE - hLILRB2 ECD protein mature
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAYNLSSEWSAPSDPLDILITGQIHGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVHHHHHH

SEQ ID NO: 49 - protein, 455 aa, synthetic construct
  REGION 1..455: note = synthetic
  REGION 1..455: note = MISC_FEATURE - huLILRB2 ECD protein mature
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQADFPMSPVTSAHAGTYRCYGSLNSDPYLLSHPSEPLELVVSGPSMGSSPPPTGPISTPAGPEDQPLTPTGSDPQSGLGRHLGAPPPSGHHHHHHHHHH

SEQ ID NO: 50 - protein, 666 aa, synthetic construct
  REGION 1..666: note = synthetic
  REGION 1..666: note = MISC_FEATURE - huLILRB2 ECD-rIgG1Fc mature
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVRSSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 51 - protein, 670 aa, synthetic construct
  REGION 1..670: note = synthetic
  REGION 1..670: note = MISC_FEATURE - huLILRB2RCD-hIgG1 mature
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 598 aa, synthetic construct; identical to SEQ ID NO: 47
  REGION 1..598: note = synthetic
  REGION 1..598: note = MISC_FEATURE - LILRB+A14:A152 full length protein alternate

SEQ ID NO: 53 - DNA, 1797 bp, synthetic construct
  misc_feature 1..1797: note = synthetic
  misc_feature 1..1797: note = LILRB2full length DNA
  source 1..1797: mol_type = other DNA
atgacccccatcgtcacagtcctgatctgtctcgggctgagtctgggccccaggacccgcgtgcagacagggaccatccccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagatgaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcatggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctgcaggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactag

SEQ ID NO: 54 - protein, 667 aa, synthetic construct
  source 1..667: note = Cyno LILRB2 huIgG1-Fc mature
GILPKPTLWAEPGSVISEGSPVTLRCQGSLQVQEYHLYREKNPASWVRQIRQELVKKGYFAIGFITWEHTGQYRCQYYSHSWWSEPSDPLELVVTGAYSKPTLSALPSPVVASGGNVTLQCDSQVAFDSFTLCKEGEDEHPQRLNCQSHARGWSWAVFSVGPVSPSRRWSYRCYGYISSAPNVWSLPSDLLELLVPGVSKKPSLSVQPGPVVAPGDKLTLQCGSDAGYDRFALYKEGEGDFLQRPVRQPQAGLSQANFLLGPVSRSHGGQYRCSGAHNLSSEWSAPSDPLDILIAGQIRGRPFLSVQPGPKVVSGENVTLLCQSSWQFHAFLLTQAGAADAHLHLRSMYKYPKYQAEFPMSPVTSAHAGTYRCYGSRSSNPYLLSVPSDPLELVVSGPSGGPSSPTTGPTSTCAGPEDQPLTPTGSAPQSGLGRHLGVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK